(19) United States
(12) Patent Application Publication
(10) Pub. No.: US 2021/0059655 A1
(43) Pub. Date: Mar. 4, 2021

(54) MINIMALLY INTRUSIVE CERVICOTHORACIC LAMINOPLASTY SYSTEM

(71) Applicant: Frank H. Boehm, Jr., New Hartford, NY (US)

(72) Inventor: Frank H. Boehm, Jr., New Hartford, NY (US)

(21) Appl. No.: 16/847,026

(22) Filed: Apr. 13, 2020

Related U.S. Application Data
(63) Continuation-in-part of application No. 15/646,615, filed on Jul. 11, 2017, now Pat. No. 10,687,866.
Application No. 15/646,615 is a continuation of application No. PCT/US16/13030, filed on Jan. 12, 2016.
(60) Provisional application No. 62/102,581, filed on Jan. 12, 2015.

(51) Int. Cl. A61B 17/02; A61B 17/70; A61B 17/17

(52) U.S. Cl. CPC A61B 17/025; A61B 2017/0256; A61B 17/1757; A61B 17/7067

(57) ABSTRACT

A special stabilizing anchor is disclosed which is secured to the spinous process, in addition to anchors which are stabilized against the lateral masses. These anchors couple with the spinous process anchor and upon coupling, the connecting stabilizing element is configured such that this element can be actuated, elevating the spinolaminar arch and thus expanding the canal, relieving the stenosis and completing the surgical procedure. A unique aspect of this system is that the lateral mass anchors of different levels can be secured to each other, stabilizing one or more target motion segments. Augmenting this is a system for identifying and extirpating the facet joints and replacing them with graft material to encourage a posterior/facet fusion.

### CROSS-REFERENCE TO RELATED APPLICATIONS

This application claims priority to U.S. Patent Application 62/833,330 (filed Apr. 12, 2019) and is a Continuation-in-Part of U.S. patent application Ser. No. 15/646,615 (filed Jul. 11, 2017) which is a continuation of International Patent Application PCT/US2016/013030 (filed Jan. 12, 2016), which claims priority from U.S. Patent application 62/102,581 (filed Jan. 12, 2015), the entirety of which are incorporated herein by reference.

### BACKGROUND OF THE INVENTION

Cervical degeneration has been one of the most common pathologic processes in human beings for millennia. X-Ray studies of mummified bodies from antiquity show that this was common in adults even then, and today, MRI studies show that more than 80% of asymptomatic volunteers beyond the 5th decade will have significant degeneration.

When this occurs, the spinal column, which is ideally supposed to protect the spinal cord, ultimately becomes a “prison cell on death row,” gradually encasing and compressing the cord until either its vascular supply is interrupted, or the cord is otherwise physically injured by the compression. This results in injury/death to some of the neurons comprising the spinal cord, which ultimately declares itself clinically as a condition referred to as myelopathy—literally “sickness of the spinal cord.” Clinically, this is characterized by a distinct syndrome including evolving weakness, loss of balance, sensory disturbances and reflex disturbances, paradoxically characterized by increased speed/reaction of reflexes, known as “hyperreflexia.” The combination of spastic weakness and hyperreflexia is often referred to as “long tract signs,” referring to the interruption of the corticospinal tracts extending from the motor cortex in the cerebrum to the lower spinal cord. In more recent times, this is broadly referred to in the literature as “Cervical Spondylotic Myelopathy or “CSM.”

One solution to cervical stenosis is to relieve the bony encasement (and ergo the pressure on the spinal cord) in some way, typically referred to in surgical jargon as a “decompressive procedure.” This was initially achieved in the form of a decompressive laminectomy, a procedure in which the surgeon removed part or all of the posterior arch or lamina (see anatomic review below), thus providing the neural elements (spinal cord and nerve roots) with significantly more room, so that the “compression,” is relieved.

Cervical laminectomy was first performed by Walton and Paul to remove a tumor of the cervical spine in 1905. Elsberg performed a laminectomy for a cervical disc in 1925, and multilevel laminectomies for cervical stenosis were the natural evolution of the surgical technique. This was often, but not always successful, and better answers were sought.

One important reason that this procedure would fail is that if the lamina were removed, especially at 3 or more levels, the cervical spine would become relatively unstable. Such patients would eventually become increasingly symptomatic from slippage of the vertebra on one another resulting in sagittal malalignments, ultimately progressing to pathologies such as kyphosis—reversal of the normal curvature of the cervical spine with anterior displacement of the head and upper cervical spine. This will eventually cause further damage to the spinal cord, thus eliminating any salutary effect of the laminectomy to begin with and requiring further surgical intervention such as a fusion.

As surgical techniques evolved, anterior approaches to the cervical spine gained popularity. Introduced by Smith and Robertson but popularized by Cloward in the late 1950's, this approach was initially utilized to treat disc herniations. By the 1980's, extensive anterior resections for CSM were being proposed and became widely utilized within the past two decades.

With the establishment of that procedure, surgical philosophy began to evolve, and it became clear that an anterior versus a posterior approach would be dictated by several factors, including the status of the preoperative sagittal alignment. If the patient was showing a normal “C-shaped curvature,” referred to as “lordosis,” then an anterior or posterior approach could be utilized. If the patient was already kyphotic—showing a reversal of the normal curvature—with the head tilted forward compared to the shoulders, then a posterior approach could exaggerate this condition and an anterior approach should be utilized.

However, stenosis can often extend over 3 levels or more, and in that instance the anterior approach can become challenging. This is particularly a consideration since many of these patients are older and may have other medical issues such that one would like to limit anesthesia time. In such an instance, a posterior approach can be achieved more rapidly. To enhance the posterior approach, Roy-Camille introduced lateral mass screws in the late 1970's, allowing surgeons to immediately stabilize and enhance fusion in the posterior approach. This broadened the use of this approach somewhat, but this was counterbalanced by the technical challenges of placing such screws.

One compromise answer which was proposed by Hirabayshi et al, in 1977 was the so-called open—door laminoplasty. In this posterior approach, troughs were cut into the laminae of a multi-level approach, and the posterior arch is then lifted to one side, decompressing the canal. In this original report, sutures were used to reattach the posterior elements.

It was postulated that this would provide decompression without resulting in significant instability, since the technique included reconstructing the posterior elements. It was thought that this might be particularly useful in the setting of Ossification of the Posterior Longitudinal Ligament (OPLL), which is known to be attended by a high incidence of complications resulting from aggressive decompression. This pathology is known to have an unusually high incidence in Japan, where laminoplasty is an especially popular technique. Others have proposed modifications to this technique. Kurosawa and his colleagues developed a technique referred to a “double door,” laminoplasty in which the spinous processes are split in association with bilateral laminar troughs, with both sides of the posterior arch being rotated posteriorly to open the canal. A prosthesis, either bone graft or biocompatible material, is then secured into the space between the split spinous processes. Multiple studies have shown that these types of procedures will increase the the sagittal diameter of the spinal canal, although they have not been shown to be superior to other surgical techniques in terms of clinical outcomes. An additional modification that has been suggested for use in both techniques is to leave the posterior cervical musculature intact except for the sites of the troughs, referred to as a muscle-sparing approach.

A further modification to the laminoplasty technique was proposed by Ratliff and Cooper, who used small plates to reconstruct the posterior arch after laminoplasty. They thought this offered several advantages, including increasing both the immediate and long-term stability, promoting fusion along the plates, and preventing some of the known complications of laminoplasty, such as anterior displacement of the laminoplasty components with injury of the cord.

Therefore, despite the multiple surgical strategies currently available, precise management of cervical spondylotic myelopathy (CSM) remains controversial. At present, there are 3 techniques utilized: Anterior decompression and fusion, posterior decompression in the form of a multilevel laminectomy, which may include a fusion as well, and laminoplasty techniques. Combinations of these methods have become commonplace, in particular combining an anterior approach with some form of a posterior stabilization and fusion.

Laminoplasty has continued to be embraced by surgeons throughout the world, particularly in Asia. Recently, reports indicate that the screws used in most systems were at risk for failure, screw backout and plate breakage. Those reports notwithstanding, this has continued to be widely used in the surgical community. Many surgeons believe that even with the laminoplasty in place, formal surgical fusion is also necessary. This would routinely dictate the use of lateral mass screw or other posterior cervical fusion system, mandating two different hardware systems.

In distinguishing the present invention from the previous art, it is noted that Cathro, for instance, in U.S. Pat. No. 6,080,157, does not use screws to attach his system but instead utilizes a unilateral “hinged door,” approach, attempting to securely fit an implant into the area between the lamina and lateral mass. Although the art teaches countermeasures to displacement, such a proposal would have the potential of dislodging with relative ease.

To reduce the chances of dislodgement, plating systems have become very popular, including those taught by Angelucci et al. in U.S. Pat. No. 6,635,087; Khanna/U.S. Pat. No. 6,660,007; Taylor/U.S. Pat. No. 7,264,620; Null, et al./U.S. Pat. No. 8,105,366, Mazzuca et al./U.S. Pat. No. 8,147,528; Voellmicke, et al./U.S. Pat. No. 8,133,280 and U.S. Pat. No. 8,470,003; Taylor/U.S. Pat. No. 8,172,875; Konieczynski, et al. in U.S. Pat. No. 8,435,265; Patel/U.S. Pat. No. 8,518,081; Shepard et al./U.S. Pat. No. 8,562,681; Mehdizade/U.S. Pat. No. 8,529,570; Millhouse, et al./U.S. Pat. No. 8,926,664; Chind in U.S. Pat. No. 9,055,982; Robinson in U.S. Pat. No. 9,107,708; Ludwig et al. in U.S. Pat. No. 9,387,014, and finally Mouw in U.S. Pat. No. 9,439,690. Additionally, systems have been proposed for consideration including Squires, et al., in US Pub. 2015/0257789; and Ricica, et al./US Pub. 2015/0265317. Many surgeons now consider such systems the “standard of care.”

All of these systems are secured by screws, which introduce new considerations. These screws are quite small, may not obtain a significant bone purchase, and numerous reports of dislodgement of the screw and even the entire construct are noted. Furthermore, in some of the art cited, the screws are directed into the lateral aspects of the cervical vertebrae in trajectories similar to lateral mass screws; complications including injury to the vertebral artery have also been reported. Furthermore, the plates utilized in these systems are also small, and plate breakage is thought to be a fairly common complication of such systems. Attempting to avoid plate fracture and associated complications, Chung, in U.S. Pat. No. 6,712,852 proposes a cage that must be filled with bone, but still uses screws to become attached to the lamina.

The argument for such technology would be that once a fusion is obtained, the construct would be stabilized. Some surgeons, however, are concerned that the same factors which led to the stenosis in the first place could be at play and ultimately cause “re-stenosis,” and for that reason, resist posterior midline bone graft placement. Williams, in U.S. Pat. No. 7,824,433 teaches the use of a surgical mesh to fully overlie and cover the thecal sac after [multilevel] laminectomy. This would likely be objected to on the same basis.

Disclosed herein is a method for achieving laminoplasty with a unique, useful, novel and nonoperative cervical spine anchor system. This system avoids injuries to the neural elements, vertebral arteries, and other critical structures. One unique feature which distinguishes it from all other current art is that the anchors at different levels can be coupled by securing rods, thus creating a multilevel stabilization which avoids the use of lateral mass screw. In doing so, the system also provides a method by which distraction or compression could be applied to one or more spinal motion segments, further enhancing the utility of this system. Additional features of this system permit the surgeon to position graft material in strategic locations, including extirpating the facet joints and implanting graft material configured to be implanted into such a cavity; purpose-specific graft material can also be positioned in the lateral gutter and utilized to obliterate the osteotomies created in order to achieve the laminoplasty. Such a system would benefit the worldwide spinal surgical community.

### BRIEF DESCRIPTION OF THE INVENTION

The invention relates to the general field of spinal surgery, and specifically to a device for accomplishing cervical/upper thoracic laminoplasty is disclosed. A unique technique for achieving bilateral osteotomies through the lateral aspects of the laminae is disclosed, creating an isolated spinolaminar arch. Furthermore, a special stabilizing anchor is disclosed which is secured to the spinous process, in addition to anchors which are stabilized against the lateral masses. These anchors are then coupled with the spinous process anchor; upon coupling, the connecting stabilizing element is configured such that this element can be actuated, elevating the spinolaminar arch and thus expanding the canal, relieving the stenosis and completing the surgical procedure. A unique aspect of this system is that the lateral mass anchors of different levels can be secured to each other, stabilizing one or more target motion segments. Augmenting this is a system for identifying and extirpating the facet joints and replacing them with graft material to encourage a posterior/facet fusion.

This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter.

### BRIEF DESCRIPTION OF THE DRAWINGS

The drawings are not necessarily to scale; emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings, in which:

FIG. 1 is an anterior view of the osseous cervical spine.

FIG. 2 shows a lateral view of the osseous cervical spine.

FIG. 3 reveals a posterior view of the osseous cervical spine.

FIG. 4 illustrates a transaxial (top) view of a typical cervical vertebra.

FIG. 5A portrays a transaxial view of a stenotic cervical vertebra, demonstrating the critical aspects of this disease process;

FIG. 5B is a sagittal midline hemisection revealing multilevel cervical stenosis.

FIGS. 6A-E demonstrate prior art.

FIG. 7 is an elevational, isolated view of the preferred embodiment of the fully assembled Cervical Minimally Invasive Laminoplasty system [CMIL].

FIG. 8 is a frontal elevational view of the preferred embodiment of the lateral mass anchor and elevating stabilizer.

FIG. 9 demonstrates a lateral elevational view of the isolated preferred embodiment of the spinous anchor.

FIG. 10A-C is a flowchart outlining the implantation procedure.

FIG. 11A demonstrates the isolated incision guide template; 11B shows the sites of the incisions being directed by the guide template.

FIG. 12A is an elevational showing the target spinous process being surgically exposed and stripped of musculotendinous attachments;

FIG. 12B shows the instrument which is used to implant the spinous anchor;

FIG. 12C is the anchor loaded onto the implantation instrument which is being disposed through the incision and into the operative field;

FIG. 12D shows the preferred embodiment of the spinous anchor implanted onto the spinous process.

FIG. 13A shows a surgeon's view of the exposure of the left lateral mass, with a retractor in place; the junction with the lamina is also visualized; 13B shows a transaxial view of the retractor in position.

FIG. 14 displays the analytic algorithm of a transaxial image by the software program which dictates the osteotomy sites.

FIG. 15 demonstrates a transaxial view of a target vertebra showing the proposed sites of the osteotomies.

FIG. 16 illustrates the device which dictates the position of the lateral osteotomies, with a surgeon's view of the device being in position against the lateral and posterior aspects of a right-sided lateral mass.

FIG. 17. Portrays the same view with the lateral guide and drill with the drill cradle having been repositioned into functional position and ready to accept the drill and accomplish the osteotomy.

FIG. 18 is a lateral view the drill as it would be disposed through the cradle.

FIG. 19A/B reveal lateral elevational views of the lateral guide and drill in final position with the drill disposed through and beginning the osteotomy.

FIG. 20 illustrates the completed left sided osteotomy.

FIG. 21 portrays a transaxial view of the medial osteotome.

FIG. 22 is a transaxial view of the instrument as it is utilized against the medial aspect of the lateral mass.

FIGS. 23A/B are lateral and frontal views of the apparatus which implants the lateral anchors.

FIG. 24 displays a transaxial view of the lateral anchors being implanted.

FIGS. 25A/B depict the increase in the total canal cross-sectional area proposed by the software program.

FIG. 26 demonstrates a transaxial view of the CMIL in position prior to augmentation of the spinal canal.

FIG. 27 is a transaxial view of the preferred embodiment of the mechanism for elevating the spinolaminar arch.

FIG. 28 reveals the final configuration of the CMIL secured to an exemplary vertebra with elevation of the central spinolaminar arch.

FIGS. 29A/B illustrate transaxial and lateral elevational views of the prefabricated cadaveric interposition grafts used to promote fusion across the sites of the lateral osteotomies.

FIGS. 30A B show lateral elevational views of a two-level construct of the CMIL before and after positioning the lateral osteotomy grafts.

FIG. 31 demonstrates the preferred embodiment of the method for implanting lateral gutter grafts in a multilevel construct.

FIG. 32A portrays the preferred embodiment of the facet extirpator; FIG. 32B shows the extirpator is use; FIG. 32C shows the facet graft being fitted into place.

FIG. 33 reviews an alternative method for locking the cranial and caudal elements of the spinous anchor to each other.

FIGS. 34A-D also display alternative methods for adjusting the craniocaudal dimension of the spinous anchor, as well as locking the anchor in place;

FIG. 35 reveals an embodiment in which the members of the spinous anchor are monolithic and are coupled in a unique fashion,

FIG. 36A/B display embodiments wherein the members are coupled along the caudal aspect of the spinous process.

FIG. 37 displays a curvilinear plate which is secured to the spinous process with the use of a bone anchor screw.

FIG. 38 illustrates various embodiments of mechanism on the spinous anchor to couple the anchor with the elevating stabilizer.

FIGS. 39A-E portray various alternative embodiments of the elevating stabilizer and its methods for being secured to the anchors.

FIG. 40 illustrates a telescoping configuration of the elevating stabilizer.

FIG. 41 depicts an alternative embodiment of securing the elevating stabilizer to the lateral anchor.

FIG. 42 is an alternative embodiment of the method for locking the lateral anchor in place.

FIG. 43 shows another alternative embodiment for securing the lateral anchor.

FIG. 44 displays a frontal elevational view of an alternative embodiment of the lateral element of the lateral anchor.

FIG. 45 is a frontal view of another method for locking the lateral anchor in place.

FIG. 46 is a lateral view of an alternative device to achieve the lateral osteotomy.

FIG. 47 displays an alternative embodiment of a device to elevate the spinolaminar arch.

FIG. 48 shows an alternative embodiment of the CMIL, in which there is no spinous process anchor but rather anchors secured to the lateral masses as well as to the lateral aspects of the lamina bilaterally.

FIG. 49 shows the coupling of the anchors in the embodiment in FIG. 48

### DESCRIPTION OF THE INVENTION

The invention disclosed herein addresses these and other concerns by providing a device, known hereinafter as the Cervical Minimally Intrusive Laminoplasty (CMIL), as well as a series of implantation devices and methods for use. A principal feature in distinguishing this invention from previous art is that the CMIL does not utilize screws to be secured to the cervical/thoracic vertebrae; hence it is differentiated from much of the previous art. However, in contrast to art the art taught by Cathro, the CMIL is securely anchored to the target vertebrae. Also, in contrast to a number of systems previously proposed, the CMIL elevates a symmetric central spinolaminar arch. Further advantages include a more stable construct and providing the surgeon with the option of incorporating a multilevel construct including a multilevel fusion. As an adjunct to a multilevel fusion, this system offers an integrated fashion by which bone graft substrate can be strategically positioned, promoting multilevel fusion. Yet another advantage of the present disclosure relates to the MIS nature of the procedure, and that as such, musculature and periosteum attached to the spinolaminar arch is largely preserved, and in doing so the blood supply is also preserved. This prevents the elevated bone flap from becoming necrotic sequestrum. No previous identifiable art offers these features.

The preferred embodiment of the CMIL is comprised of one or more anchors secured to the spinous process[es] of the target vertebra[e]. These are then coupled to the engaging ends of connecting stabilizing elements, the trailing/base ends of which are members of the lateral anchors and thereby couple the spinous anchors to lateral anchors, thus completing the construct and stabilizing the laminoplasty. These connecting stabilizing elements are also integral to the preferred mechanism which elevates a central free bony segment, herein referred to as the central spinolaminar arch, created by bilateral osteotomies of the lateral laminae. The arch is comprised of the medial portions of the laminae as well as the central spinous. Elevating this arch achieves the goals of the laminoplasty procedure.

In the preferred embodiment, the components of the CMIL shall be fabricated from surgical grade titanium. Alternatively, some or all of these components can be fabricated from surgical grade stainless steel, or of alloys of any metal, including but not limited to cobalt, nickel, chromium, molybdenum, or of other materials including Nitinol, carbon fiber, polyesters or polyamides, ceramic, PEEK, organic materials such as bone, or any other material known to or proven to be acceptable to the art.

In another aspect of the current invention, a mechanism by which the spinal canal is expanded as the CMIL is deployed is provided. This differs from previous art taught by Farin in U.S. Pat. No. 9,364,335 in that again, no screws are utilized in the CMIL, and the expansion is provided by a different mechanism from that disclosed by Farin.

### Detailed Description of the Drawings

The invention will be best understood if the reader is provided with a fundamental understanding of the pertinent osseous anatomy, and the relationships of various landmarks of the osseous anatomy to key soft tissue structures of the spine. These images are representations of the bony cervical spine, as the object of the invention is to secure to the target vertebra. This, however, recognizes that critical neural and soft tissue structures have not been included in these drawings and that despite their exclusion, these structures must be accounted for within the process of implantation of the invention. Although these soft tissue structures (with the exception of the intervertebral discs) are not illustrated herein, the images will, nevertheless, sufficiently demonstrate the relationships of critical soft tissues such as the spinal cord and nerves to the bony anatomy. When relevant, these structures will be referred to by name in these initial images. The landmarks demonstrated on these images are crucial in implanting the CMIL. It is important to recognize that the cervical spine is the most common site for anatomic anomalies within the spinal column. Such anomalies must be identified and taken into account when surgery using the CMIL is planned. It is imperative to recognize that in certain instances such anomalies, once identified, could represent a relative/absolute contraindication to the use of the CMIL. Also, these images do not include musculotendinous, vascular, or neurologic structures, all of which could be critical in terms of the indications or contraindications of the use of this device.

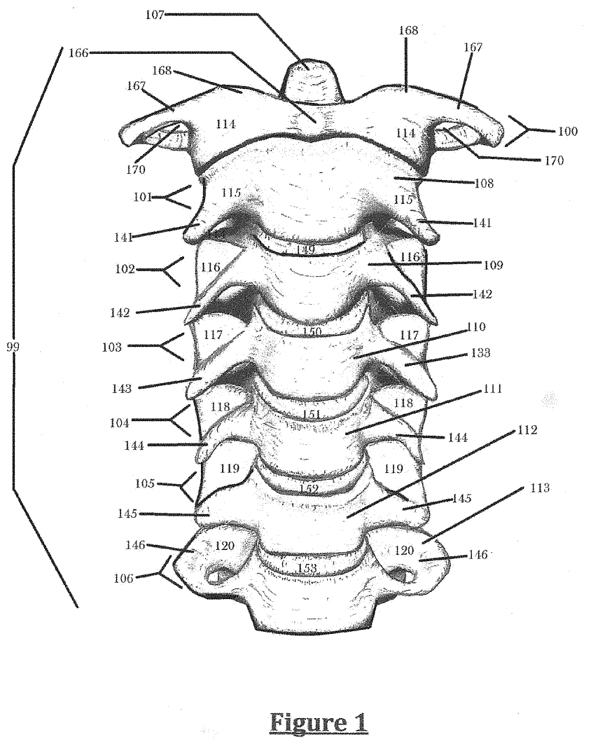

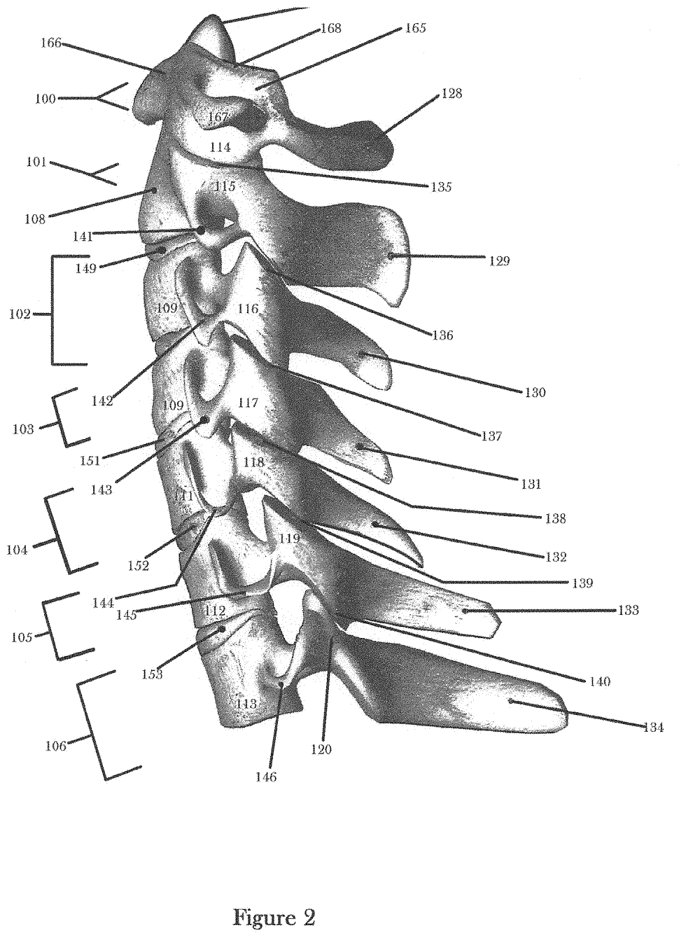

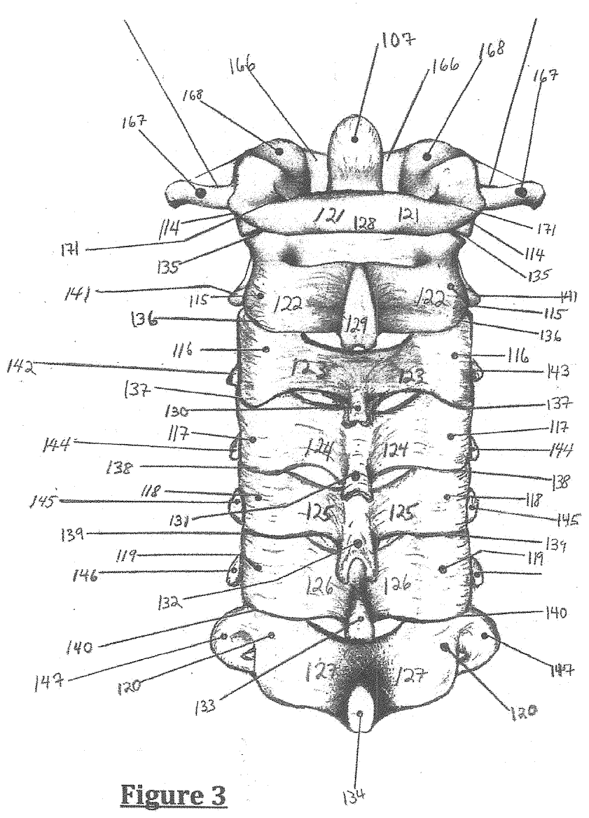

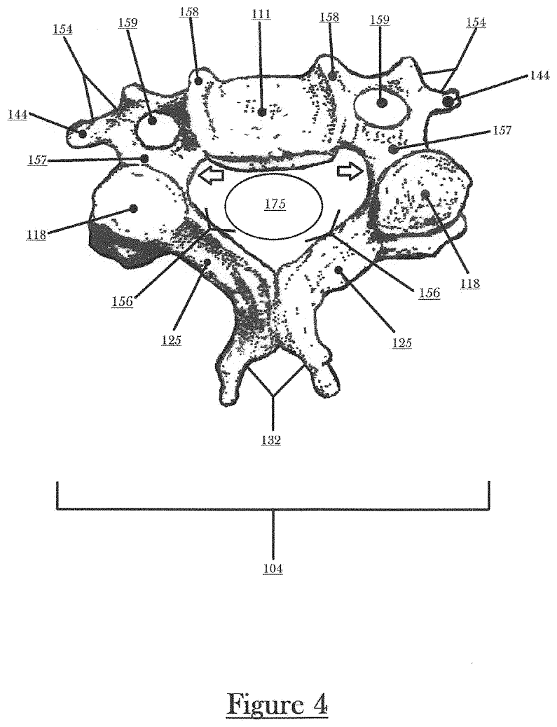

Therefore, turning to the anterior view of the osseous cervical spine 99 in FIG. 1, the seven bones, or vertebrae, named/numbered by convention as C1-C7 can be seen. For the illustrative purposes of this application, these will bear the numbers 100-106 corresponding to C1 100-C7 106. The unique anatomy of the C1-C2 100-101 complex can be, in part, appreciated in this view, and in association with the other views can be more completely understood. It is noted that unlike the other cervical vertebrae, C1 does not have an expanded anterior vertebral body. Moreover, an extension from the C2 vertebra, the Odontoid Process 107 (also known as the Dens), can be seen extending above the top of the anterior arch 166 of C1 100. It will be seen, when other views are scrutinized, that this structure serves as a pivot point around which the C1 vertebra 100 rotates. The mechanism created by this unusual anatomic arrangement is best understood in the lateral and posterior views and is more completely elucidated in FIGS. 2 and 3 below. In addition, one also notes the bilateral transverse processes of C1 167, which provided with the foramina transversaria of C1 170, through which the vertebral artery is transmitted. The course of this important vascular structure is again best seen in FIG. 3. The transverse processes 167 are then continuous with the superior facets 168, which are better seen in the posterior view/FIG. 3 below, and which articulate with the Condyles of the Occipital bone to create the Craniocervical junction (not portrayed). This view demonstrates lateral masses of C1 114 through C7 120, and the bilateral transverse processes of C2 141, C3 142, C4 143, C5 144, C6 145 and C7 146. These transverse processes are provided with grooves which are best seen in the transaxial view in FIG. 4, these grooves configured to transmit the nerve roots exiting the central nervous system and being directed to the upper extremities. Another prominent feature which is best seen in this perspective are the anterior aspects of the intervertebral disc joints, with C2-3 (there is no C1-C2 disc joint) being enumerated as 149 and the C6-C7 space being enumerated as 153. The disc joints are soft tissues structures but are included in these views because of the visual continuity they provide, and because these structures are critically important as these are a major site of degenerative disease.

FIG. 2 is a right lateral view of the skeletonized cervical spine demonstrating additional features of the anatomy. It is to be recalled that this image is only demonstrating the right sided structures in the instances where bilaterality is present. In particular, one notes the curvature of the spine; this illustration shows an idealized curve, referred to as “cervical lordosis,” in which the cervical spine arches anteriorly between C1 and C7 with the point of maximum eccentricity at C4-5. Loss of this normal curvature, or, even worse, reversal of the curvature with the spine bending posteriorly—a condition known as “kyphosis,” are components of pathologies that commonly affect the spine, and surgeons often will attempt to restore this curvature and, in that way, restore the “balance,” of the cervical spine. Also seen well in this view is the expanded anterior vertebral body, which is present in all but the first cervical vertebra C1 100. This vertebra is more poetically known as the Atlas—honoring the protagonist of the Greek Myth who holds up the world; in an analogous fashion, C1 holds up the “globe” of the head. In the embryonic stage, the C1 vertebral body is separated from the remainder of its bony ring and migrates caudally to join the top of C2 101 to become the Odontoid Process 107. The C2 101 is also, and actually more commonly known as the Axis, or the point around which the Atlas, and its “passenger,” the head, pivot; the Odontoid Process 107 principally constitutes that bony pivot point. This provides the healthy individual with the rotational ability of the head on the neck. The vertebral bodies 108-113 can be seen, as well as the spinous process 128-134. It is further noted that the at each level, the facet joints 135-140 represent the posterior articulation of the vertebrae with each other; it should be recognized that the superior facets of C1 168 articulate with the occipital condyles, thus contributing to the occipital-cervical junction, also referred to widely as the craniocervical junction (not illustrated.) It is further understood that the inferior aspect of the C7 106 contributes to the cervicothoracic junction (also not illustrated). Additionally, the transverse processes 141-146 are seen projecting anterolaterally at C2-C7. In discussing FIG. 1 above, it has been mentioned that these transverse processes transmit the cervical nerve roots from the spinal cord, which is found in the central canal created by the anatomical arrangement of the vertebrae.

FIG. 3 allows the reader to achieve a more thorough appreciation of the morphology of the cervical osseous structures as well as their relationship s to critical soft tissue structures. Again, for orientation purposes, the patient's front is at the top of the page, and the bottom is at the bottom. The patient's right side would be located on the viewer's right side; for illustrative purposes, this is a cross-section of the fourth cervical vertebra. The expanded vertebral body 110 is noted to be the anterior stabilizing component. From this component, pedicles 162 extend bilaterally into the lateral masses 117. The posterior arch 163 is comprised of the lateral masses 117 which in reality unite the anterior and posterior elements as the pedicles arise from the facets and are directed anteromedially, while the lateralmost aspect of the lamina 124 is continuous with the lateral mass 117. The spinous process 131 arises from the midline communion of the two laminae 124 (left and right) and is directed posterior. Several important features are best seen in this view: the central canal (labeled) contains the spinal cord (labeled). The nerve roots, (again labeled) arise from the spinal cord and pass through the neural foramina 164, which are canals created at the junction of two vertebrae. The nerve roots then pass along the groove 165 in the transverse process 158 to exit into the soft tissues adjacent to the spine, ultimately contributing to the brachial plexus. A foramen 166 in the transverse process 158 transmits the critical vascular structure, the Vertebral artery (labeled). The positions of the Vertebral Artery and the nerve roots, in particular, subject a patient to significant risk with placement of lateral mass screws. FIG. 4, a transaxial view of a “typical” cervical vertebra (this would be very similar from C3 through the upper thoracic spine), as viewed from the top perspective, allows the reader to further appreciate the relevant bony morphology as well its relationship to critical soft tissue structures.

The C5 vertebra 104 is illustrated as being exemplary in FIG. 4, but the landmarks described are understood to reflect the anatomy of any of the aforementioned vertebrae. Again, for orientation purposes, the patient's front is at the top of the page, and the back is at the bottom. The right side would be located on the viewer's right side. The expanded vertebral body 111 can be recognized conceptually as the anterior stabilizing component. On the lateral aspects of the vertebral body 111 are seen the uncinate processes 158, which participate in the articulation with the vertebra located superiorly to this one. Still further lateral are found the transverse processes 154, which on this view can be noted to be more complex than one might anticipate based on the other views. The neural foramina, as denoted by the bilateral open arrows, are canals which transmit the cervical nerve roots. These arise from the [all important] spinal cord and proceed anterolaterally from the central canal 156 along the groove in the transverse processes 154 to exit into the soft tissues adjacent to the spine, ultimately contributing to the brachial plexus. Also noted is the Foramen Transversarium 159, which transmits the Vertebral artery. The positions of the Vertebral Artery and the nerve roots, in particular, subject a patient to significant risk with placement of lateral mass screws. The pedicle 157, which is in reality a component of the transverse process complex, serves to connect the anterior structures to the posterior arch, thus creating the central canal 156. The posterior arch is comprised of the lateral masses 118 which create the superior and inferior facet joints for the vertebra being examined. Additionally, the laminae 125 and the midline spinous process 132 complete the posterior arch. The central canal 156 contains the spinal cord 175 (not shown in this view), responsible for the transmission of all information to and from the brain, and whose integrity is mandatory for locomotion and many essential functions, is the most critical structure which must be considered with the implantation of any surgical instrumentation into this area of the spine. Additionally, injuries to the vertebral artery or nerve roots must also be avoided as they carry with them substantial deleterious consequences. This view is particularly helpful in showing that the laminae 125 arise from the base of the spinous process 132, and initially assume a rather steep angle in the A-P plane as they move laterally. They then become more horizontal as the continue laterally to become the lateral masses 118. The ideal place for implantation of the laminar anchor is at the transition point, as this is lateral from the spinal cord 175. Such positioning is slightly medial to the lateral masses, where shingling would exclude the possibility of implantation. This ideal position is just slightly less than halfway between the midline and the lateral most edge of the spine, as seen in the posterior view.

This review isn't merely an academic exercise. Rather, it permits one to more completely understand the main and other objectives of the invention and view the invention and its objectives in the proper perspective. Having completed the review, the anatomic terms defined therein will be utilized for the balance of the disclosure.

### Detailed Description of the Drawings Demonstrating the Relevant Pathologic Anatomy

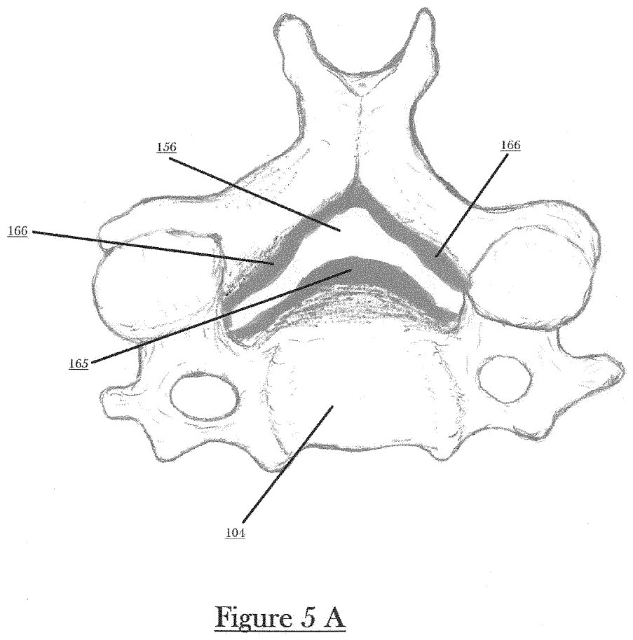

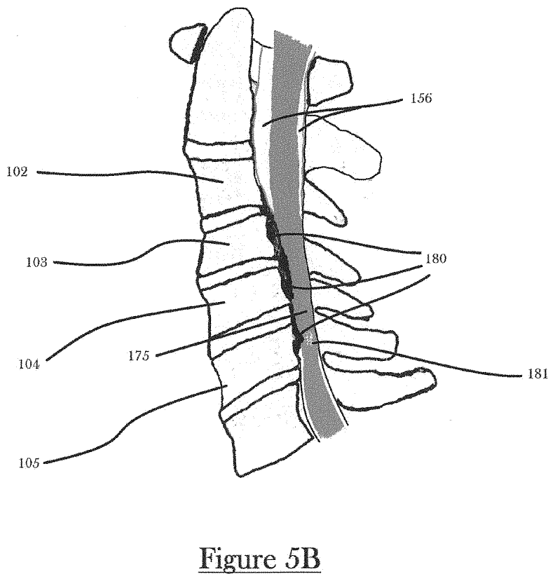

The critical features of the pathologic findings in cervical stenosis can be seen in FIG. 5 A, a transaxial image of C5 104, being used as an exemplary image representative of any vertebral level afflicted with this pathology. Here it can be seen that the spinal canal 156 has been greatly reduced by a large, posterior calcified disc/osteophyte 165. Also noted are bilateral posterior narrowing 166 related to calcification of the yellow ligament. Other pathologies which can contribute to such a condition include ossification of the posterior longitudinal ligament (OPLL), ankylosing spondylitis, Klippel-Feil syndrome, and congenital stenosis, as well as other rare conditions.

The pathology is also demonstrated in the sagittal view seen in FIG. 5 B, which shows multilevel disease often seen; furthermore, this is universally present with entities such as OPLL which was referenced above. One notes the posterior compression 180 caused by the combination of the thickened posterior longitudinal ligament, calcified disc herniations and osteophytes along the posterior border of the vertebral bodies of C3 to C6 102-105, narrowing the spinal canal 156 and compressing the spinal cord 175. In fact, it can be seen that at C6, there is a speckled region within the spinal cord 181 representing an area of injury to the substance of the spinal cord itself. The laminoplasty procedure which is the focus of this invention accomplishes relief of this compression, and in this case, preventing further injury to the spinal cord.

### Detailed Description of the Drawings Demonstrating the Invention

The invention is best understood by studying the following detailed descriptions in conjunction with the context of the accompanying images, wherein like reference numbers refer to like structures, in accordance with common practice. Also, in accordance with common practice, the structures illustrated are not necessarily drawn to scale, nor can inferences of scale be developed with respect to such drawings. The embodiments presented, and illustrations herein are general representations of the invention, and are not nor can they be construed to be restrictive.

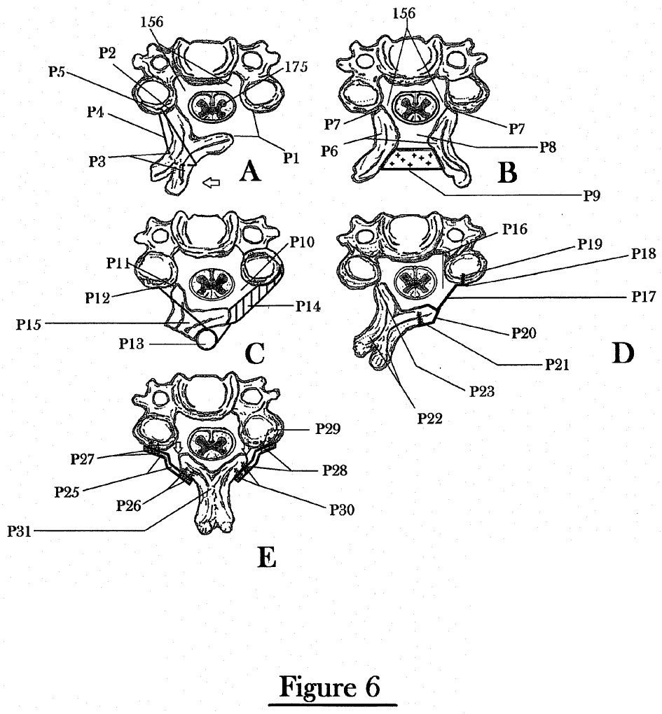

The many objectives of this invention can also be better understood by reviewing illustrations of representative previous art, shown here in FIGS. 6A-E. In the first image, FIG. 6A, one sees a variation of the original procedure as described by Hirobayshi. In this operation, the surgeon accomplishes a lateral osteotomy on one side P1 and, furthermore, a limited osteotomy on the other side P2. This is sometimes referred to as a “single hinged door,” procedure. This rotates the posterior arch of bone P3 to one side (arrow), and in this way the spinal canal 156 is enlarged, thus decompressing the spinal cord proper as well as the nerve roots (not seen in this view). In Hirabayshi's original description, the bone was rotated to the side and simply left loosely in place. Subsequent authors have added adjuncts such as the tether P4 seen herein, preventing the free bone arch P3 from rotating back into position and hence possibly leading to re-stenosis. The tether P4 is typically a wire, but can also be a heavy, nonabsorbable suture. It is passed circumferentially around the spinous process, and then attached to the posterior aspect of the lateral mass P5 contralateral to the osteotomy P1.

FIG. 6B presents a method proposed by Kurosawa in which the asymmetry sometimes seen in the original method shown in 6 A. In this method, the spinous process P6 is divided in the midline, which, along with lateral releasing osteotomies P7, create the “double-hinge,” design, which provides generous midline decompression P8 of the spinal canal 156. This splaying of the spinous process is maintained by a spacer P9, usually fabricated of metal, but in some instances a bone graft is implanted which matures to a restructuring of the posterior elements.

FIG. 6C displays the technique taught by Cathro in U.S. Pat. No. 6,080,157. This again utilizes a “single hinge door,” technique, with a large unilateral osteotomy P10 and a limited contralateral osteotomy P11, followed by securing a tether P12 to the lateral mass contralateral to the major osteotomy P10. The tether P12 is connected to a retainer P13 which is positioned between the spinous processes. The retainer P13 is, in turn, connected to a spacer P14. After accomplishing the osteotomies P10, P11 and securing the tether P12, traction on the tether 12 compels the free posterior bone flap P15 to be rotated and create a significant decompressive enlargement of the canal P10. The spacer P14 is configured to maintain the bone flap in the operative position and is therefore inserted into the defect created by the rotation of the hinge, as displayed in this image. This is ideal for a multilevel laminoplasty. The spacer P14 is actually plate-like as seen from a lateral perspective and is designed to span multiple levels. In this view, however, it is seen “end on,” such that the viewer does not appreciate its multilevel utility.

Another variation of a “single hinge door,” technique, as taught by Taylor in U.S. Pat. No. 8,172,875 and demonstrated in FIG. 6D, again involves a somewhat larger osteotomy on one side P16 with a relaxing osteotomy P23 on the contralateral side. In this embodiment, a fixation plate P17 maintains the position of the bone flap P22 after it has been rotated to decompress the neural elements. This plate is monolithic and is provided with an angled end P18 which is configured to appose the dorsal surface of the lateral mass. This segment of the plate is secured into position with small screws P19, not to be confused with standard “lateral mass” screws. The other end P20 of the plate is bifurcated and configured to be secured by screws P21 against the free edge of the bone flap.

In FIG. 6E, a variation as taught by Robinson in U.S. Pat. No. 9,107,708 can be seen. This art utilizes bilateral fixation plates P25, P28 which are secured by screws P27, P29 to the lateral masses bilaterally, as well as screws P26, P30 to a free bone flap 31 which is symmetrically elevated, decompressing the spinal canal.

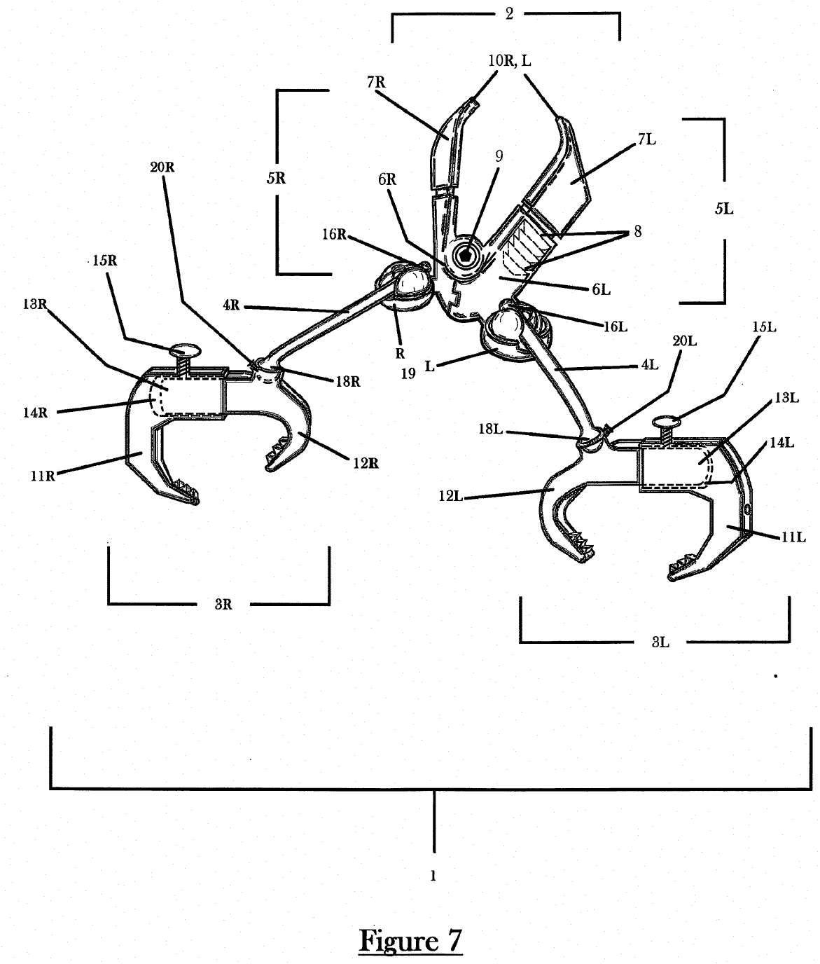

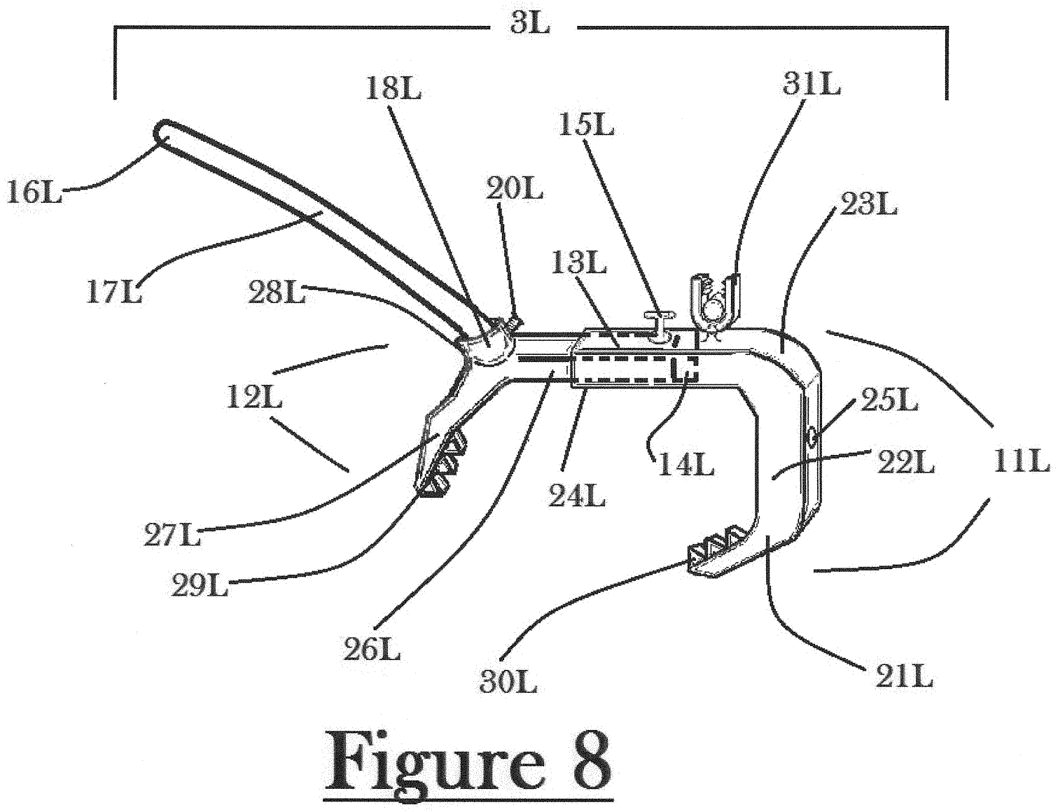

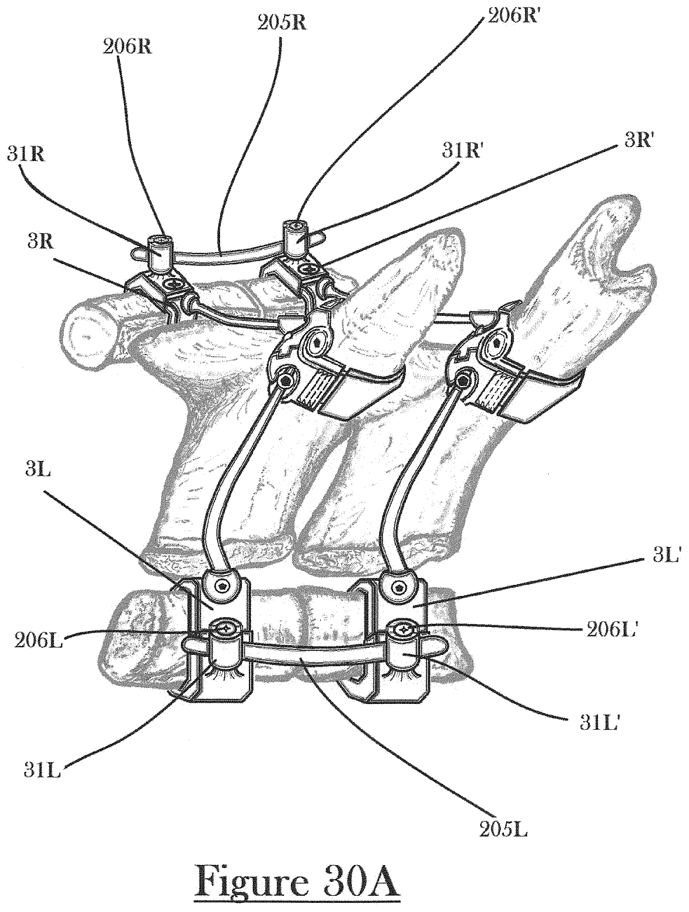

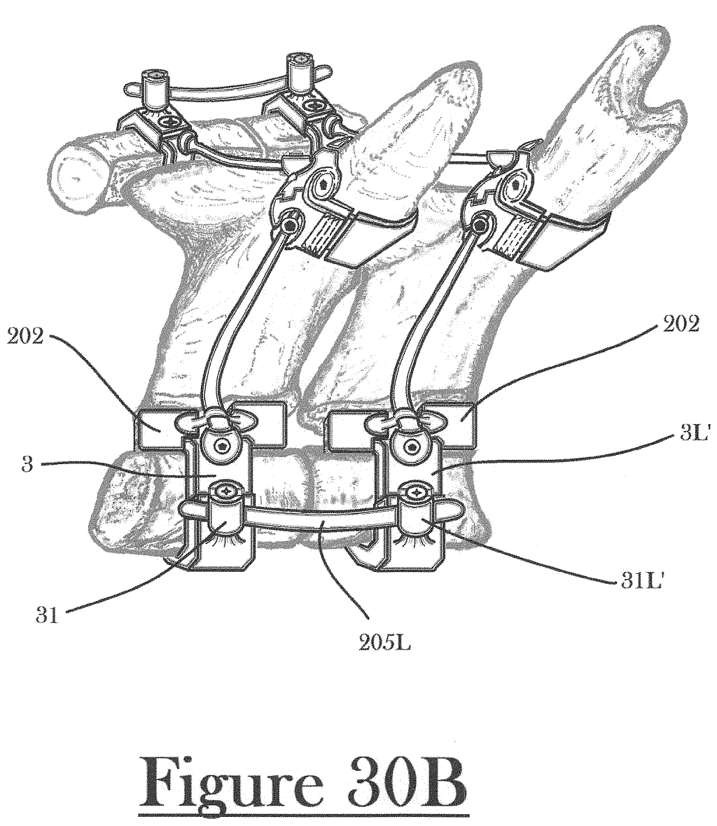

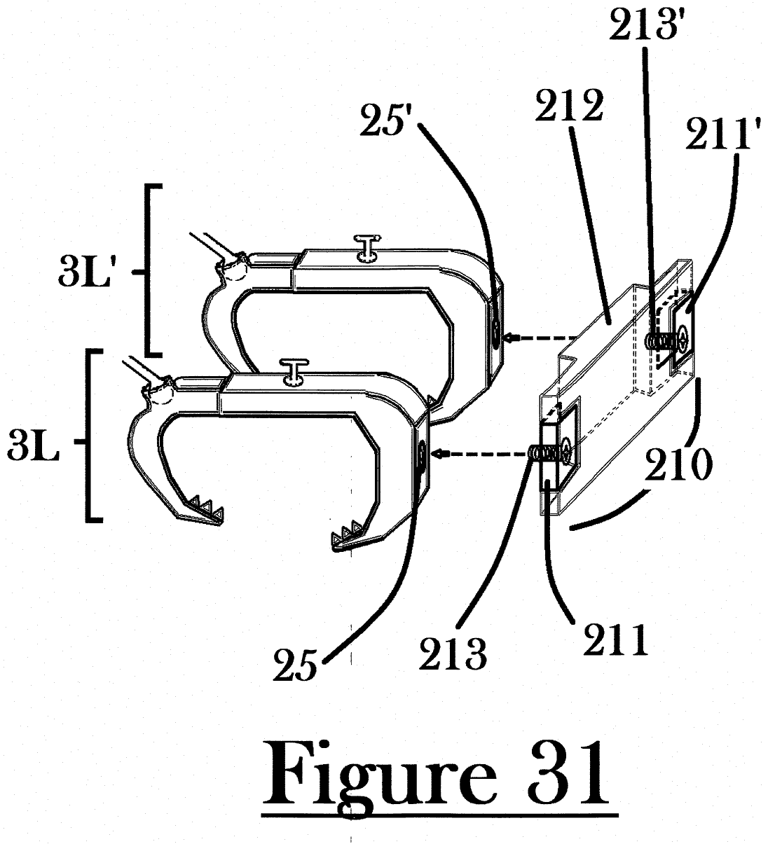

Having reviewed the pertinent anatomy and prior art the present invention can be fully appreciated, attention is turned to FIG. 7, an elevational view of the assembled preferred embodiment of the CMIL 1, in which there is a central, clamp-like Spinous Anchor 2 that is coupled via elevating stabilizers 4R, 4L to lateral anchors 3R, 3L. The spinous anchor 2 is comprised of left 5L and right 5R members which are in turn comprised of cranial elements 6R, 6L and caudal elements 7R, 7L, which are slidably, repositionably coupled to each other, providing adjustability in the craniocaudal dimension; adjustability in this dimension is important as the sizes of spinous processes will vary, and this will provide the maximum hold against the target bony areas. This coupling can be secured by a ratcheting mechanism 8L, which is shown in relief portraying the left sided mechanism in this image; of course, this repositionable coupling can also be governed by any other mechanism known or acceptable to the art, as reviewed below in FIGS. 30-35 below. Additionally, it is noted that in this embodiment, the cranial aspect of the cranial elements 6R, 6L are coupled by a securing screw 9 creating a mechanism by which members 5R, 5L are rotated around this pivot point until the ideal positioning against the target bony surfaces is accomplished; at that point, the screw 9 is secured in position. The caudal most aspects 10R, L of the caudal elements 7R, L are curvilinear in configuration, designed to be brought against the caudal aspect of the spinous process such that when the cranial 6 and caudal 7 elements of the members 5 are slidably compelled towards each other, the [curved] caudal ends 10R, L capture the caudal aspect of the spinous process, thus further increasing the grasp against the target bony surfaces. Multiple embodiments of the spinous anchor can be anticipated, some of which will be illustrated below; all such embodiments would be within the spirit and scope of the invention.

The lateral mass anchors 3R, 3L are likewise each comprised of two elements, a lateral element 11R, 11L and a medial element 12R, 12L, which are slidably coupled to each other, wherein the coupling ends 13R, 13L of the medial elements 12R, 12L are repositionable within chambers 14R, L provided to the coupling ends of the lateral elements 11R, 11L. This adjustability is critical in order to affix the lateral mass anchors 3R, 3L properly against the lateral masses, an attachment upon which the function of the entire system hinges. Upon achieving precise positioning, the lateral 11R,L and medial 12R, L elements of the anchors are locked by the lateral screws 15R, L, shown here is relief to demonstrate the tightening mechanism.

Two rod-like elevating stabilizers 4R, 4L are irreversibly coupled bilaterally to the medial elements 12R, L of the lateral mass anchors 3R, 3L, and are components of these anchors at the time of implantation; this irreversible coupling will be illustrated in subsequent drawings. The leading ends 16R, L of the elevating stabilizers 4R, L are then coupled to a receiving area within housing mechanisms 19R, L provided to the spinous process anchor 2, completing the construct. The trailing ends 18R, L of the elevating stabilizers 4R, 4L are spherical, which, upon coupling, provides adjustment of the construct in the anteroposterior dimension. This adjustability is integral to this coupling, which is then actuated, hence elevating the spinous process anchor 2 and with it the spinolaminar arch; this action enlarged the spinal canal in the anteroposterior dimension, achieving the principal goal of the surgical procedure. Upon completing this elevation, the position is locked with securing the stabilizing locking screws 15R, 15L.

The lateral mass anchors 3R, L are configured to be securely brought against the the medial, lateral, and posterior surfaces of the lateral masses. A frontal elevational view of the a left-sided anchor 3L is displayed in FIG. 8, where it can be seen that the anchor 3L is comprised of two elements, a lateral element 11L and a medial element 12L. These elements are configured to be secured against the lateral masses, presumably at or near the midposition of the craniocaudal dimension of the target lateral mass.

The lateral element 11L is noted to have a substantially horizontal segment 24L, a curvilinear segment 23L, a substantially vertically oriented segment 22L, and a leadingmost end 21L which is provided with limited teeth, 30L, which are primarily designed to increase friction. It is noted that these teeth may or may not be configured to violate the cortical surface. It is noted that there is an aperture 25L positioned on the vertical segment 22L, this aperture primarily serving to anchor pre-formed bone graft cartridges, which will be disclosed in greater detail below. Additionally, seen is the Lateral Screw 15, which is disposed through an aperture (not shown) in the horizontal segment 24L of the Lateral Element 11L, ultimately locking the Medial Element 12L with the Lateral Element 11L and securing the mediolateral dimension of the Lateral Anchor 3L. Also noted mounted to the dorsal aspect of the horizontal segment 24L is a cradle 31L which is configured to secure a rod extending over multiple levels the the surgeon has determined that a posterior fusion enhanced by stabilization is indicated, in addition to the laminoplasty. This will be discussed in greater detail below.

The medial element 12L is provided with a thin, curvilinear, plate—like engaging leading end, the leadingmost segment 27L having been provided with small, tooth-like projections 29L which are configured to increase friction and hence the securement of the anchors to the target bony surfaces. These teeth 29L may or may not be configured to penetrate the cortical surface of the bone. The engaging end 27L of the medial element 12L is configured to be positioned so that there is minimal intrusion upon the spinal canal upon placement of the medial element against the target bony areas; it is substantially curvilinear in the frontal profile, configured so as to closely conform to the transaxial profile of the [post-osteotomy] medial aspect of the lateral mass. Of note, in one embodiment, the [toothed] leading end 27L of the element 12L is configured to be disposed through a medial osteotomy created by a purpose-specific instrument so that there is no encroachment of the spinal canal in the mediolateral diameter; in another embodiment, this leading end 27L is configured to insinuate beneath the widest aspect of the bony prominence, optimizing the apposition of the anchor against the cortical surface which, in turn, optimizes the security of the element. Additionally, this image shows that the medial element 12L is also provided with a cradle 28L into which the trailing end 18L of the elevating stabilizer 4L is accommodated. This is secured with screw 20L. The medial element 12L is also provided with a horizontal segment 26L.

Each of these two elements are also provided with trailing ends configured to slidably couple with each other. This is achieved by bringing the lateral element against lateral aspect of the lateral mass, accomplishing a significant amount of apposition with the target bony surface areas. Upon achieving a secure hold against the lateral mass, the elements are locked in position by actuating a lateral securing screw. In order to achieve another object of the invention, to be described later, the trailing ends of these screws are provided with an elongated axis.

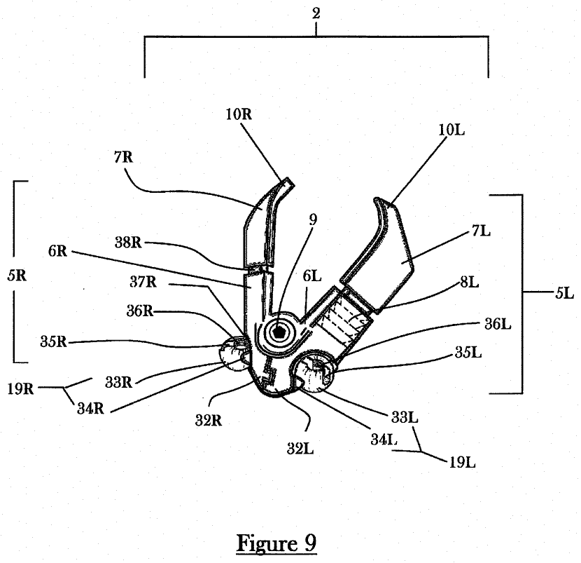

The spinous anchor 2 has multiple components and is substantially “U-shaped,” as viewed from a top perspective; this configuration is also appreciated in the elevational view in FIG. 9. The anchor 2 is composed of left 5L and right 5R sided members, coupled at their anteriormost 32R, L aspects such that each side is provided with arched mediolateral movement rotating around a central axis represented by a securing screw 9; this range of movement provides a secure fit regardless of the width of the target spinous processes. One takes note of the bias provided the entire Anchor 2, with the top, as seen in this image (posterior once implanted), is slightly more medial with the base (anterior once implanted), slanted outward, conforming to the general anatomy of an exemplary cervical spinous process. The members 5R, 5L are further configured with cranial elements 6R, L and caudal elements 7R, L which are slidably and [preferably] ratchetably coupled by extensions 38 from the cranial elements 6 to the caudal elements 7. The ratcheting mechanism 8L is shown in relief demonstrating that it is within the cranial element 6R. The caudal elements 7R, L are provided with curved components 10R, L that can be securely brought against the [curved] caudal aspect of the process; adjusting the anteroposterior axis assures a secure fit against the process, and this adjustment is locked by a ratcheting mechanism, a cold weld, a screw, a securing nut or any other means known or acceptable to the art.

Another structure seen in this image is the mechanism 19R, L to engage and lock the leading end of the Elevating Stabilizer. This mechanism 19 consists of a spherical receiver 33 which is positioned within a pair of arms 34; the manner by which the arms 34 secure the sphere 33 is nonrestrictive, permitting the sphere to rotate throughout a full turn in any direction. This would be necessary because the individual anatomy and degree to which the canal will be expanded can vary extremely amongst individuals. It is noted that there is a tract 35 within the sphere 33, said tract configured to receive the leading end of the stabilizer. Furthermore, there is also a tract 36 which provides a screw hole for a securing screw (not shown) to secure the leading end within the sphere once a satisfactory position is achieved. One notes, particularly well seen on the right side, that the sphere is partially contained within a socket 37R, which provides further stability for this critical coupling. This socket is one embodiment. The mechanism, for example could also be extended out from the cranial element 6 by an arm; other configurations can also be anticipated, some of which will be illustrated below.

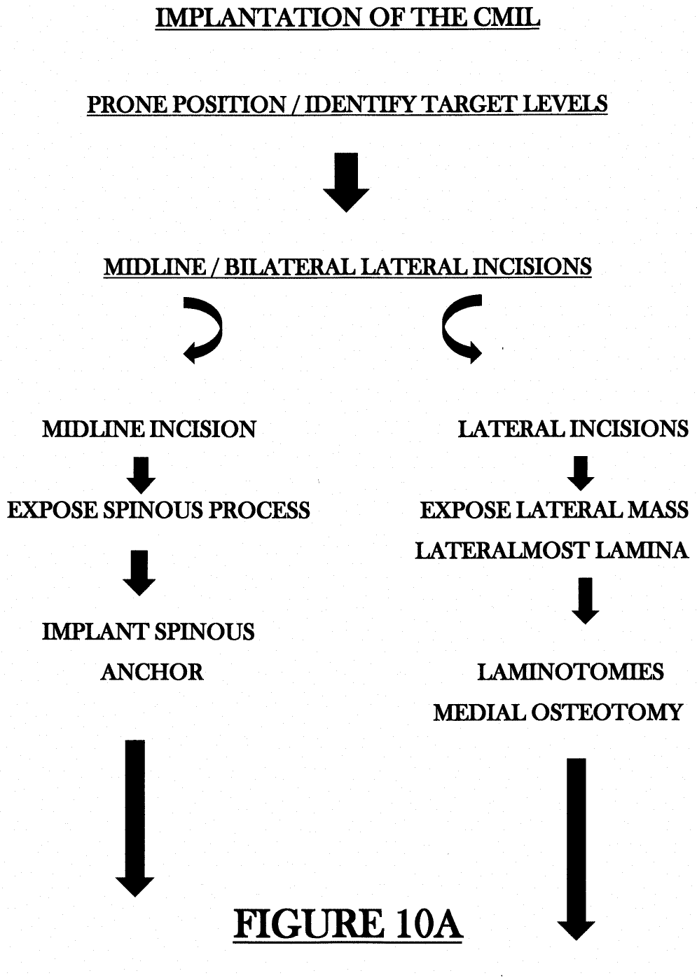

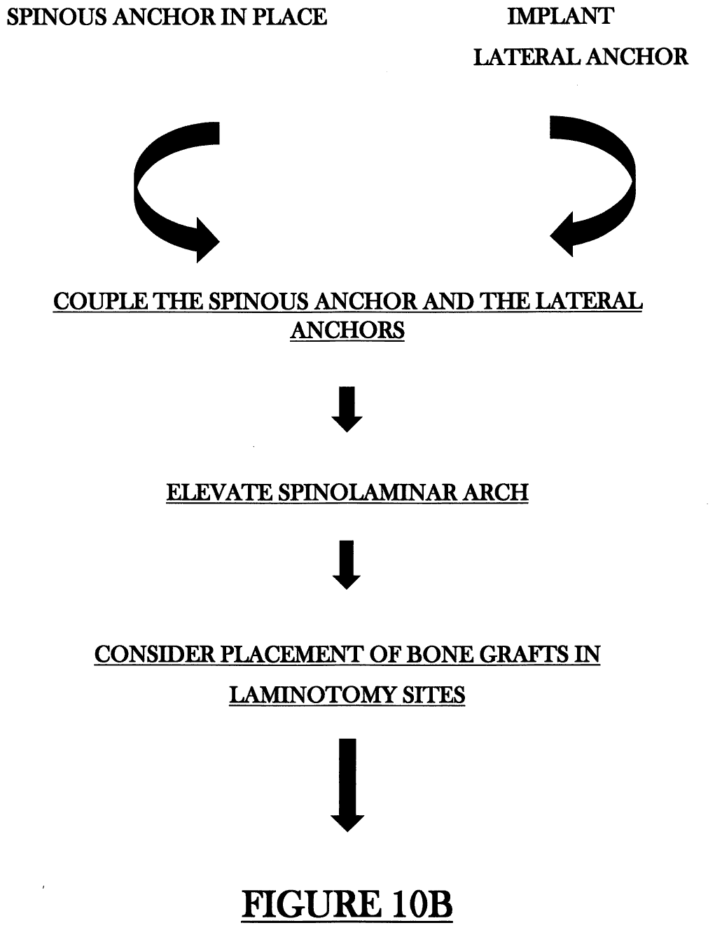

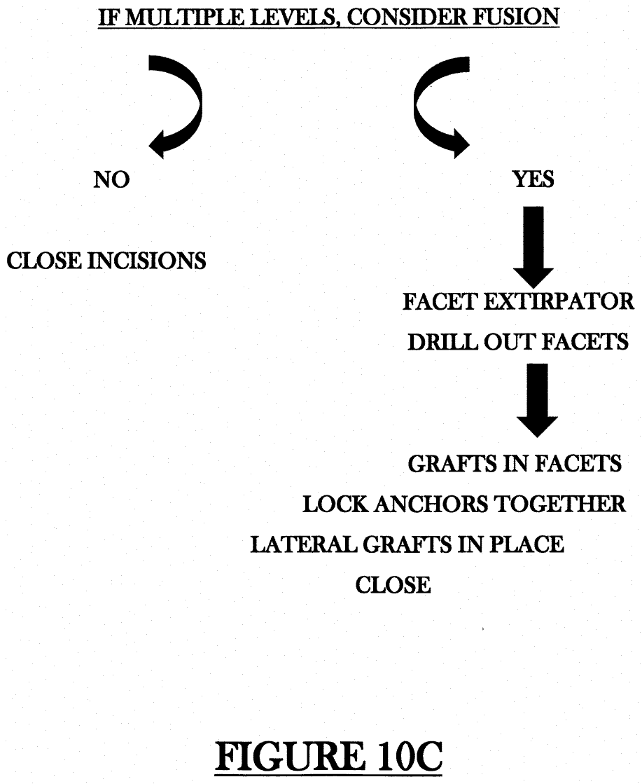

The implantation of the embodiment is discussed in “Flow Chart,” format in FIGS. 10A-C, a process that includes identifying the target levels and making appropriate incisions, exposing the target spinous process or processes and target lateral masses with retractors in place. At that point, the spinous anchor(s) is/are secured, and the laminotomy instrument is brought into the field, stabilized against the lateral mass and the appropriate laminotomies are made, as dictated by the software program. After utilizing the medial osteotome to create receiving areas for the medial components, the lateral anchors are seated, and coupling the lateral anchors to the spinous anchor is achieved; at that point, the spinolaminar arch is elevated. This can be accomplished through either of two strategies: in a first strategy, the spinolaminar arch is manually elevated, either before or after coupling the anchors; alternatively, the spinous anchor is coupled with the lateral anchors which are then utilized as fulcrums for the connecting elements therefrom serving as lever arms, then being actuated to elevate the spinous anchor/spinolaminar arch complex, decompressing the spinal canal.

The preferred embodiment couples the anchors initially. The connecting elements, known hereinafter as the elevating stabilizers, have a rod-like central segment which is monolithic with spherical trailing ends which are irreversibly encased within sockets provided to the medial elements of the lateral anchors. This configuration provides the elevating stabilizers with polyaxial movement capacity. The leading ends of the stabilizers couples with the spinous anchors; this coupling must also, in the preferred embodiment, be provided with polyaxial mobility, which is related to changes in the length of the elevating stabilizers utilized in this coupling as well as the change in the angle of this coupling. These dimensions are both highly variable related to individual anatomy, the extent that the surgeon elevates the spinolaminar arch, and the interaction of the anatomy to the CMIL. For these reasons, the angle of the receiving cradles cannot be “pre-set,” and, in the preferred embodiment, would disclose a spherical cradle that receives the engaging end of the elevating stabilizer.

Many surgeons prefer not to address the lateral osteotomies at the conclusion of the procedure, leaving these gaps to be eventually filled in by fibrous tissue; some of these surgeons believe that any attempts to promote bony regrowth can promote exuberant osteogenesis, resulting in the “restenosis,” or recurrence of the very pathology addressed by the operation; these speculations appear to be more theoretical than real.

Others believe that in time, a bony union will grow across the gap, although this tends to be wider—in many instances greater than 1 cm—than could be expected to fuse under most circumstances. The literature is unclear regarding this issue. Still other surgeons attempt to place some type of graft substrate in the hope of reconstructing the lamina or spinous process. Their position is that, in essence, “all hardware will fail in time,” and bony reconstruction is the only way to prevent such a hardware failure. This appears to be a very prudent and evidence-based position.

Therefore, the system disclosed herein offers the surgeon the option of implanting a pre-formed cadaveric bone graft into the bony defects resulting from the lateral laminotomies. In the preferred embodiment, this graft is irreversibly coupled to a metallic base which in turn is configured to be secured to the Elevating Stabilizer and/or the lateral anchors, all of which will be more completely disclosed below.

A single level laminoplasty would address the posterior elements of a single vertebra, and of course is performed as deemed by the surgeon. However, in the majority of instances, multilevel laminoplasties are necessary, as cervical stenosis is often multilevel. In pathologies such as DISH disease, it may involve all of the cervical and even the upper thoracic spine.

In cases where multilevel laminoplasties are performed, some surgeons prefer to simultaneously perform a fusion, often augmenting this with lateral mass screws and plates of rods. In this system, the lateral mass anchors can be easily coupled stabilizing the construct. Utilizing the elongated trailing ends of the Stabilizing screws, coupling plates are introduced, these plates provided with apertures configured to allow the trailing ends of the screws to be disposed therethrough in one orientation, but not orthogonally. After securing the plates against the lateral mass anchors of the target levels, the screws are rotated 90° thus locking the plates in place. Hence, stabilization, typically with fusion, of any number of levels can be accomplished. Embodiments of rods are provided which can be used in lieu of plates.

In conjunction with performing this fusion, the surgeon may choose to decorticate the facets associated with the levels to be fused. To this end, the surgeon may again choose to drill out the facet joints “freehand;” alternatively, a multi-purpose instrument is provided by which the surgeon can decorticate the facet joints and create a cavity entirely within cancellous bone of both the cranial and caudal lateral masses.

This device, hereinafter known as the facet extirpator, includes a leading end and a trailing end, which have been coupled by a central connecting shaft. The leading end includes a plate which is brought against the lateral mass anchors to center the device, and a drill which is dimensioned to create a cavity into which a prefabricated cadaveric bone graft can be inserted to promote facet, and hence posterior fusion. The central connecting shaft couples the trailing end, which is provided with a rotatable handle by which the surgeon actuates the drill, to the leading end of the instrument, whereupon a gear housing mechanism is encased; furthermore, the central shaft also provides a means by which the surgeon can stabilize the extractor while drilling. As an adjunct to this device, the system also offers a bone graft that can be placed into the cavity created by the facet extirpation. Specifically, a cadaveric graft is fashioned to comport to the dimensions of a cavity created by the drill at the leading end of the facet extractor. This graft can then be impacted into the cavity, while a brace coupled to the trailing end of the graft can be secured to the plate/rod that is coupling the lateral anchors, as described above. These features allow the system to achieve stabilization and fusion. Optionally, the facets to be fused can be distracted utilizing purpose-specific device which distracts the facet prior to drilling and extirpating the facet joint. Prior to distraction, an initial extirpation of the posteriormost aspect of the facet is achieved with a standard facet extirpator; this will leave a slightly narrowed “lip of bone,” which will retain a larger graft within the cavity. This creates an entry point which, when combined with distraction provides access for a larger extirpator which creates a larger central cavity. A specifically configured larger fusion graft is then insinuated within the larger cavity, and upon relaxing the distraction, the specifically configured oversized graft is encased within the facet joint cavity, thus applying an element of distraction without itself being at risk for spontaneous expulsion owing to the posterior lip of bone.

When performing a posterior cervical fusion, many surgeons will place bone graft substrate in the submuscular plane lateral to the lateral masses, euphemistically referred to by surgeons as the “lateral gutters.” This can be uniquely achieved with this system, while still maintaining the principles of “Minimally Invasive Surgery.” In order to do so, it is noted that the lateral plates of the lateral anchors are provided with one or more apertures which are in fact screw holes. After stabilizing the system, should a surgeon choose, disclosed herein is a cadaveric bone graft configured to be a flattened graft brought against the lateral aspects of the facet column. This graft is provided with [presumably] metallic cradles at the cranial and caudal ends, each of these cradles having a pre-loaded screw that can secure the graft to the screw holes provided to the lateral plates of the lateral anchors. The bone graft is thus held in place during maturation of the fusion. Optionally, hooks can be provided to the lateral plates of the lateral anchors, these hooks then providing a capture point for features provided to the metallic cradles of the lateral gutter grafts.

A final unique, useful, novel and nonobvious feature of this disclosure addresses the gap created by the osteotomies. This is generally left unattended, but there are some surgeons who prefer to utilize graft substrate to ultimately bridge this gap. The system therefore offers an option of a cadaveric bone graft which is configured to fit into that gap, the graft coupled with a bracket which is designed to be pressure fitted onto the elevating stabilizer, thus holding the graft in position during fusion maturation.

All the Pre-formed grafts disclosed in this specification, including those bridging from level to level, those occupying the cavities within the facet joints, and those occupying the spaces between the lamina and lateral masses may or may not include metallic brackets or cradles. Moreover, brackets/cradles which are comprised of other materials including absorbable materials, those in which the brackets/cradles are part of the preformed graft, and those in which there is no such bracket/cradle all represent the spirit and scope of the invention and therefore are incorporated within the scope of this application.

Selection of the incision sites is critical in maintaining this as a “Minimally intrusive,” procedure. As one of the goals of this procedure is to maintain a significant amount of musculature attached to the spinolaminar arch after it is elevated, this goal is best reached by creating midline incisions that are limited in scope and centered directly over the midportion of the target spinous processes; additionally, the lateral incisions should be also limited in scope, and centered over the lateral third of the lateral mass. Many surgeons would be most comfortable choosing their own incision sites, which is most acceptable provided they adhere to the guidelines set forth herein. As an alternative, offered herein is a guide template which assists the surgeon in further maintaining these goals.

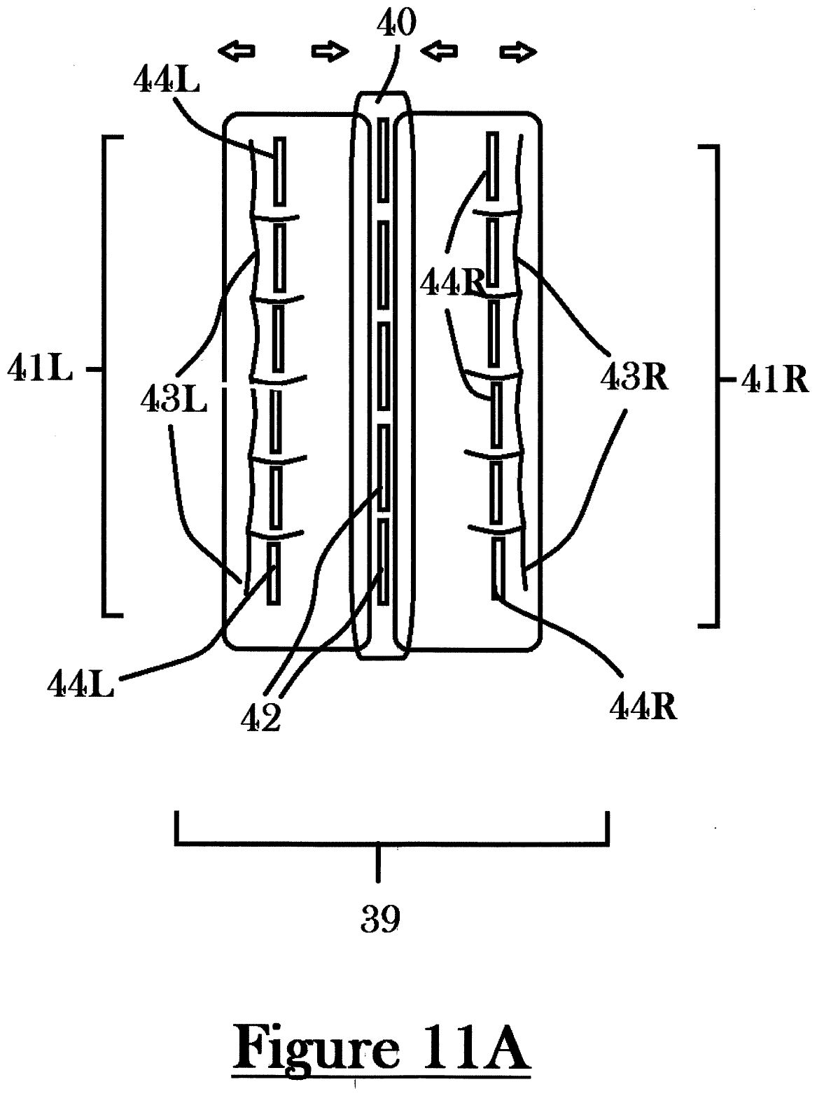

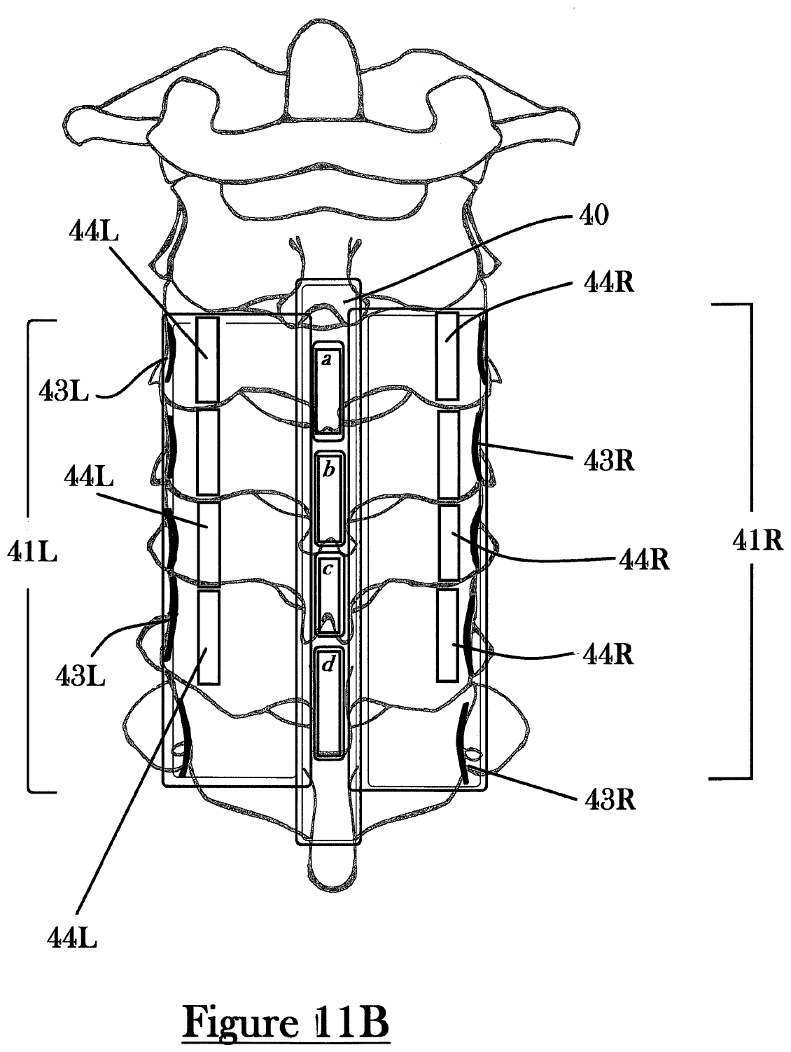

This guide template is demonstrated in FIG. 11A, wherein one can see that this template 39 is comprised of a midline component 40 and lateral components 41R, L which are slidably coupled with each other. All three components are impregnated with radiopaque markings that guide the template 39. The midline component 40 is provided with a series of vertically oriented, linear apertures 42, the outlines of which are impregnated with a radiopaque substance that allows the surgeon, with the use of Fluoroscopy, to identify the spinous processes and where these align with the apertures on the surface. With some effort, the apertures can be aligned to the midline of the spinous processes. The apertures dictate the position of the midline skin incisions, as well as the size of the incisions. In the setting of single level laminoplasty, a single level incision is needed; the template has been developed to specifically developed to dictate the most efficient incision, so that the spirit of the minimally intrusive surgical procedure can be maintained. In the setting of a multilevel procedure, individual midline incisions are still recommended, although at the surgeon's discretion, these can be conjoined to create a single, longer incision.

The lateral incisions are performed in accordance with the data derived from the lateral components 41R, L of the template. These have been impregnated with a radiopaque substance, which is also visible to the eye; the configuration 43 of the of this line generally represents the configuration of the lateral profile of the cervical spine, as seen from the posterior view. Once the midline template is set, the lateral templates can be moved in the mediolateral direction (as indicated by the open arrows at the top end of the template) until the radiopaque marker is aligned with the fluoroscopic appearance of the lateral profile of the spine. When these have been aligned, the apertures 44R, L would direct incisions over the lateral third of the lateral mass, which would provide the necessary exposure to the lateral mass while being minimally intrusive to the musculature attached to the spinolaminar arch.

The manner in which this guide template 39 functions is illuminated in FIG. 11B, wherein it can be seen that the template, which in the preferred embodiment is a sterile, consumable article, is brought into the operative field once the patient's skin has been prepared and draped. The template 39 is placed on the skin and Posterior-anterior projection fluoroscopic views are obtained. This image shows the template cast against the bony outline of the cervical spine. The midline component 40 is first aligned with the midpoint of the cervical spine. Although this usually correlates with the midline of the spinous processes, this may not always be true as they can be slightly rotated, and therefore great care must be taken to assure that “midline,” is the midposition between the lateral edges of the spine. The midline component 40 is provided with a series of apertures 42a-d, the apertures being denoted by the letters only to maintain clarity of the image. The perimeters of these apertures are outlined with a radiopaque substance to assist with alignment, and the apertures are so configured and positioned such that when the template is properly aligned, incisions can be guided by these apertures, with such incisions providing access to the cranial face of the target spinous process; this would be in accordance with the preferred embodiment. However, it is to be recalled that other embodiments of the spinous process are conceivable and included within the spirit and scope of the invention; included in these alternative embodiments are embodiments in which the caudal surface of the spinous process is the primary target bony area, and in such an embodiment, the template would be so configured so as to provide access primarily to the caudal face of the spinous process.

Once the midline has been unambiguously identified, then attention is turned to the lateral components 41R, L of the template. These are extended laterally until the lateral radiopaque markings 43R, L are aligned with the lateral edge of the fluoroscopic image of the spine. This positions the lateral apertures 44R, L such that incisions carried through these apertures will be made will be accomplished over the lateral mass in such a way as to provide access to the lateral aspect of the lateral mass as well as the junction of the lamina with the medial aspect of the lateral mass while preserving an ample attachment of the paracervical musculature to the dorsal surface of the target spinolaminar arches. In the setting of a multilevel procedure, multiple independent incisions can be made, both in terms of the midline incisions and the lateral incisions; alternatively, these can be represented by a single, longer incision, while still maintaining the spirit of a minimally intrusive procedure.

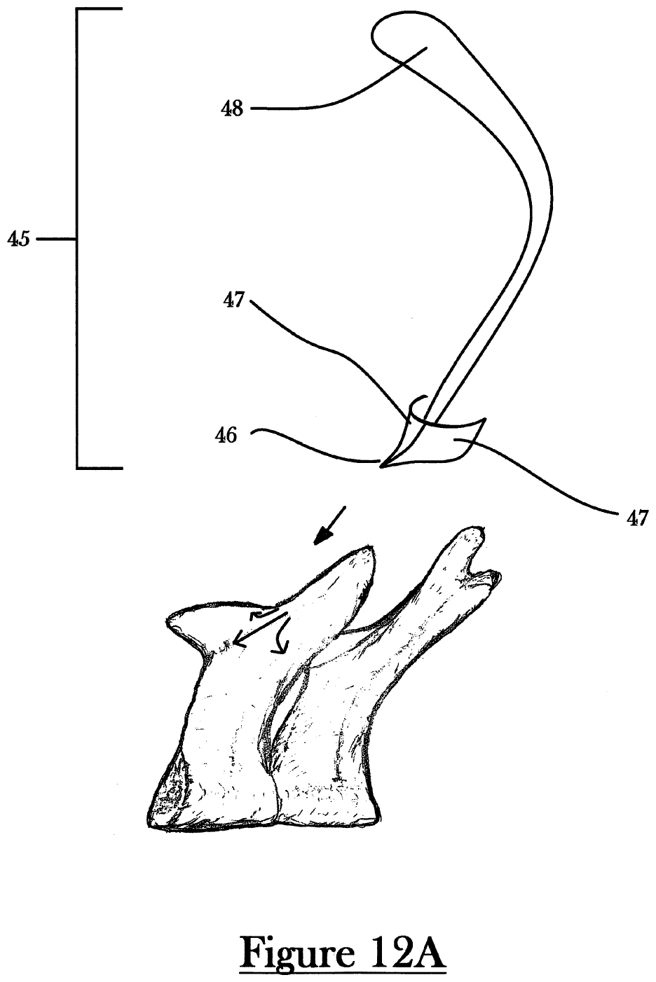

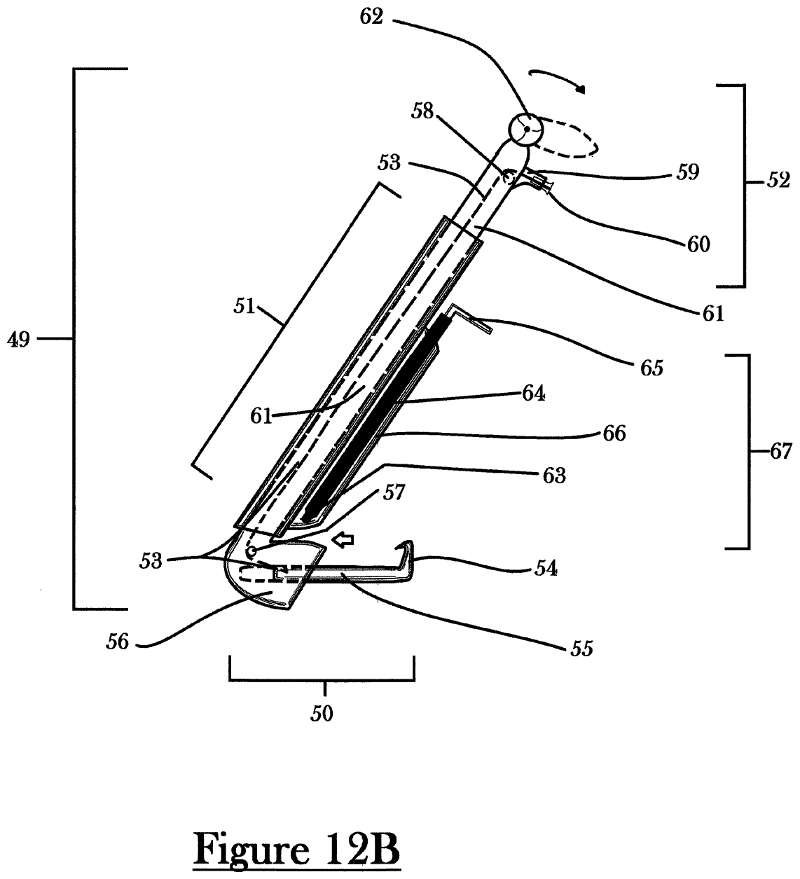

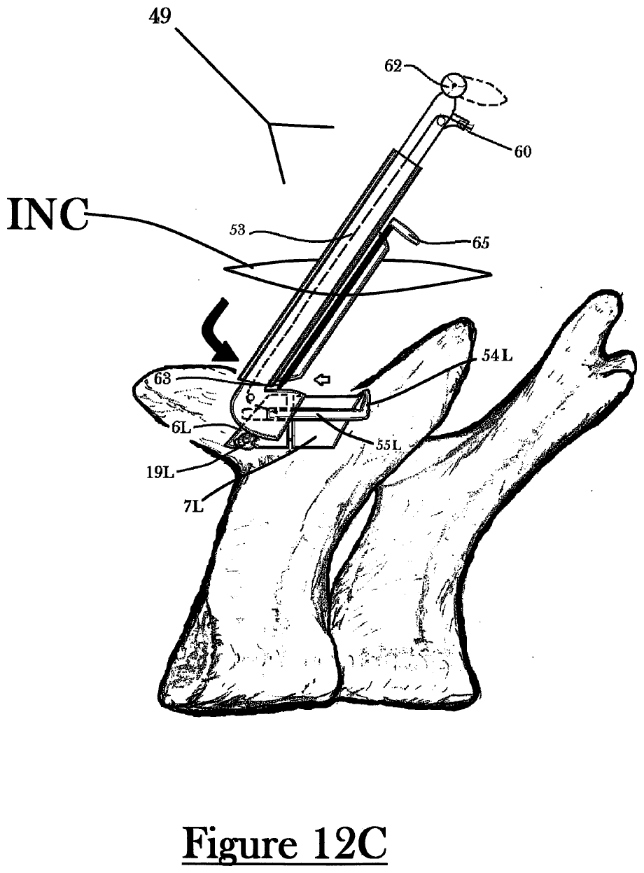

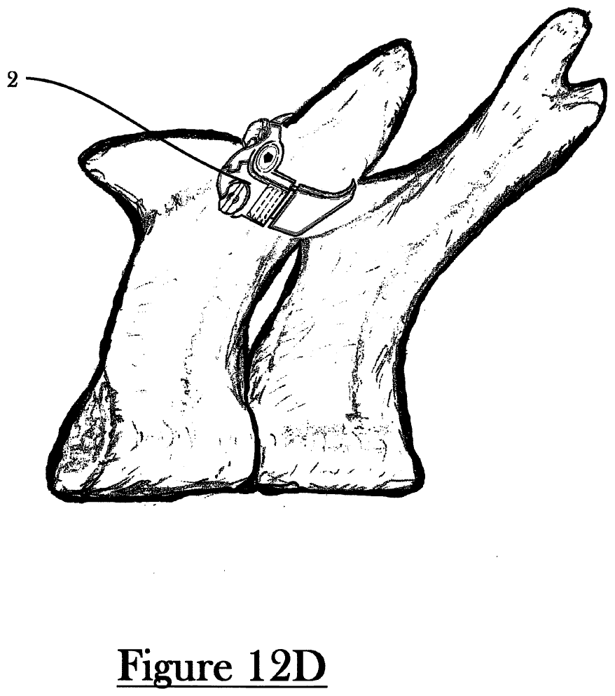

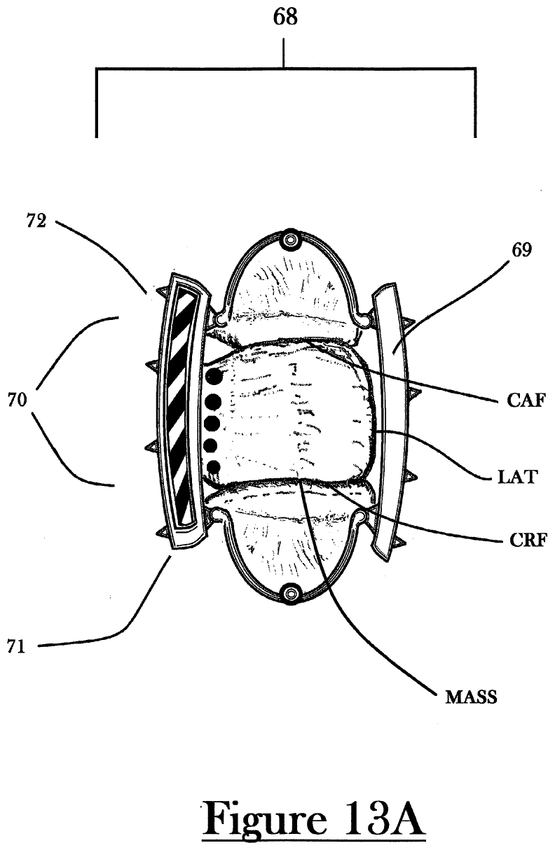

After the midline incision is created, the tip of the spinous process is identified, and the fascia released therefrom. A purpose-specific instrument 45 is introduced, as demonstrated in FIG. 12A. The leading end 46 of this dissector is carried through the operative field, as indicated by the larger solid arrow, against the cranial face of the target spinous process, and with gentle sweeping motion, a subperiosteal dissection is completed. The lateral wings 47 prepare the lateral aspects of the spinous process, as indicated by the smaller arrows, which is now ready to receive the spinous anchor. By leaving the musculature attached to the caudal aspect intact, the blood supply to the periosteum and hence the bone is maintained. The dissector is controlled by the trailing end 48 which acts as a handle, allowing the surgeon to manipulate the dissector 45.

After preparing the spinous process to accept the anchor 2, then an implantation tool is brought into the surgical field. This instrument 49, which is displayed in FIG. 12B, reversibly couples with the anchor and then is disposed through the incision whereby the anchor is implanted against the spinous process. The challenge in designing such an instrument is that it must accomplish three tasks: firstly, it will rotate the left and right members of the spinous anchor towards each other as the primary step in implantation; secondly, the instrument must compress the cranial and caudal elements of each member towards each other to secure these members against the caudal aspect of the spinous process, thus achieving a more secure capture of the target spinous process; thirdly, the instrument must tighten the midline securing screw which then locks the anchor in final position.

This instrument 49 is provided with bilateral leading ends 50 (the left side being demonstrated in this view), a central segment 51 which acts as a sheath containing bilateral central shafts 61 which connect the leading ends 50 to bilateral trailing or actuating ends 52 which serve as the actuating ends of the instrument 49. The leading ends 50 are represented by jaws which reversibly couple with the members of the spinous anchor. On each side, these jaws are themselves provided with a leading end 54 which couples with the caudal element of the [left as demonstrated] member of the spinous anchor, as will be illustrated in FIG. 12C. A rod 55 then extends from the leading end 54 into the expanded, shell-like structure 56 which houses the spinous anchor configured to reversibly couple with the cranial element of the member of the spinous anchor. Within this shell-like anchor housing is housed a cable 53, which may be fabricated from Nitinol or other “memory” metal; alternatively, this could be composed of a hard but flexible plastic, steel cable, or any other substance known or acceptable to the art. This cable 53 is irreversibly coupled to the rod 55, and after wrapping around the cable housing mechanism 57, the cable 53 is directed superiorly through a shaft-like configuration 61 housed within the central segment 51, as indicated by the interrupted lines, until the cable 53 is wrapped around another rotatable cable housing unit 58 near the trailing end of the central segment 51, exiting into a sideport 59, and terminating in an actuating pin 60. This complex of the cable 53 and the design of the leading end of the jaw is configured to compress the cranial and caudal elements of the members of the spinous anchor, as indicated by the open arrow above the jaw and which is integral to implantation. This configuration is in combination with another component feature of the instrument 49, which is the elongated shaft 61 extending from the trailing end 56 of the jaws 50. Such shaft-like configurations 61 extend from the leading ends 50 of the instrument 49, being contained within the central segment 51 of the instrument 49. This central segment 51 serves as a sheath providing a rotational degree of movement, which can be actuated by the trailing end 52 of the instrument 49. The trailing end 52 of each shaft 61 is provided with a rotatable handle 62; the exemplary illustration given here shows the handle 62 initially directed towards the reader; the rotation of the handle 62 into the plane of the paper is implied by the interrupted lines of its profile and the directional arrow above; any configuration which provides rotation to the leading end and hence the preferred embodiment of the anchor is within the spirit and scope of the invention. This action, when performed bilaterally, will rotate the left and right members of the preferred embodiment of the spinous anchor towards each other until the anchor has been firmly brought against the bony target surface areas. Once this is achieved, then the securing screw of the anchor is locked by another feature of the instrument 49, which is locking wrench 67 that is housed within a sheath 66 that is monolithic and continuous with the sheath 51 representing the central segment of the instrument 49. This wrench 67, which is illustrated in solid black, is provided with a leading end 63 configured to couple with the trailing end of the screw and provided with an Allen wrench, as shown here; it could alternatively be provided with a single ridge screwdriver, a Phillips head or any other configuration known or acceptable to the art. The wrench 67 is also provided with a shaft 64 and a trailing end 65 which is the actuating component of the wrench 67. Once the anchor is in ideal position against the spinous process, the leading end 63 is advanced into the head of the screw, the handle at the trailing end 65 is actuated, locking the screw.

At that point, the final action necessary to lock the anchor against the spinous process is to compress the caudal element of the members of the spinous anchor with the cranial end. This is accomplished by traction on the actuating pin 60 on the trailing end of the instrument 49. Pulling the pin 60 places traction on the cable 53, resulting in traction on the rod 55 and ultimately the leading end 54 of the jaw 50, which is again suggested by the open arrow. This, in turn, compresses the anchor contained within. The anchor would then be secured against the spinous process; after placement, the instrument 49 is gently rocked from side to side, freeing it so it can then be removed from the operative field. Rotation of the members of the anchor towards each other until secure against the spinous anchor is necessarily the first action in implanting the anchor; however, securing the screw as opposed to compressing the cranial and caudal elements do not have to be performed in any specific or proscribed order.

The actions of the instrument are illustrated in FIG. 12C, wherein a left lateral elevational view is offered which shows a spinous anchor, represented by the left-sided cranial 6L and caudal 7L members, reversibly coupled to the leading end of the insertion instrument 49. This leading end is seen being disposed through the incision INC and into the operative field. The curved, closed arrow shows the leading end of the instrument 49 being passed to the base of the spinous process and then rotated caudally. Actuating the trailing end 62 brings the two halves of the spinous anchor against the cranial face of the spinous process at which point the trailing end 65 of the wrench is actuated, causing the leading end 63 to tighten the screw that locks the two sides of the anchor. Then, the cable 53 responsible for compressing the caudal 7L and cranial 6L elements is actuated by pin 60, and the leading end 53 of the jaw is deployed, completing the positioning of the anchor. The housing mechanism 19 is now in position to couple with the elevating stabilizer. FIG. 12D shows the preferred embodiment of the spinous anchor 2 in final position.

After securing the spinous anchor, attention is turned to placement of the lateral anchors. Of course, the order of implanting the anchors disclosed herein cannot and should not be construed to be restrictive, and the lateral anchors can be implanted initially at the discretion of the surgeon.

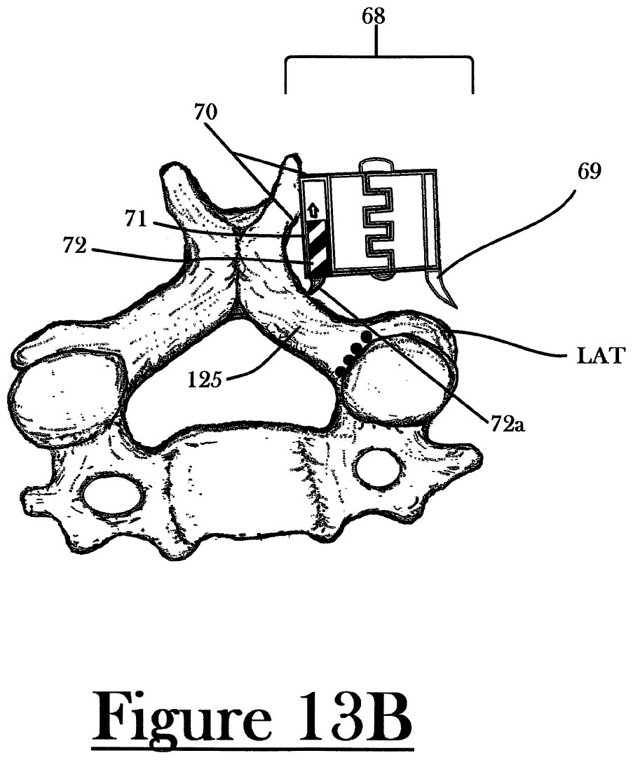

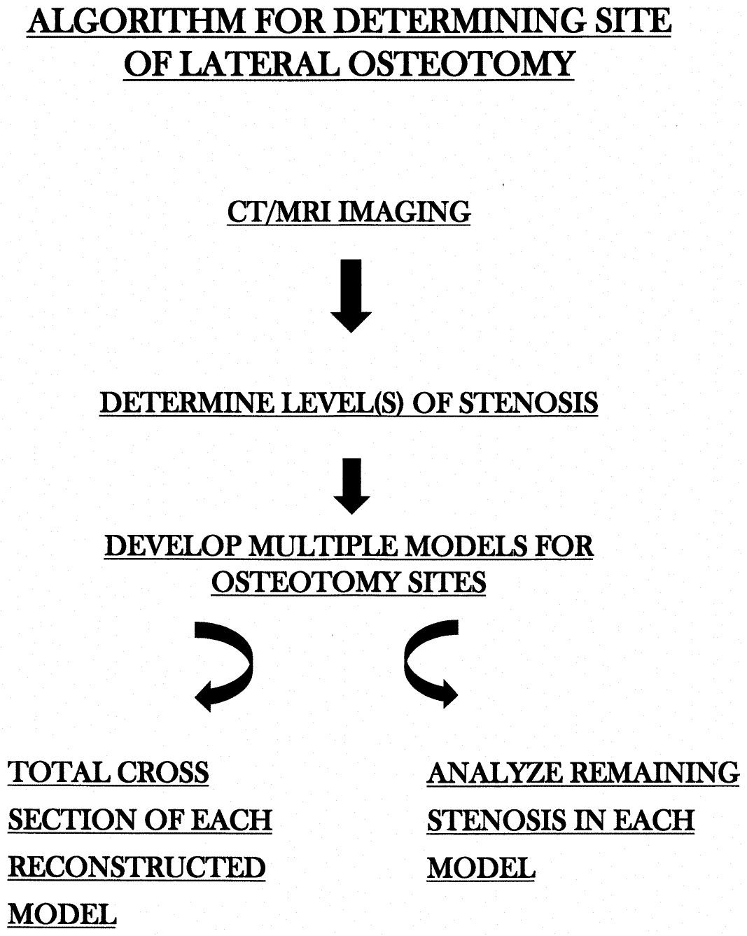

Just as with the spinous anchor, the first step in implanting the lateral anchor is to properly expose the target bony areas. After accomplishing an incision over the junction of the middle and lateral thirds of the dorsal lateral mass, the dorsal cervicothoracic fascia is opened and the muscle overlying the lateral mass is split. A surgeon's view of a left sided exposure is portrayed in FIG. 13A. With the dorsal aspect of the lateral mass MASS now in view, a unique retractor 68 provides for a lateral blade 69 which is positioned to expose the lateral edge LAT of the lateral mass. The unique complexity of the medial blade 70 relates to its design in which an outer blade 71 houses an inner blade 72 (stippled area) which is adjustable in the vertical dimension (anterior-posterior relating to the anatomy when positioned within the surgical field); this dimensional adjustment allows for the retractor to be positioned within this surgical field without being rotated by the bias of the cervical laminae. For orientation in this projection, the patient's head would be positioned at the bottom of the page, with the shoulders at the top. Hence, the retractor 68 can easily expose the proposed osteotomy site at the junction of the lateral mass with the lamina, represented by the vertical line of solid circles; the largest circles are at the caudal aspect of the lamina, which would be the site of establishing the osteotomy, which would be then carried cranially (towards the smaller circles). The cranial CRF and caudal CAF facet joints are also seen at the superior and inferior limits of the exposure.

As discussed, the medial blade 70 is designed in a purpose specific fashion to accommodate the biased contour of the lamina 125, which assumes a posterior bias along its course medially to join with the lamina from the other side to form the base of the spinous process. This is best seen in the transaxial view in FIG. 13B, where the medial blade 70 is provided with an outer frame 71 which houses a central blade 72 (stippled) that is adjustable in height in association with its position on the lamina 125. This adjustability is indicated by the arrow. As the retractor 68 is opened, the medial blade 70 is moved along the [angled] lamina 125, and the leading end 72a of the central blade 72 retracts into the outer frame 71; in this fashion, the retractor 68 remains balanced and is not twisted or rotated by the positioning. The lateral blade 69 is seen extending beyond the lateral aspect LAT of the lateral mass. The anticipated site of the osteotomy is indicated by the line of solid circles.

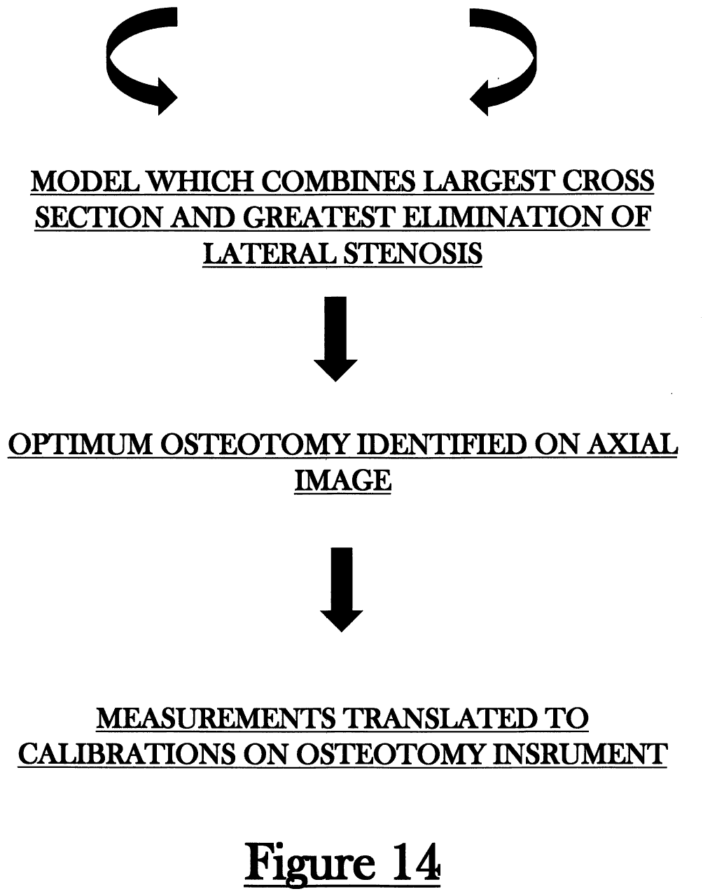

Determining to optimal site for the bilateral osteotomies, typically at the confluence of the lateralmost lamina with the medialmost aspect of the lateral anchor is also important in the execution of this procedure. The positions of these osteotomies can, of course, be estimated by the surgeon and achieved “freehand,” with instrumentation that the surgeon typically utilizes; alternatively, it is proposed herein that a unique software mapping program is utilized in association with a purpose-specific device known as the lateral guide and drill. This software program software program is designed to specifically analyzes the transaxial, sagittal and coronal images (CT or MM), creating ultimately a 3D model and analyzing a series of potential osteotomy sites and determining which of these would provide a maximum decompression. The program then provides a pixel/voxel registration along the line whereby an osteotomy should be performed to achieve this. Ideally, the lateral osteotomies would be brought through the medial aspect of the lateral mass and lateralmost aspect of the lamina, so that the osteotomy maximally enlarges the canal and achieves complete decompression of the neural elements. This determines that increase in total cross-sectional area of the spinal canal provided by a each of a series of proposed osteotomy sites. When performing such osteotomies too far medially, retained stenotic elements will promote continued compression along the lateral aspects of the canal—what is referred to (especially in the lumbar spine) as “lateral recess stenosis;” this process can also be seen in the cervical and upper thoracic spine, particularly, particularly after a decompressive laminectomy. Therefore, the software selects the optimal position of the osteotomy sites by evaluating which proposed osteotomy sites provide the maximum enlargement of the spinal canal while reducing or eliminating persisting lateral stenosis. By also analyzing the registration of the lateral and dorsal aspects of the lateral mass, which is where the lateral guide and drill is to be anchored, this series of data are coordinated with calibrations on the drill and guide which will dictate its settings. The osteotomy can therefore be achieved in the precise site dictated by analysis of the transaxial image. The algorithms used by the program are summarized in FIG. 14.

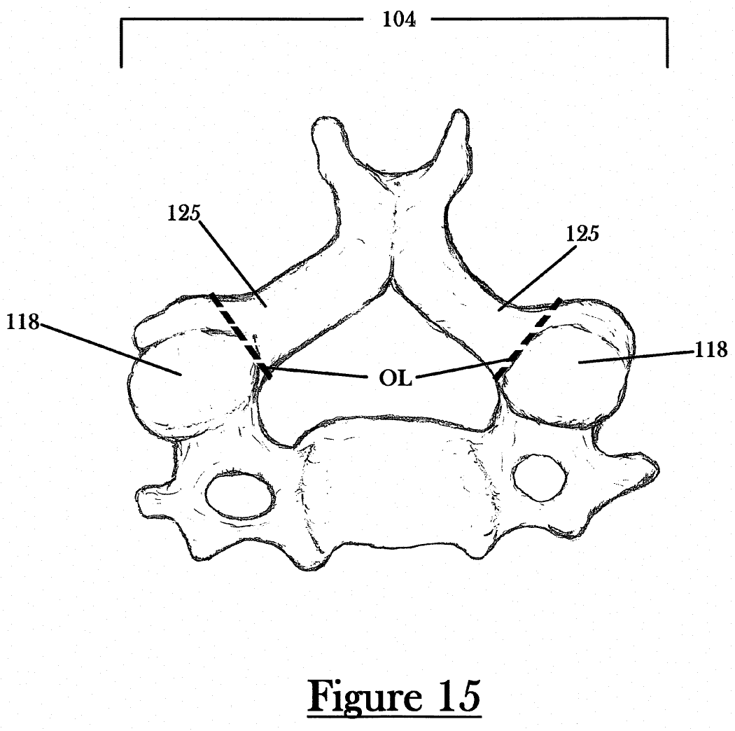

The practical application of these algorithms is demonstrated in FIG. 15, wherein the positions of the osteotomies are predicted by review of the transaxial view of the target vertebra. This transaxial image is of C5 104 as an exemplary vertebra, with the interrupted lines OL representing the proposed sites of the osteotomies. These are at the confluence of the lamina(e) 125 with the lateral masses 118. In the preferred embodiment, the most caudal image would be utilized, with the drill cradle then creating the initial osteotomy at the caudal edge of these structures—where the distance to the dura/spinal cord is the greatest owing to the biased profile of the lamina. The osteotomy is then carried cranially and carried deep until a thin shell of bone remains, which can then be removed with a standard rongeur.

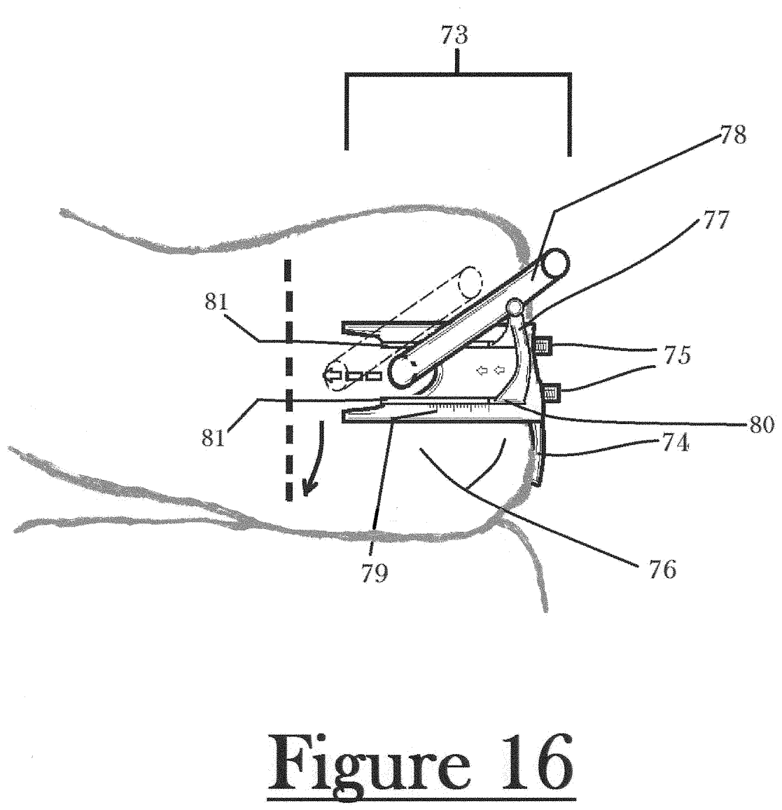

As disclosed above, the data developed from the algorithm in FIG. 14 is then translated to the lateral guide and drill 73, shown in a [right] lateral elevational view in FIG. 16. This instrument 73 is provided with a first, curvilinear base 74 which is positioned against the lateral aspect of the lateral mass. It can be held in position by a plurality of temporary screws 75, or manually by a handle provided to the surgeon (not shown in this image). The curvilinear base 74 is monolithic and continuous with a second, substantially flattened base 76 which is brought against the posterior aspect of the lateral mass, arising from which is a saddle 77 that stabilizes a central drill cradle 78. The base of the saddle is provided with footplates 80 which are slidably coupled with tracts 81 which have been provided to the flattened base 76, hence making this complex slidably adjustable along the mediolateral axis. The position of this drill cradle 78 can be slidably correlated with calibrations 79 of the flattened base 76. These calibrations specifically relate to positioning the cradle in accordance with the data as analyzed by the companion software program. Additionally, the angle of the cradle 78 as it relates to the plane of the posterior aspect of the lateral mass is also scored/calibrated in terms of this reference angle. The generation of these data points by the software program will also predict the position of a drill (not shown) within the cradle 78. The depth to which this drill will be actuated in order to achieve the desired osteotomy is a third data point provided by the program which will be disclosed below.

The actuation of the lateral guide and drill is implied in the superimposed image, noted in interrupted lines, which shows the position of the saddle 77 and drill cradle 78 having been repositioned (indicated by the open arrows) in accordance with the data generated by the software program. The site of the repositioned cradle is indicated by its “ghosted” outline created by the interrupted lines. As the entire craniocaudal dimension of the lamina must be osteotomized, an additional range of movement is provided to the cradle. This can be achieved either through providing a base which is as wide as the lamina; alternatively, the cradle 78 can be configured to allow the drill to be rotated in a craniocaudal axis, as in this example. The solid curved line shows the course the leading end of the guide in order to position the drill to commence the osteotomy at the most caudal end of the lamina. The course of the osteotomy is denoted by the interrupted line.

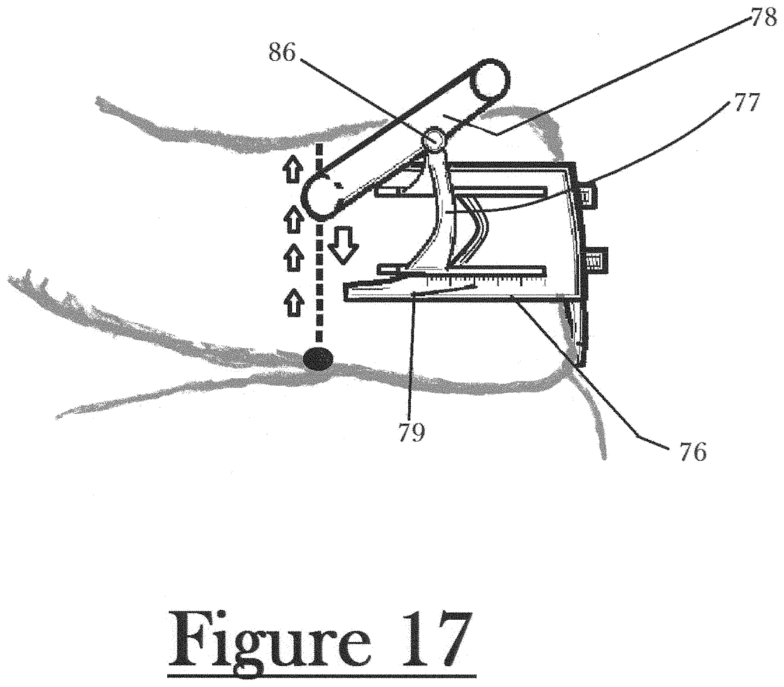

This is further demonstrated in FIG. 17, in which the saddle 77/cradle 78 complex has been slidably repositioned as directed by the calibrations 79 of the flattened base 76. The trailing end 86 of the saddle 77 is provided with a swiveling mechanism giving the cradle 78 a caudacranial range of motion, in addition to a protractor—like mechanism by which the angle of the cradle 78 with respect to the flattened base 76, and presumably the target bony areas, can also be pre-set in accordance with the software program. Again, the site of the proposed osteotomy is demarcated by the interrupted line. A single larger open arrow directed caudally indicates the repositioning of the angle of the cradle 78 so that the osteotomy can begin at the caudal end of the lamina, which is indicated by the solid oval. The osteotomy can be conducted from caudal to cranial aspects or vice-versa, although from the perspective of surgical anatomy, there may be some advantage to commencing caudally. The series of smaller open arrows indicate the direction the osteotomy would be carried.

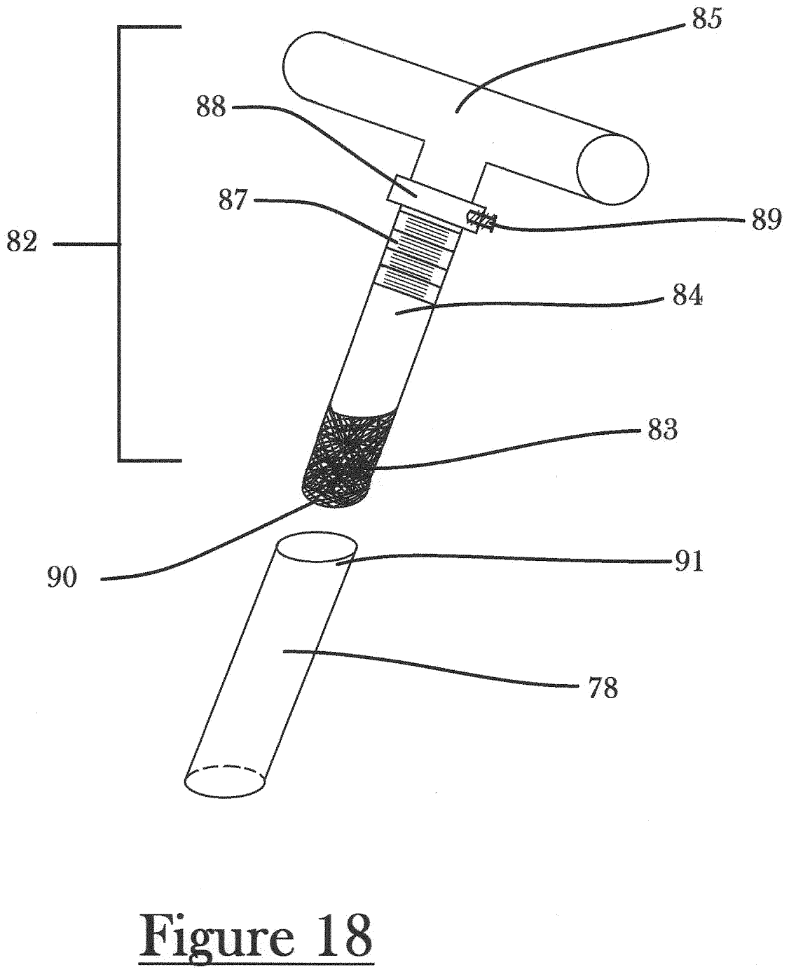

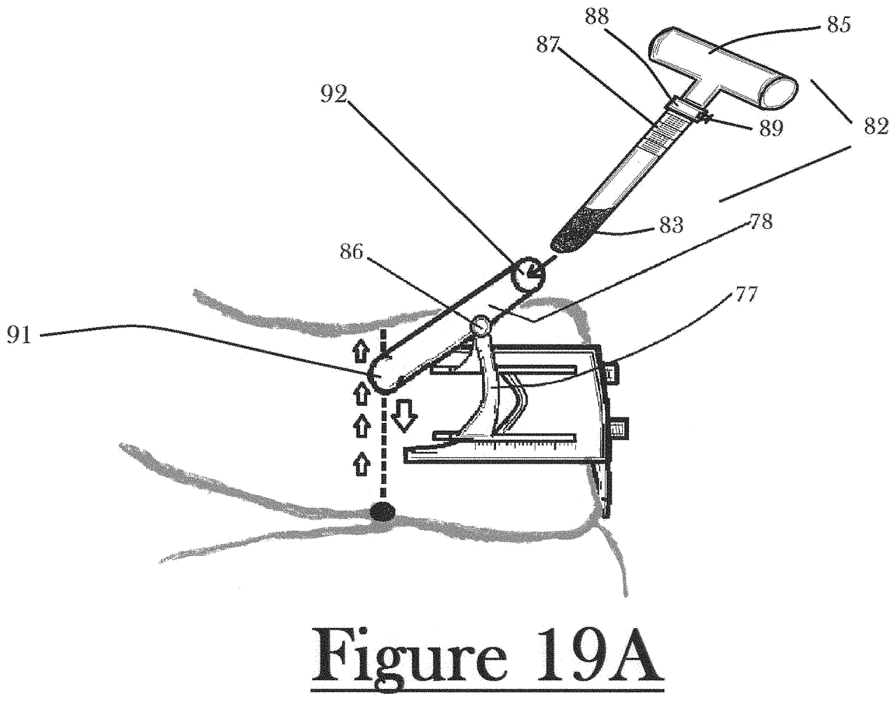

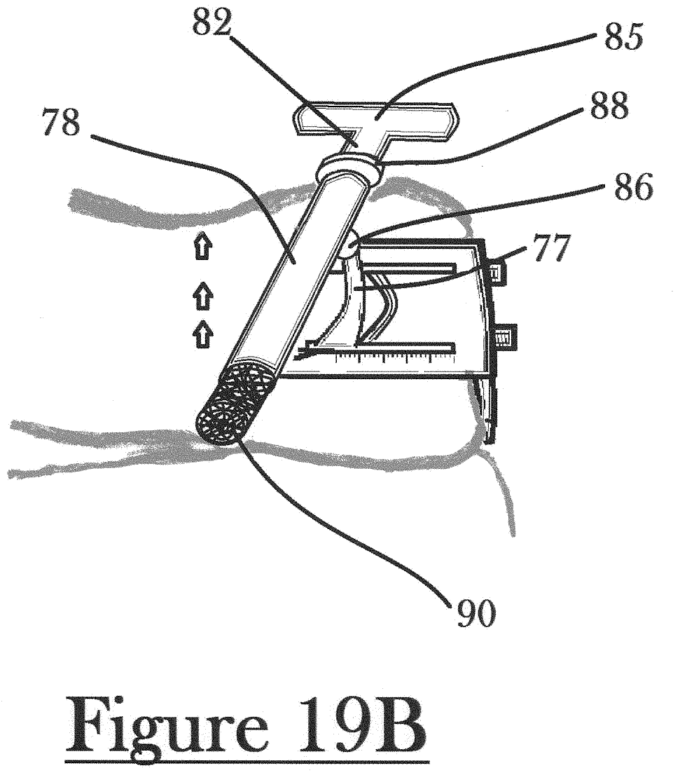

As has been stated on numerous occasions in this disclosure, a drill would be utilized in order to achieve the osteotomy. In one preferred embodiment, demonstrated in FIG. 18, the drill is a “T-shaped,” apparatus 82 with a leading end 83 of which is provided with multiple sharpened irregularities which are sharp enough to cut bone. The leadingmost edge 90 of the drill 83 is somewhat rounded so that it can penetrate bone most easily, creating the initial surgical corridor into the lamina; the irregular bone-cutting surface continues up the sides of the instrument creating a cylindrical cutting blade or “side cutting drill,” in addition to the front cutting drill; hence, after the caudalmost edge of the lamina is entered into the desired depth, the drill can be carried along the entire caudocranial course of the lamina with a single, smooth actuation. The central segment 84 of the instrument is then continuous with both the leading end 83 and the trailing actuating end 85. A portion of the central segment 84 is provided with calibrations 87, which in accordance with the data developed by the software program can determine the depth to which the drill 82 is to be carried so as to prevent inadvertent entry into the dural sac, or more ominously, mechanical injury to the spinal cord. This is achieved by providing a stop collar 88, which is positioned around the external diameter of the central segment 84. The stop collar is positionable with reference to the calibrations 87, and in this embodiment, is secured with a securing screw 89. Obviously, many other alternative embodiments of the mechanism to lock the stop collar in place are known to the technology and can be anticipated by those familiar with the art. For instance, threading could be provided to external surface of the trailing end of the central segment, with the stop collar being provided with complementary threading along its internal surface. Such a mechanism would again allow for precise adjustment of the depth to which the drilling can be carried. Once the drill has been carried through the bone to the level where the stop collar is brought against the trailing end 91 of the cradle 78, the drill cannot be carried any deeper; however, because of the side cutting feature, as the drill continues to spin and is precessed along the proposed osteotomy site, the osteotomy is achieved. In this example, the actuation end 85 is a simple “T handle,” but numerous embodiments, such as round handles, multi-arm handles, and many other such embodiments which are known to or acceptable to the art are all incorporated within the spirit and scope of this invention. Furthermore, those familiar with the art can anticipate a power-driven drill, either pneumatically driven, electrically driven, or any other type of power drill available to or acceptable to the art are also incorporated into the spirit and scope of the invention.

After slidably repositioning the saddle/cradle complex 77/78, the drill 82 can be disposed through the cradle 78 in preparation for achieving the osteotomy, as shown in FIG. 19A. It can be seen that the saddle 77 has been slidably repositioned so that the leading end 91 of the cradle 78 has been brought against the proposed site of the laminotomy (interrupted line); the leading end 83 of the drill 82 is passed through the central channel 92 of the cradle 78. Utilizing the swiveling mechanism 86, the complex is rotated caudally, as indicated by the single large open arrow; the stop collar 88 is adjusted to the calibrations 87 along the shaft, in accordance with the software program; the securing screw then sets the stop collar at the proposed optimal level. In FIG. 19B, the rotational mechanism 86 of the saddle 77 has positioned the cradle 78 such that the leadingmost end 90 of the drill 82 is actuated by rotating the trailing end 85, causing the leading end 90 to create an osteotomy at the caudal most position on the bone. By redirecting the drill 82 and cradle 78 complex cranially, as indicated by the series of smaller open arrows, the osteotomy completed is completed along the proposed course, indicated by the interrupted line. They stop collar 88 [presumably] prevents the drill from carrying the osteotomy completely through the bone and injuring delicate tissues which are “deep” to the bone, such as the spinal cord. It is anticipated that the osteotomy will allow a very thin shell of bone to be retained which is removed by a standard rongeur type instrument, which can also be used to remove the yellow ligament, exposing the dura.

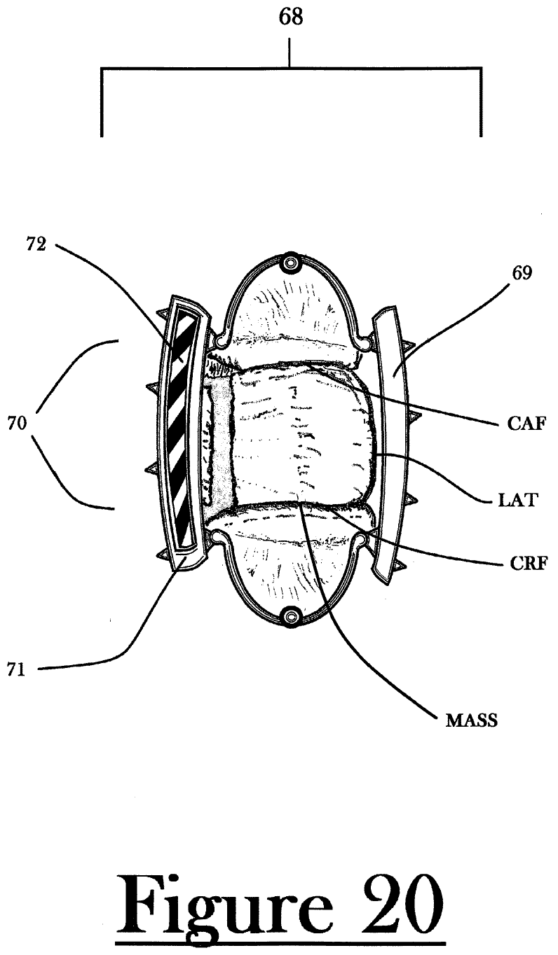

FIG. 20 shows the completed left sided osteotomy as seen through a top view, which would be essentially a surgeon's view. Again, the retractor 68 with the complex medial blade 70 with the frame 71 and the movable blade 72, as well as the lateral blade 69, provides visual access to the lateral mass MASS, as well as the cranial CRF and caudal CAF facet joints (the position of these are important when performing a multilevel procedure). The lateral edge LAT of the lateral mass is also seen, which is important when placing the lateral anchor. The osteotomy OSTEO is the discontinuity in the bone which is illustrated in gray. The reader is invited to compare this to FIG. 13, where the proposed site of osteotomy is represented by a series of solid ovals.

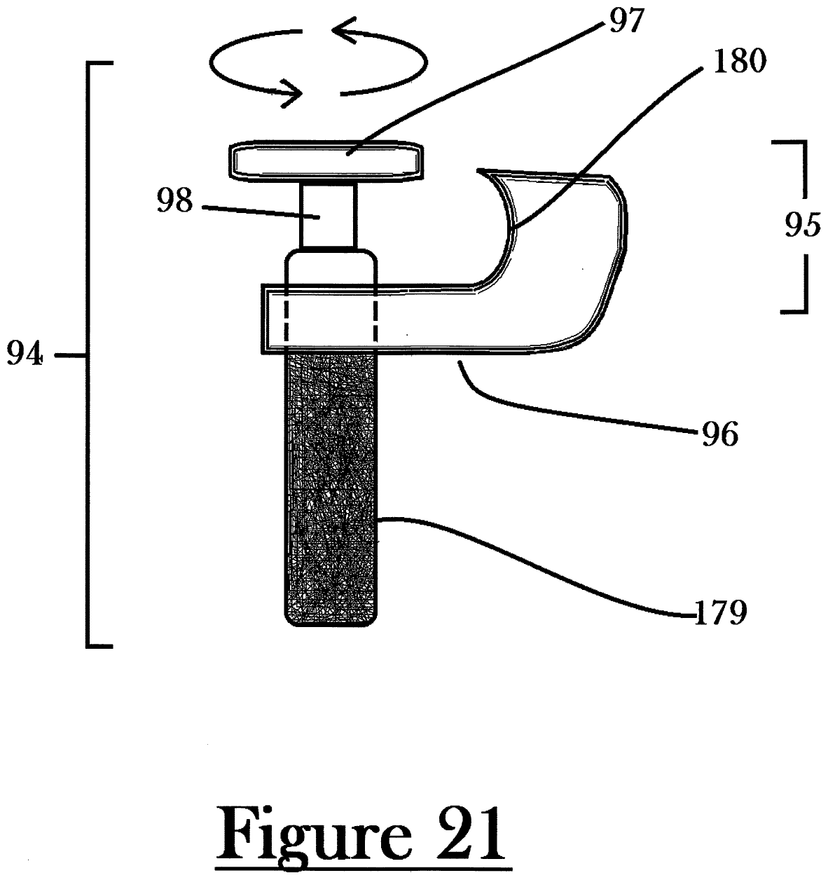

The operative field is now prepared for the placement of the lateral mass anchors. This is achieved by first preparing the medial aspect of the lateral mass to accept the medial element of the lateral mass anchor such that this portion of the lateral anchor does not impart any mass effect into the lateral aspect of the spinal canal as seen on the transaxial view. This can be accomplished by any device the surgeon nominates, using a “freehand,” technique; alternatively, this may be achieved with the use of a uniques device which shall hereinafter be known as the medial osteotome 94. This is shown in a lateral perspective in FIG. 21, where is can be seen that the instrument 94 is comprised of a lateral vertical segment 95 which serves as a handle to be used by the surgeon's nondominant hand, with a finger rest 180 to provide lateral traction so that the osteotomy is carried laterally into the medial aspect of the lateral mass. A rotatable handle 97 transmits the actuation, as indicated by the arrows, to the shaft 98 and ultimately to the leading end 179, which is a drill and will create a limited bony defect in the medial aspect of the lateral mass, into which the medial element of the lateral anchor is insinuated.

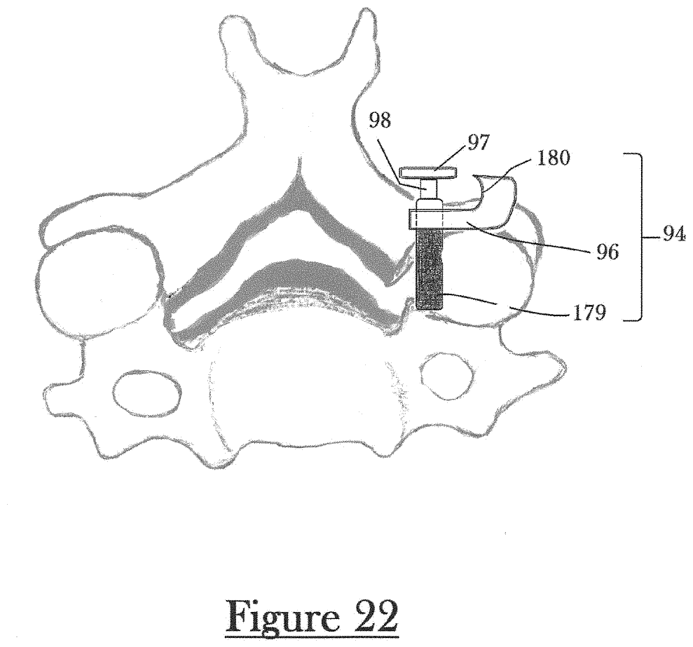

The use of this instrument is illustrated further in FIG. 22, a transaxial view of the medial osteotome 94 is being utilized against the left lateral mass of an exemplary vertebra. The leading end 179 is a cylindrical, side-cutting drill which is dimensioned so as to create a partially decorticated tract in the medial aspect of the lateral mass into which the medial element of the lateral anchor will be positioned. The dural aspect of this element is provided with a camber-like surface to deflect the dura away and prevent the dura from being engaged within the rotating drill. This defect is ideally created so that the central aspect of this defect is completely decorticated whilst the lateral aspects of the defect retain some cortical bone that the lateral aspects of the medial element can be securely positioned against. The leading end 179 of the instrument 94 is actuated by rotation of a handle 97 which has been provided to the trailing end (implied by the circular arrows), this rotation being transmitted through shaft 98 to the leading end. The surgeon's (presumably non-dominant) hand stabilizes the instrument 94 with a finger rest 180 by which the surgeon can utilize gentle lateral traction while actuating the drill 179 so as to develop the defect deep to the cortical surface of the medial aspect of the lateral mass.

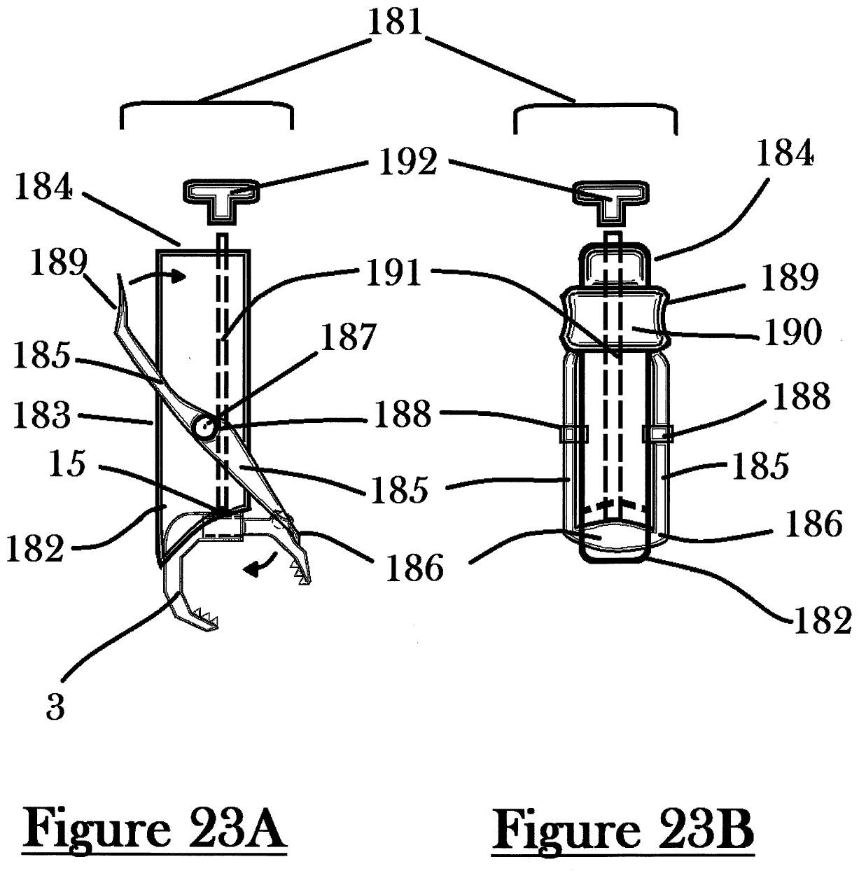

With the lateral masses now fully prepared to receive the lateral anchors, an apparatus for implanting the anchors is introduced. FIG. 23A is a lateral view of a preferred embodiment of such an apparatus 181, with a lateral anchor 3 positioned between the leading end 182 of this apparatus 181 and the leading end 186 of actuating arm 185. This arm 185 is secured at pivot point 187 to the central segment 183 of the apparatus 181 by pivot axle 188, which creates a [mediolateral] plane of movement of the actuating arm 185 when positioned within the surgical field. Actuating plate 189 of arm 185 is found at the trailing end 184 of the apparatus 101 and is provided with a broad central area 190 (best seen on frontal view 23B) which serves a thumb/finger actuating panel. By digital pressure on the panel 190, the leading end 186 is compelled towards the trailing end 182 of the apparatus; when a lateral mass anchor 3 is in position against the target bony areas, the elements of the anchor 3 are compressed towards each other, thus compressing the anchor 3 against the lateral mass. The apparatus 181 is also provided with an internal screwdriver 191 (denoted by interrupted lines along its internal course through the apparatus) to which a detachable handle 192 can be reversibly coupled so that the screwdriver can be actuated, locking the lateral anchor securing screw 15 and hence locking the anchor 3 in the desired position.

Certain features of this apparatus 181 are best appreciated in a frontal view, illustrated in FIG. 23B. Perhaps the most important feature is that the actuating arms 185 are located bilaterally along the exterior of the apparatus 181, with the leading end 186 and the actuating plate 189 continuous and monolithic with the arms 185 such that the entire structure, in the preferred embodiment, is monolithic and related to the circumferential exterior of the apparatus 181. Such a design will provide superior leverage when compressing the anchor 3 into position (for simplicity, the anchor is not demonstrated in this projection). It can also be noted that the apparatus 181 is not cylindrical but rather wider along the mediolateral plane (as positioned within the operative field) and more narrow in the orthogonal plane, in a fashion similar to the lateral anchor itself. This again is felt to increase the efficiency of the apparatus 181. In this projection, one sees the the broad central area 190 of the actuating panel 189 of the actuating arms 185, and can appreciate, that owing to the rotation around the pivot axles 188 (located bilaterally/FIG. 23B), digital pressure on the central panel 190 in the direction of the upper curved arrow would compel the leading end 186 actuating arms 185 to be be repositioned in the opposite direction as indicated by the lower curved arrow in FIG. 23A. This would in turn compress the elements of the anchor 3 towards each other and will result in compression of the anchor 3 against the lateral mass.

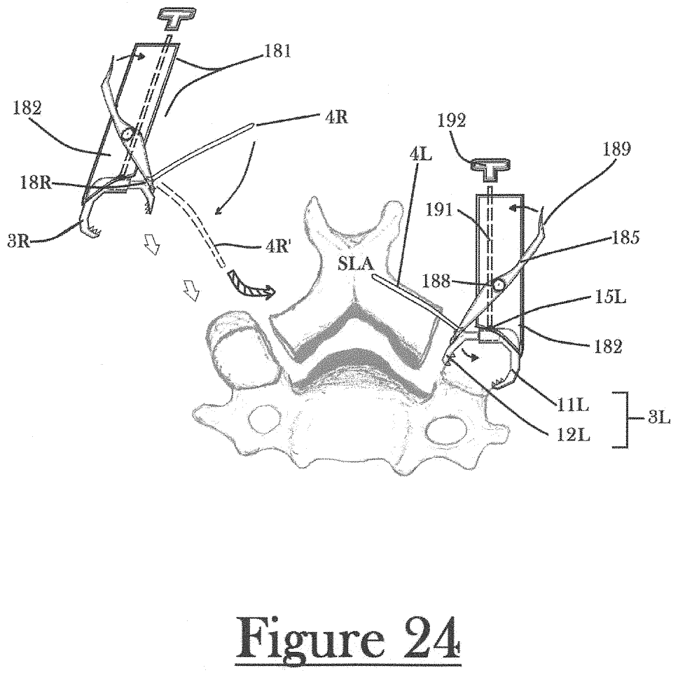

The implantation of the preferred embodiment of the lateral anchors includes negotiating the elevating stabilizers 4R, L into position. This process is illuminated in FIG. 24, wherein a cranial transaxial view of lateral anchors being secured onto an exemplary cervical vertebra. On the viewer's left, the right-sided anchor 3R is mounted onto the leading end 182 of the implantation apparatus 181 and is being brought into the operative field. The trailing end 18R of the elevating stabilizer 4R is irreversibly coupled to the anchor 3R, so implantation requires manipulation of the stabilizer 4R. It is rotated around its trailing end 18R, as indicated by the solid arrow, with its “ghosted,” image being introduced into the surgical field initially as represented by the interrupted outline of the stabilizer 4R′. Then, following the course indicated by the stippled arrow, it is insinuated between the muscle fibers still attached to the spinolaminar arch SLA (fibers not shown), so it can be reversibly coupled to the spinous anchor (also not illustrated in this image). The anchor 3R is brought into position so as to be deployed against the lateral mass, as indicated by the open arrows. The final position of the anchor 3L and stabilizer 4L are seen on the right side of the Figure. Once so positioned, as previously described, the surgeon applies digital pressure (arrow) to the trailing end 189 of the actuating arms 185 which are rotated around the pivot axle 188. The leading end 182 of the apparatus 181 positions the lateral element 11L of the anchor 3L firmly against the lateral aspect of the lateral mass, so that with rotation of the actuating arms 185, the medial element 12L is repositioned securely against the medial aspect of the lateral mass, as indicated by the arrow. Once the screwdriver 191 is actuated by the handle 192, the securing screw 15L locks the lateral anchor 3 locks in position, providing excellent fixation against the lateral mass.

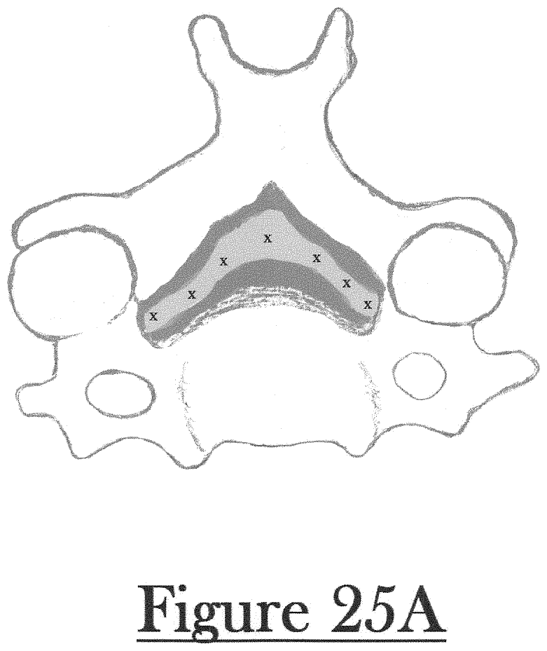

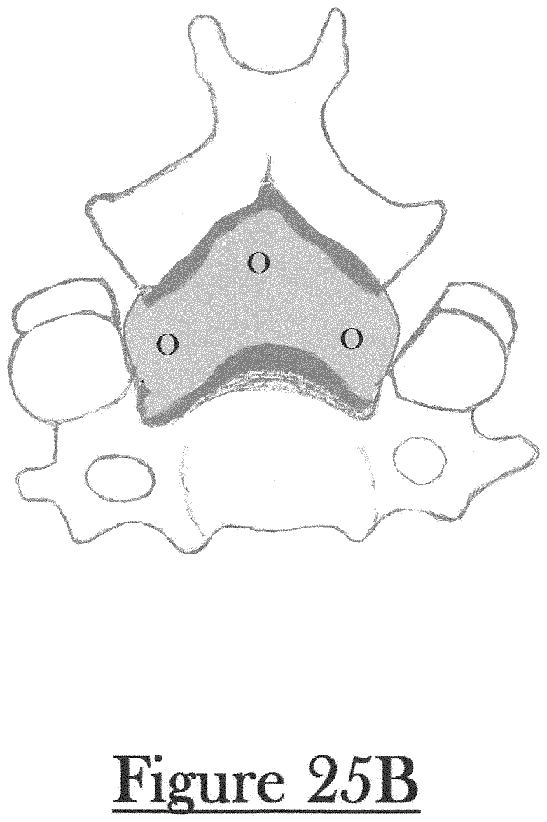

The software program will also provide the surgeon with an opportunity to determine how much the dimensions of the spinal canal would be augmented. In some situations, in which moderate but very symptomatic stenosis is present, only a modest degree of augmentation is necessary; in cases of severe stenosis, a greater degree of augmentation must be achieved in order to maximize the chances of improvement. An example of severe stenosis is seen in FIG. 25A, in which the light gray areas demarcated by the “x” markers represents the actual dimensions of the canal. This would be maximally decompressed by the proposed reconstruction in FIG. 25B, wherein the canal dimensions have been reconstituted as demarcated by the “o” markers. The software program would dictate how much elevation of the spinolaminar arch is required to achieve this result.

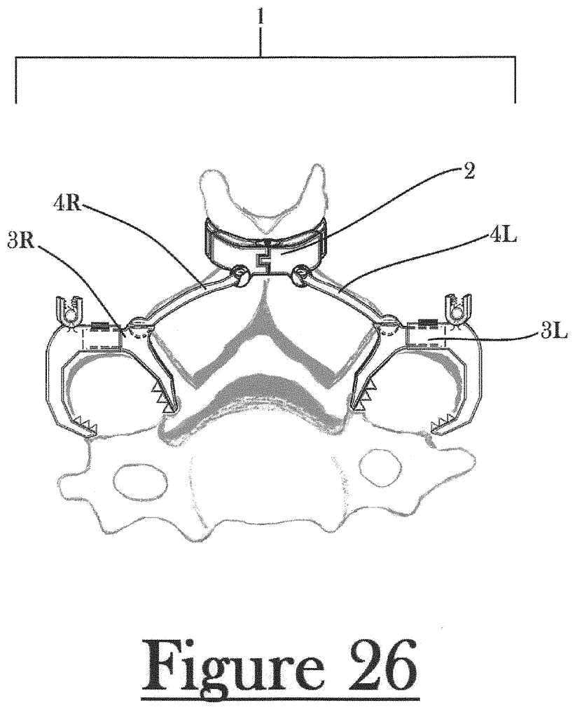

Executing this augmentation after securing the CMIL 1 into position will result in a predictable and reproducible expansion of the spinal canal. In FIG. 26, a cranial transaxial view of an exemplary vertebra is seen with the CMIL 1 represented by the spinous anchor 2 coupled to the left 3L and right 3R lateral anchors by the left 4L and right 4R elevating stabilizers. The pathology is still noted as the canal augmentation has not yet been accomplished.

There are any number of options available to the surgeon in order to accomplish elevation of the spinolaminar arch. Clearly, the first of these options would be to utilize a device to temporarily grasp the bone at one or more sites of the spinolaminar arch and elevate the arch using this form of leverage, as opposed to elevating the spinolaminar arch by leveraging against the hardware which is secured against it, namely the spinous anchor directly, or leveraging against the elevating stabilizers which are, in turn, coupled with the spinous anchor and in that fashion elevating the spinolaminar arch.

Logically, it would seem that in the preferred embodiment, the arch should be elevated by temporarily grasping one or more bony surfaces of the arch and elevating it. The primary concern about leveraging against the instrumentation is concern that the instrumentation could be loosened or even dislodged during this maneuver.

Certainly, one option which many surgeons would be comfortable with would be to simply grasp the spinous process with surgical forceps and elevate the arch “freehand.” This may well result in an acceptable surgical outcome but would not provide a guarantee of augmenting the spinal canal in a manner which was predicted preoperatively. Therefore, if the instrumentation is made available to them, other surgeons would likely choose a more precise and predictable technique for achieving canal augmentation.

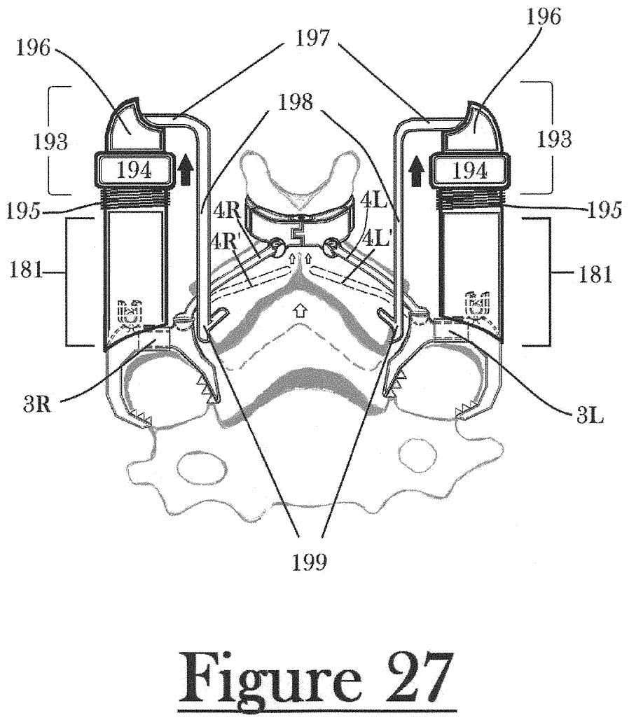

Hence, in the preferred embodiment, the arch is leveraged into the desired position by a system demonstrated in FIG. 27. In this transaxial view, this system utilizes caps 193 which are reversibly coupled to the trailing ends of the apparati 181 which implant the lateral anchors 3. These caps 193 are provided with a rotatable displacement ring 194 which is advanced along threading 195. As these are advanced, positioning tips 196 are in turn repositioned posteriorly (away from the spine). These tips 196 are in turn continuous and monolithic with elevating links 197 which are continuous with elevating shafts 198, which are in turn continuous and monolithic with securing claws 199. The securing claws 199 are brought against the cut edges of the spinolaminar arch which have been rendered as free edges by the lateral osteotomies. By correlating the displacement of the spinolaminar arches with the amount of rotation of the displacement rings 194 (solid arrows), the surgeon can precisely predict the amount of spinal augmentation which occurs with a measured amount of rotation of the displacement rings 194 with consequent displacement of the securing claws 199. This is indicated by the interrupted line indicating the original configuration of the spinal canal as well as the large open arrow indicating the direction of displacement of the spinolaminar arch. In a corresponding fashion, the original position of the elevating stabilizers 4R′, L′ is indicated again by interrupted lines “ghosting” their configuration; they are displaced in the direction indicated by the small open arrows, and their final position is indicated as 4R, L. Logically, elevating the spinolaminar arch by lifting up its lateral edges may again have some advantages over pulling the arch up by the spinous process.

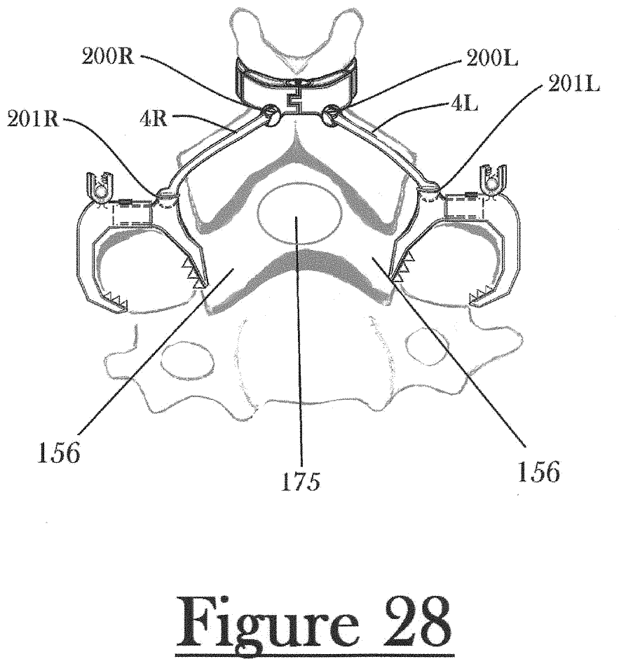

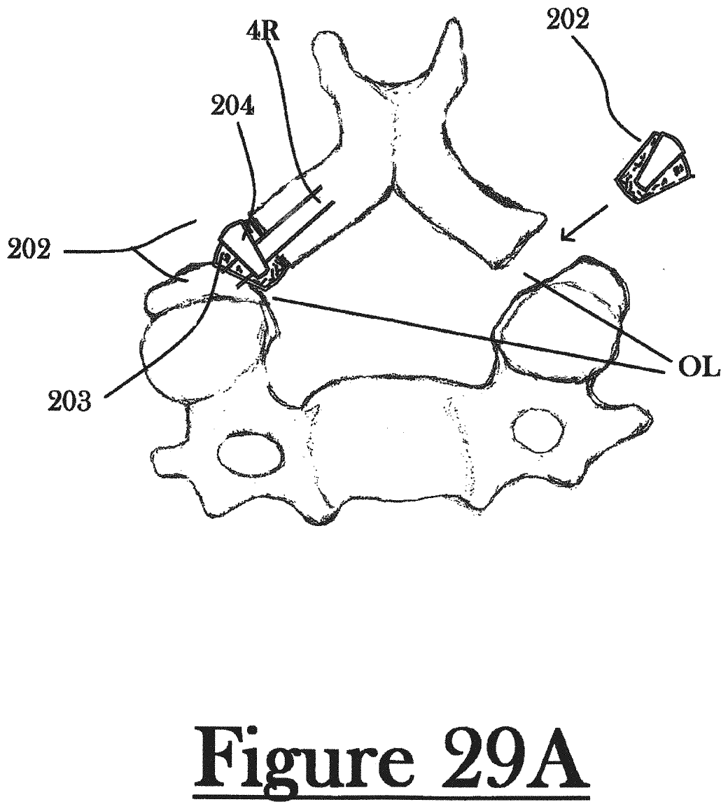

A final result of the spinal augmentation can be seen in the transaxial view offered in FIG. 28. Here, it can be seen that the spinal canal 156 has been widely decompressed by the augmentation of the canal consequent to the elevation of the spinolaminar arch. The spinal cord itself 175 has re-assumed its normal [ovoid] configuration, with cerebrospinal fluid circulating around it within the canal. After the final position of the CMIL is achieved, the elevating stabilizers 4R, L are locked in position by securing their leading ends with the locking screws 200 of the housing mechanism of the spinous anchor, as well as locking the securing rings 201 which secure their trailing ends.

Many surgeons would feel that the lateral osteotomies are relatively narrow and would not require any type of bone graft substrate, particularly given the continued blood flow to the spinolaminar arch. However, others would suggest that the purpose of the laminoplasty is to maintain a posterior cervical arch, and that this is best achieved by promoting a fusion between the arch and the medial aspects of the lateral masses. This can be achieved by a number of techniques but included in this application is the description of a cadaver graft which is pre-fitted in accordance with the size of the lateral osteotomy, and which when manufactured is provided with an attachment brace so that it can be secured to the elevating stabilizer and thereby held in place.

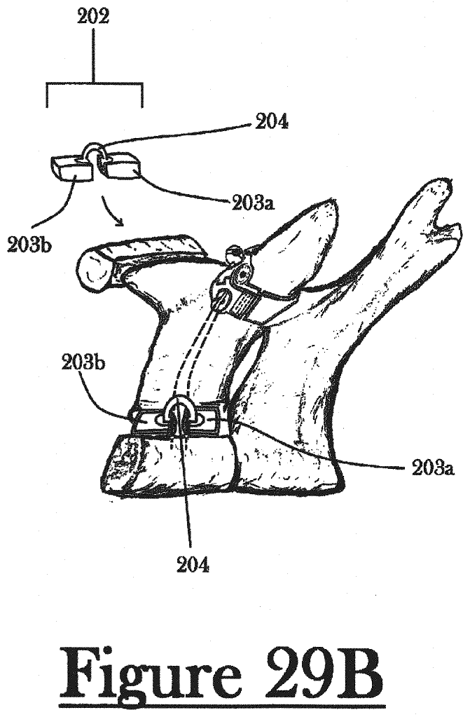

Examples of such a graft/brace implants 202 are shown in FIGS. 29A/B. In the first Figure, which is a transaxial view demonstrating the lateral osteotomies OL. On the viewer's right, the implant 202 is seen on its course (arrow) to fill the osteotomy OL. On the viewer's left, the implant 202 is seen filling the defect created by the osteotomy OL. In order to better illustrate the critical structures, the entire CMIL is not shown, although it is to be remembered that at this point in the procedure the entire construct would be in place. Again for illustrative purposes, a portion of the elevating stabilizer 4R is shown with the brace 204, which in this drawing assumes a more “hook” like appearance, being disposed over the top of the stabilizer 4 which it is then pressure fitted against. The graft portion 203 of the implant 202 is seen caudally to the brace 204; however, graft components can be positioned on both sides of the hook-like brace 204. Various techniques to secure the brace 203 to the stabilizer 4R can be anticipated, including a securing screw, “cold welding,” rotatable nuts, and others. Any and all such techniques would be included within the spirit and scope of the invention.

FIG. 29B is a left sided elevational view of a motion segment in which the superior/cranial vertebra is undergoing canal augmentation utilizing the CMIL. The spinous anchor is in place, but again for illustrative purposes, the lateral anchors have been removed. The implants 202 are slightly modified inasmuch that the brace 204 in this drawing is represented by an arched bridge-like component interpositioned between two implants 203a, b. The brace 204 is again pressured fitted against the stabilizer 4 (shown in relief), as seen in the foreground.

Cervical stenosis typically involves 2 or more levels, and the average number of levels of decompressive laminectomy is 2.5. Therefore, a multilevel construct must be disclosed. FIG. 30A shows CMIL systems having been applied to two consecutive cervical levels. Cradles 31L, R have been provided to the lateral anchors 3L, R of the cranial vertebra, while cradles 31L′, R′ have been provided to the anchors 3L′, R′ of the caudal vertebra. When a multilevel lateral fusion is performed, many surgeons would want to avoid use of the grafts to fill the lateral osteotomies so as to not to promote recurrent stenosis, and for that reason have not been included in this illustration. The connecting rods 205L, R are then secured into the cradles 31L, R; 31L′, 31R′ by locking screws 206L, R; 206L′, R. Of course, any number of levels can be incorporated into the construct by simply utilizing a longer rod.

Therefore, while a number of surgeons would be satisfied with the construct illustrated in FIG. 30A, many others would insist on placement of bone graft substrate to fill the lateral osteotomy sites, which is depicted in FIG. 30B.

Stabilizing one or more motion segments generally implies that fusion also be performed at the same levels. This is considered universally as a mandate when discussing the lumbar and lower thoracic spine; however, it is somewhat more controversial in the cervical spine. There are several well respected authorities who regularly advocate that merely stabilizing the cervical facet joints will result in autofusion, but the clinical evidence of this is somewhat unclear. Therefore, a final option offered to the surgeon is a unique pre-fabricated cadaveric graft which is configured and utilized to promote maturation of a fusion mass along the lateral aspect of the column of lateral masses; this is often referred to, in surgical parlance, as the “lateral gutter.” An exemplary illustration of such a graft is seen in FIG. 31, where the two [isolated] lateral anchors 3, 3′ are seen in a frontal elevational view. For illustrative purposes, the target bony structures have been omitted from this image. Threaded apertures 25, 25′ have been provided to the lateral vertical segments of the anchors which receive screws 213, 213′ which are, in turn components of cradles 211, 211′ found at each end of the implant 210. Securing the implant 210 to the lateral aspects of the anchors brings the cadaveric graft component 212 (thin double lines) of the implant 210 against the bony surfaces of the lateral masses, thus promoting fusion; many surgeons would decorticate this bone to further promote maturation of the graft substrate 210 into fusion mass. In an alternative embodiment, the cradles 211, 211′ are slidably coupled with the graft component 212, thus reconciling differences in the distance between anchors 3. 3′ from level to level. Other alternative embodiments can be conceived and anticipated, in particular by those familiar with the art; all such embodiments are within the spirit and scope of this invention.

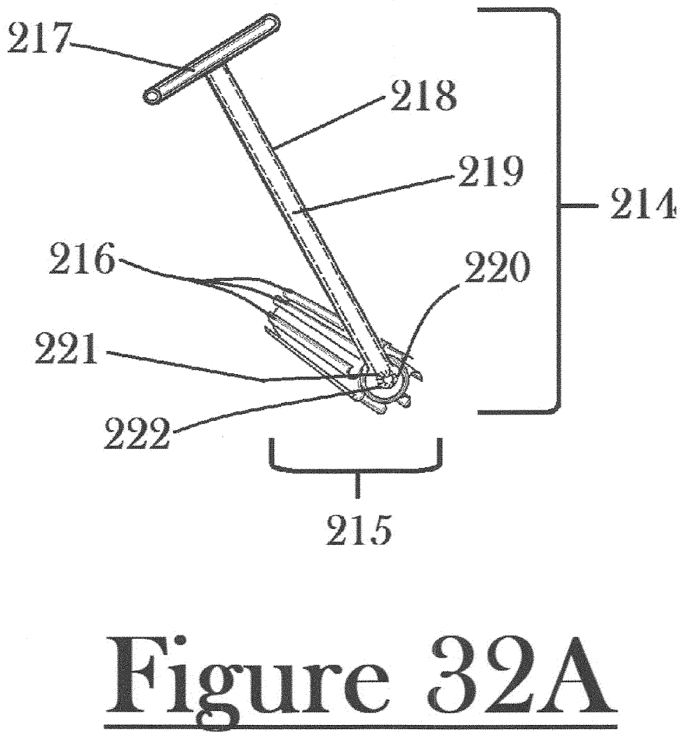

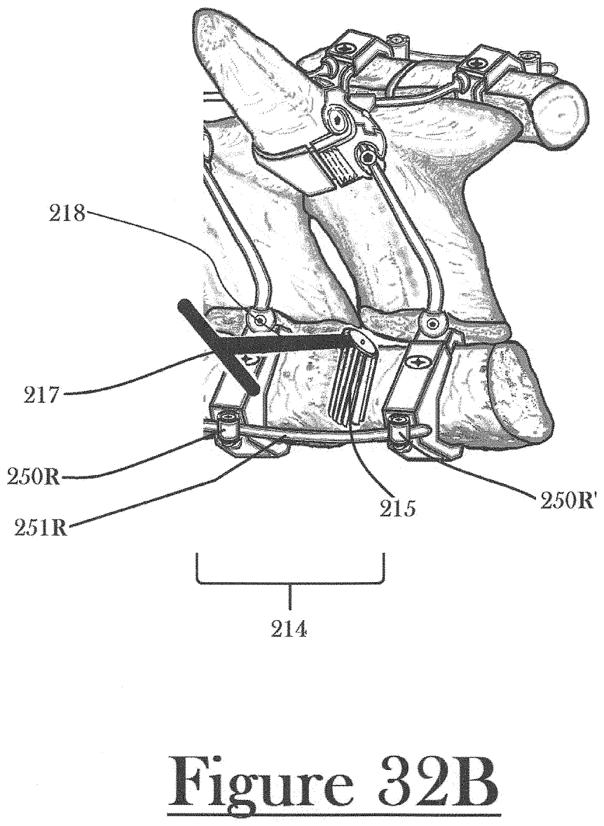

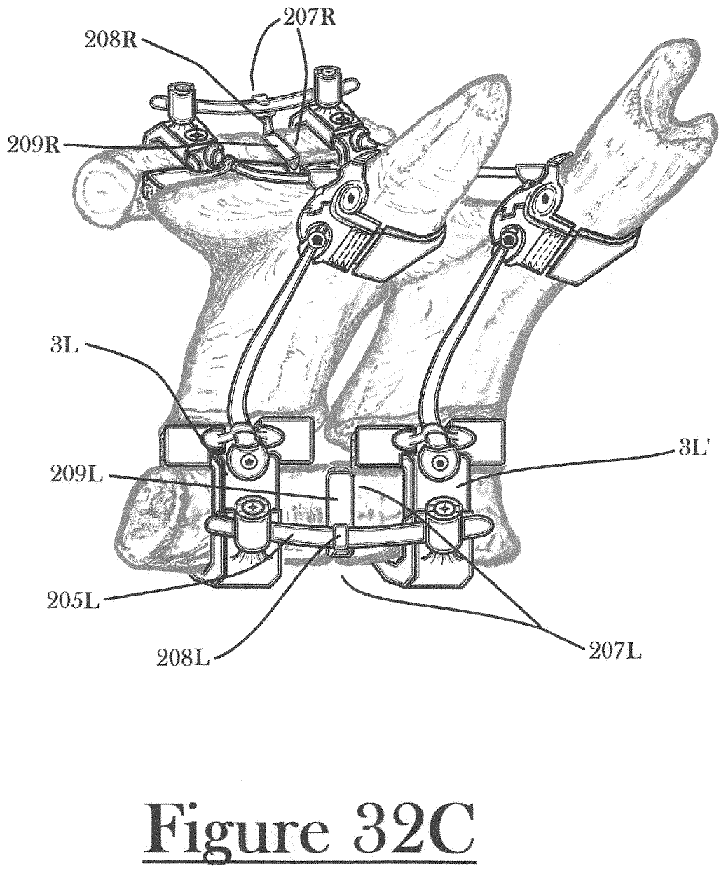

It is the opinion of many surgeons that a posterior cervical fusion would include fusion of the facet joints. This is often simply achieved by removing the joint cartilage and decorticating the adjacent bony surfaces. However, evidence suggesting that a significantly higher rate of fusion occurs with the use of graft material. A device known as the facet extractor 214, pictured in FIG. 32A, extirpates the facet joints while creating a perfectly dimensioned cavity which receives a prefabricated cadaver graft. In the preferred embodiment, a cylindrical drill 215 is provided with a series of curved blades 216 extending from the surface. This component 215 is irreversibly coupled with a shaft 218, the trailing end of which is a handle 217 that rotates an internal drive shaft 219. This, in turn, actuates a beveled gear 221 within the gear housing 220 located at the coupling point, this actuation rotating the gear 222 of the cylindrical drill 215. An alternative embodiment (not pictured) utilizes a box chisel configured to be impacted along the orientation of the joint. Other alternative embodiments can be conceived and anticipated, in particular by those familiar with the art; all such embodiments are within the spirit and scope of this invention.

FIG. 32B demonstrates the Facet Extirpator 214 in use on a right-sided facet joint. The cylindrical drill 215, actuated by the handle 217, is driven into the facet joint along the angle of the joint. A variety of options including a premeasured mark on the shaft 218, as well as a stop configuration placed on the shaft (not pictured here) directs the depth to which the drill is carried in order to core out the ideal cavity. Depicted herein is an embodiment in which the cradles 250R, 250R′ project laterally into the lateral gutters from the lateral mass anchors 3R, 3R′, with the connecting rod 251 extending through the lateral gutter. This represents an extremely “low profile,” construct, which is considered most desirable.

The resulting configuration is illustrated in FIG. 32C, wherein it can be seen that a unique [left-sided] Facet implants 207(L,R) have been interposed within the bed of the drilled-out facet joints. The implant 207 is comprised of a cadaveric bone portion 209 which is prefabricated and is mounted on a metallic or plastic brace component 208 which is configured so that it can be pressure-fitted onto the connecting rod 205; the left-sided construct is more completely illustrated, where it can be seen that the brace component 208L of the implant 207L is fitted onto the rod 205L which couples the lateral anchors 3L, 3L′. The coupling to the connecting rod stabilizes the position of the facet graft and maintains it in place. As has been used throughout this specification, the suffixes L, R are used to designate the left or right sided implants.

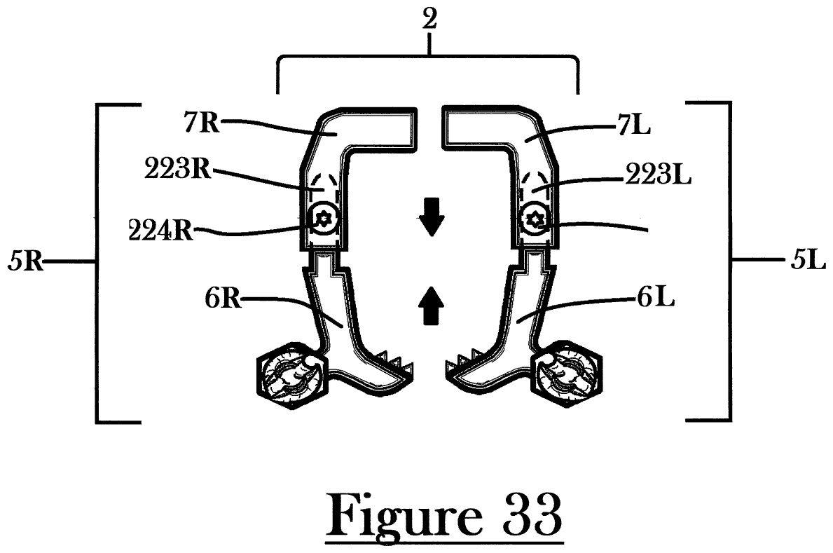

For instance, in the preferred embodiment disclosed above, the cranial and caudal elements are coupled be a ratcheting mechanism. Those familiar with the art can envision a number of mechanisms that could achieve such coupling; one such mechanism is illustrated in FIG. 33, which is a top view of the anchor 2 showing that the right 5R and left 5L members are independent, which is in of itself a unique embodiment. Furthermore, the elements on each side 6R, 7R and 6L, 7L are compressed towards each other, as indicated by the solid arrows, with the repositioning governed by slidably repositionable coupling extensions 223R, 223L which are locked in place by securing screws 224R, 224L.

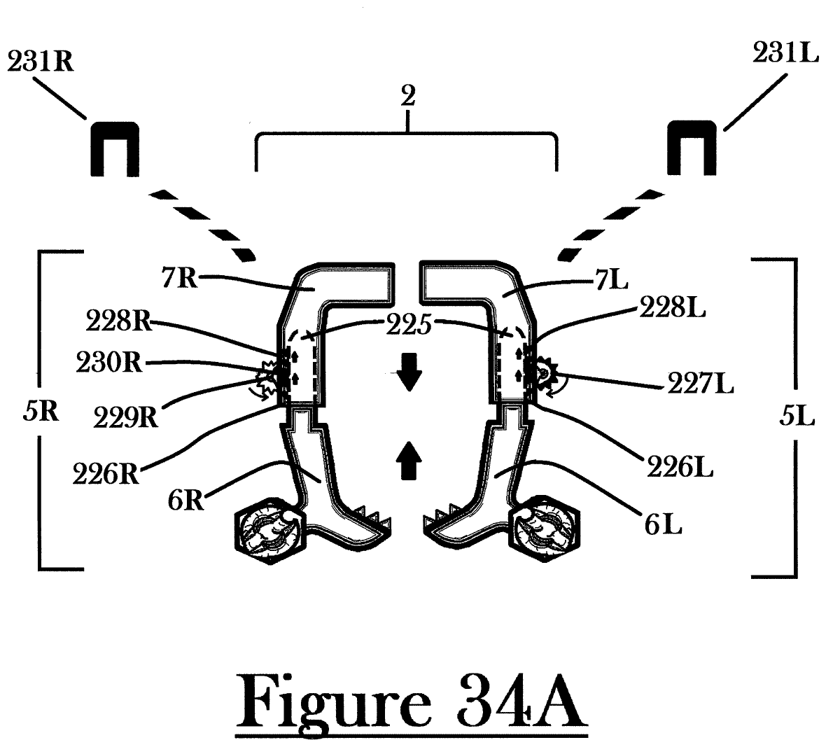

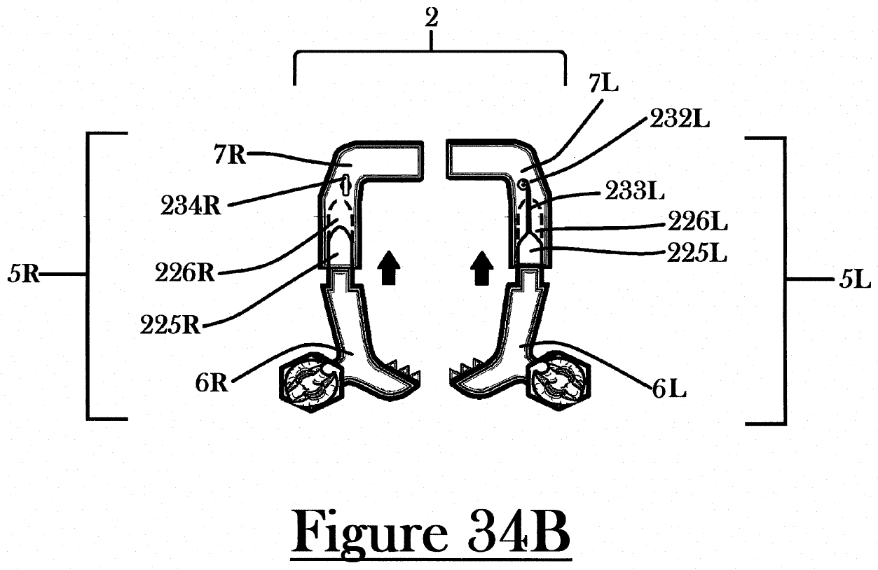

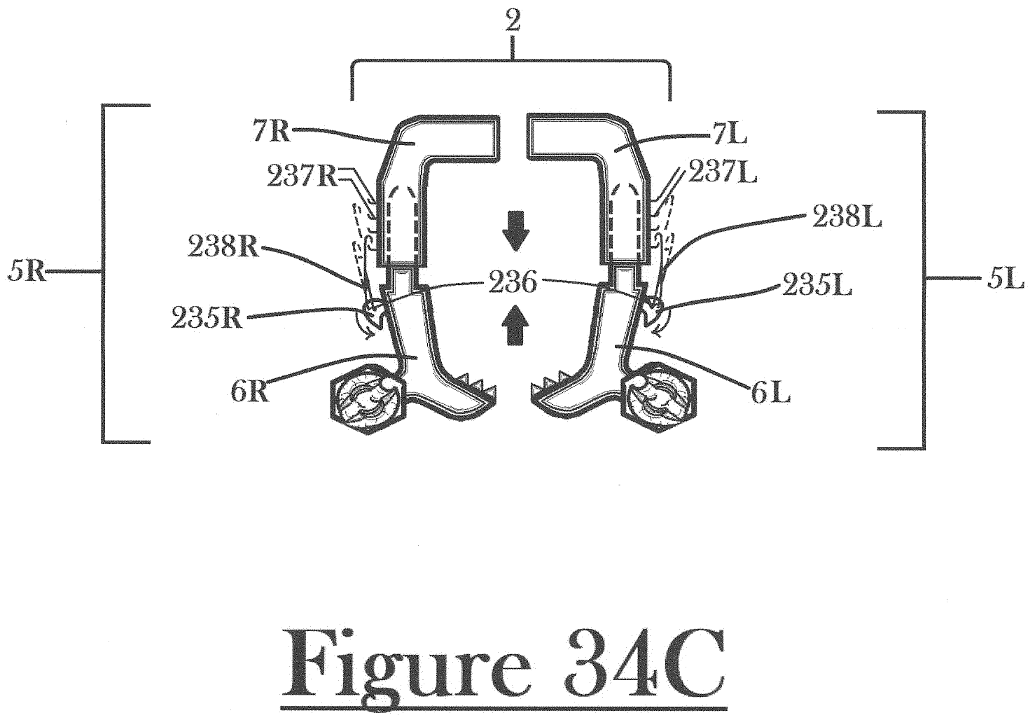

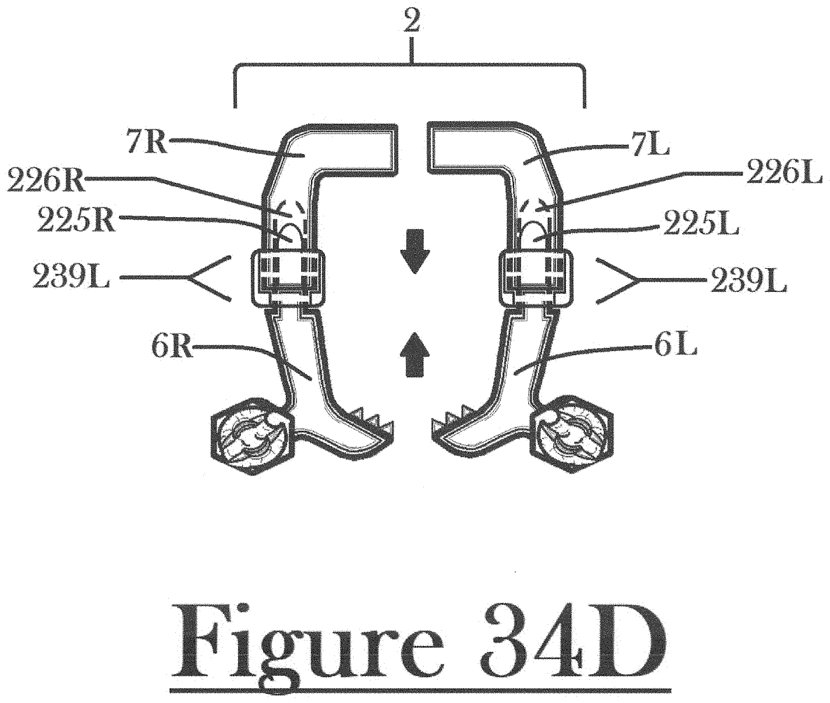

Other mechanisms which accomplish securing the spinous anchor in position are included in the top views seen in FIGS. 34 A/B/C/D. In the first of these, a repositioning gear 227 is provided to the leading end of the lateral aspect of the caudal element 7. To illustrate this with the greatest clarity, the gear 227 is accentuated on the left 227L; conversely, the right side shows with greater clarity the axle 229R of the gear (not enumerated on the right side, but presumed to be 227R) which is supported by gear extension 230R. This configuration permits the axle 227 to be rotated, as indicated by the curved arrow, engaging teeth 228 (R, L) which have been provided to the lateral aspects of coupling extensions 225 (R, L). By engaging the teeth 228, the gear 227 advances the coupling extensions 225 of the cranial elements 6 into the coupling chambers 226. This actuation, of course, compels the two elements 6, 7 toward each other, securing the anchor 2 against the spinous process. Once the surgeon is satisfied with the positioning of the anchor, the gear 227 can be locked by a variety of mechanisms; one such mechanism is shown herein, whereby a locking brace 231 R. L can be secured against the gear, as indicated by the curved interrupted lines. This would, of course, lock the entire anchor configuration in place.

FIG. 34B reveals another embodiment by which the elements of the member can be approximated. In this configuration, a repositioning axle 232 is provided to the coupling chamber 226 and configured such that its actuation places traction on a repositioning cable 233, which is irreversibly coupled with the coupling extension 225 of the cranial element 6. The internal configuration is seen best on the left L side of the anchor 2; best seen on the right R side is the repositioning handle 234R which actuates the system, and which is exterior to the element 7 and accessible to an actuating instrument. Actuation compels the cranial elements 6R, L toward the caudal elements 7R, L, as indicated by the solid arrows. This repositioning is shown by the solid outlines of the coupling extensions 225R, L and the outlines of the coupling chambers 226R, L, shown with interrupted lines, which will receive the extensions 225R, L. It is presumed that one of a variety of mechanisms which secure and lock the axle would also be incorporated into the design.

Additional variations in the mechanism governing the repositioning and locking of the spinous anchor can be seen in FIGS. 34C and D. In the first of these, coupling of the members is achieved with a bucket handle design. Specifically, the lateral aspect of the cranial elements 6R, L are provided with unique, useful, novel and nonobvious hemicircular element 235 which rotates around a fulcrum 236. The coupling with the fulcrums conveys the hemicircular elements rotation through approximately 180° as indicated by the semicircular arrow, and furthermore, conveys resistance near the extreme of the range of movement. In a corresponding fashion, the lateral aspects of the caudal elements 7R, L are provided with a series of projections 237 which are configured to reversibly couple with an extension 238 of the hemicircular element 235. The potential positions of the extension 238 are shown in relief. After the members 5R, L have been positioned, the extension 238 is brought against the appropriate projection 237, and the hemicircular element 235, which is portrayed in the initial, non-deployed position, is rotated through the hemi-arc. At the point, the hemicircular element 235 is rotated into the locked position, as indicated by the curved arrow, locking the anchor 2 in place.

FIG. 34D is an embodiment in which the cranial 6R, L and caudal 7R, L elements are coupled by the use of a cold weld. Specifically, the coupling extensions 225R, L again are disposed into the coupling chambers 226R, L, as in previous embodiments. However, in this alternative, a coupling collar 239R, L is provided to the junction of the two elements 6, 7 and upon satisfactory positioning of the anchor 2, the collar is deployed.

Many other mechanisms can be invoked including a piston screw mechanism, a spring-loaded mechanism, a geometric configuration locking mechanism, and certainly many others.

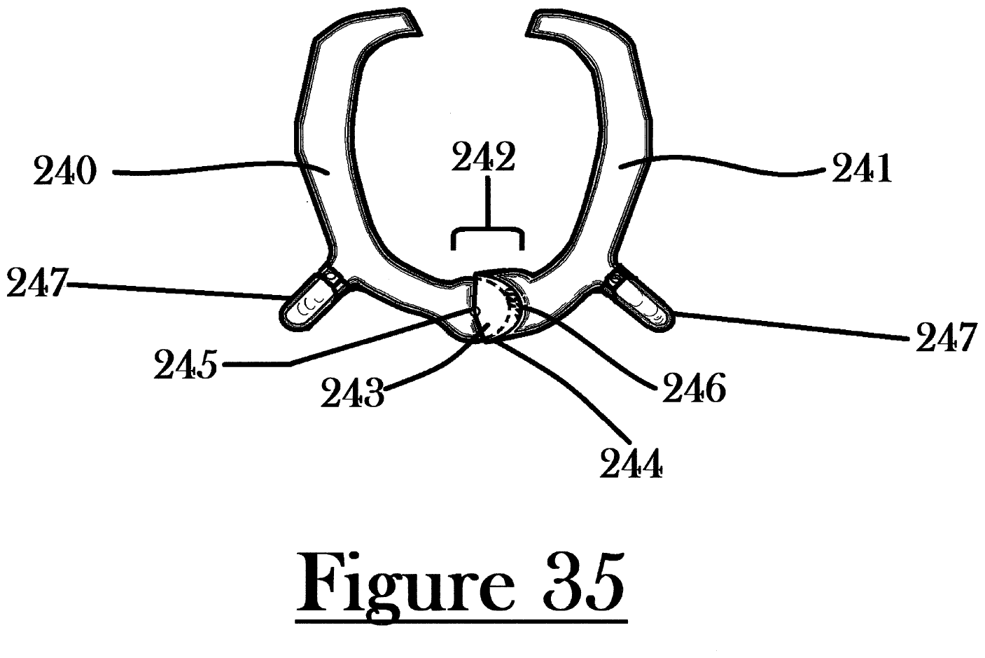

It is conceivable that the anchor could be comprised of members which are monolithic. An exemplary embodiment is portrayed in FIG. 35, which is a top view showing the monolithic right 240 and left 241 members, which are irreversibly but repositionably coupled at the cranial junction 242. The junction is a ball and socket joint, with the leading end of the right sided member 240 having the configuration of the ball 243 which fits into the socket 244 representing the leading end of the left sided member 241; the positions of the ball and socket could be reversed with respect to the members. It is anticipated that the embodiment would be splayed at the time of introduction, and the members 240, 241 are rotated around an axle 245 provided to the ball and socket joint 242, and approach each other until they are secured against the sides of the spinous process. A ratcheting mechanism 246 is provided to the interface of the ball and socket which locks the construct in place. Extensions 247 are provided to each member 240, 241 to couple with the elevating stabilizers.

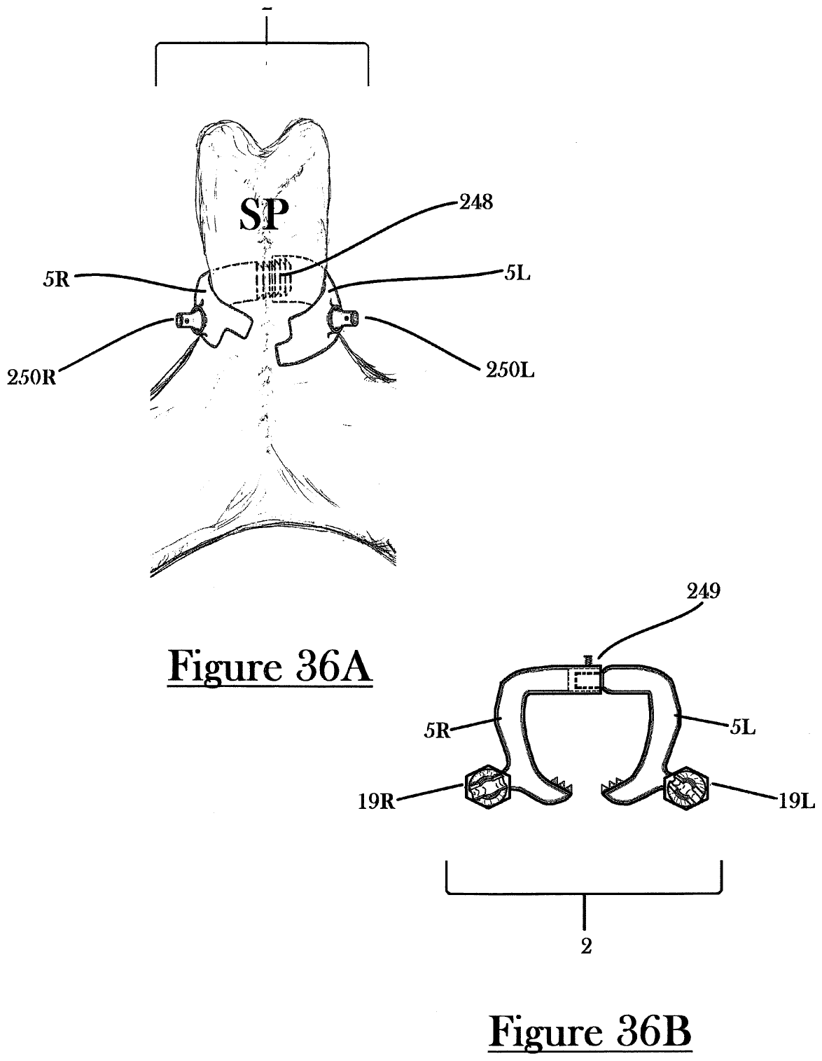

In another embodiment, shown in FIGS. 36A/B, embodiments of the spinous anchor 2 in which the members 5R, L are coupled along the caudal aspect of the Spinous Process (labeled SP in FIG. 36A). In the first image, there is a ratcheting mechanism 248 which couples members 5R and 5L, while unique “ball and socket,” embodiments 250R, L have been provided to the members to couple with the elevating stabilizers. In FIG. 36B, a stabilizing locking screw mechanism 249 has been provided to couple members 5R, L. The preferred embodiment of the mechanism 19R, L to couple with the elevating stabilizers has been provided to the members.

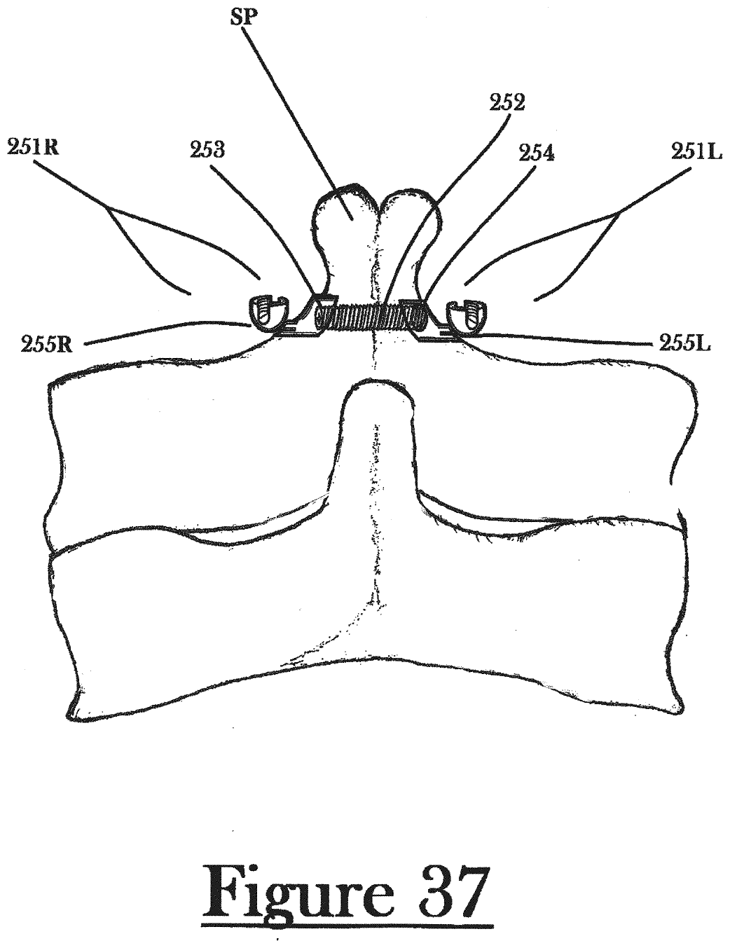

The members securing to the Spinous Process SP do not have to couple to form a single anchor, but rather can be be independent, as shown in several embodiments above. In FIG. 37, these independent anchoring members 251R, L are secured to the SP, and to each other by screw 252. This is introduced through an aperture 253 which in this depiction is positioned in the right member 251R, is disposed through the SP and is secured within a receiving dock 254 provided to the left member 251L. Of course, this could be configured such that the screw would pass in the opposite direction. Mechanisms 255R, L to couple with the elevating stabilizers are also provided to the anchoring members 251R, L.

It is noted that any combination of the aforementioned alternative embodiments would also be included within the Spirit and Scope of the invention. Furthermore, those familiar with the art may anticipate, conceive or propose additional embodiments not included herein; all such embodiments would also be incorporated within the Spirit and Scope of this specification.

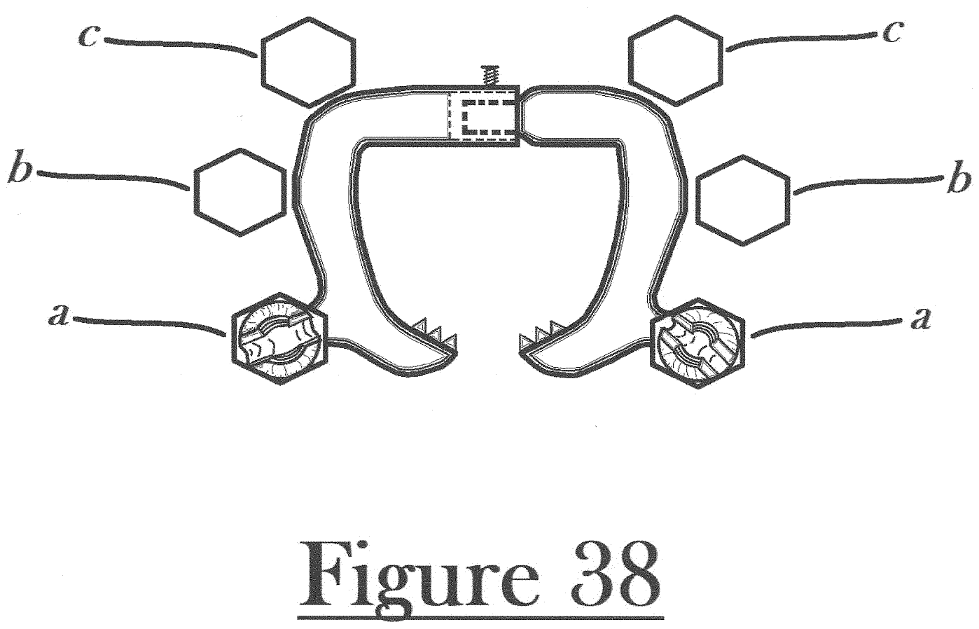

Another aspect of the CMIL for which multiple alternative embodiments can be anticipated is the coupling of the elevating stabilizers with the anchors, beginning with different positions of the coupling of the anchor with the elevating stabilizers. Alternative positions for this mechanism are seen in FIG. 38, where it can be seen that this can be located at the cranial end a of the anchor 2, the midportion b, or at the caudal end c.

A particular challenge in terms of the elevating stabilizers recognizes the mechanics of maintaining the coupling of the construct while the spinolaminar arch is elevated. A brief review of these mechanism shows that the angles of the stabilizing elevators would change with the change in the anteroposterior position of the spinolaminar arch. Furthermore, not only would the angles change but the length of the shaft of the elevator may also change and would have to be accommodated.

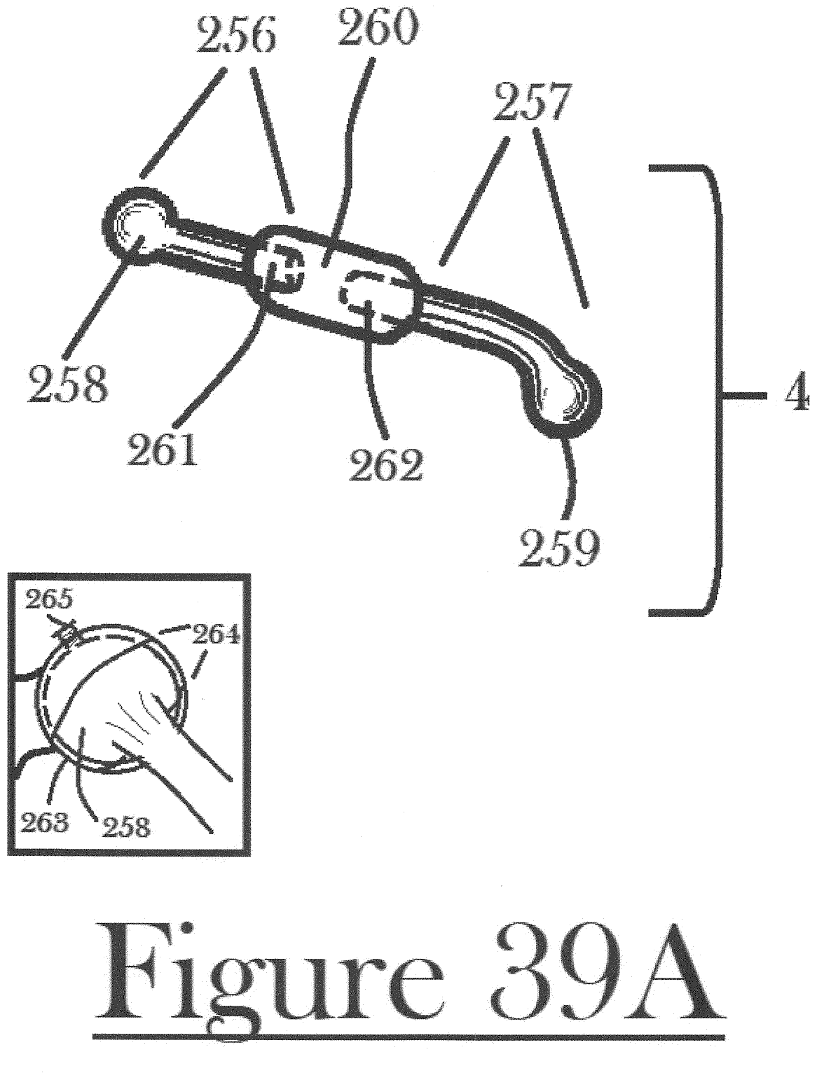

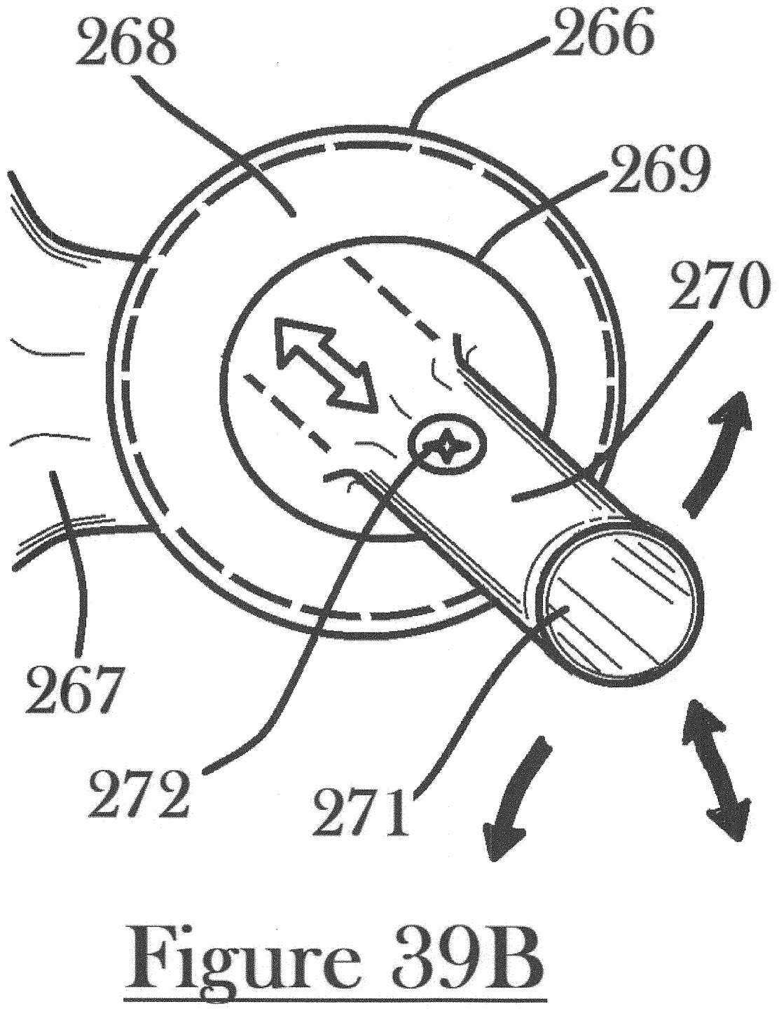

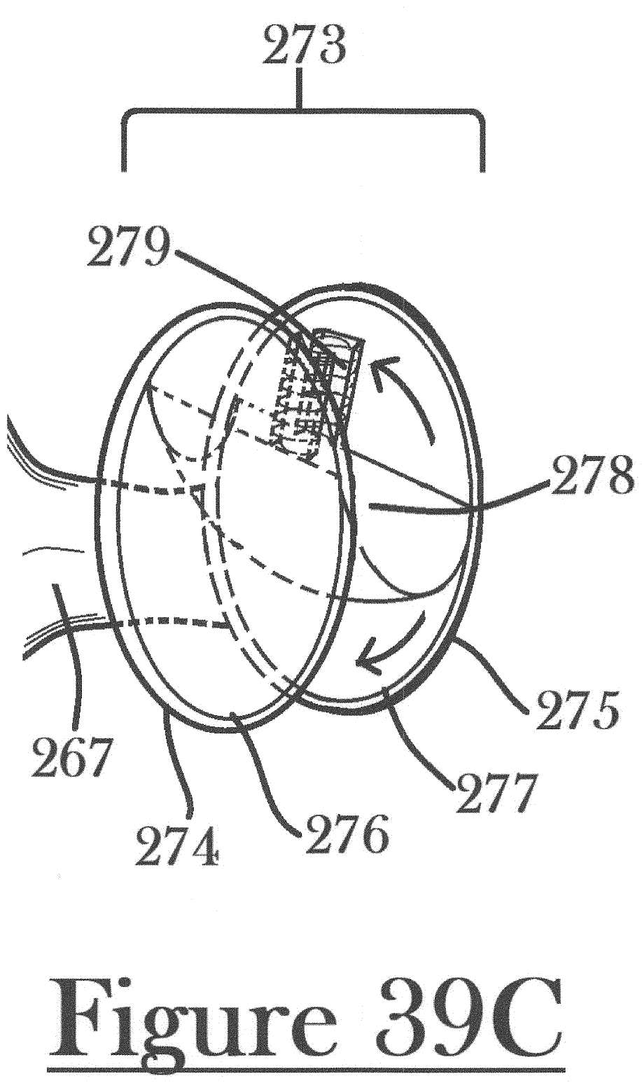

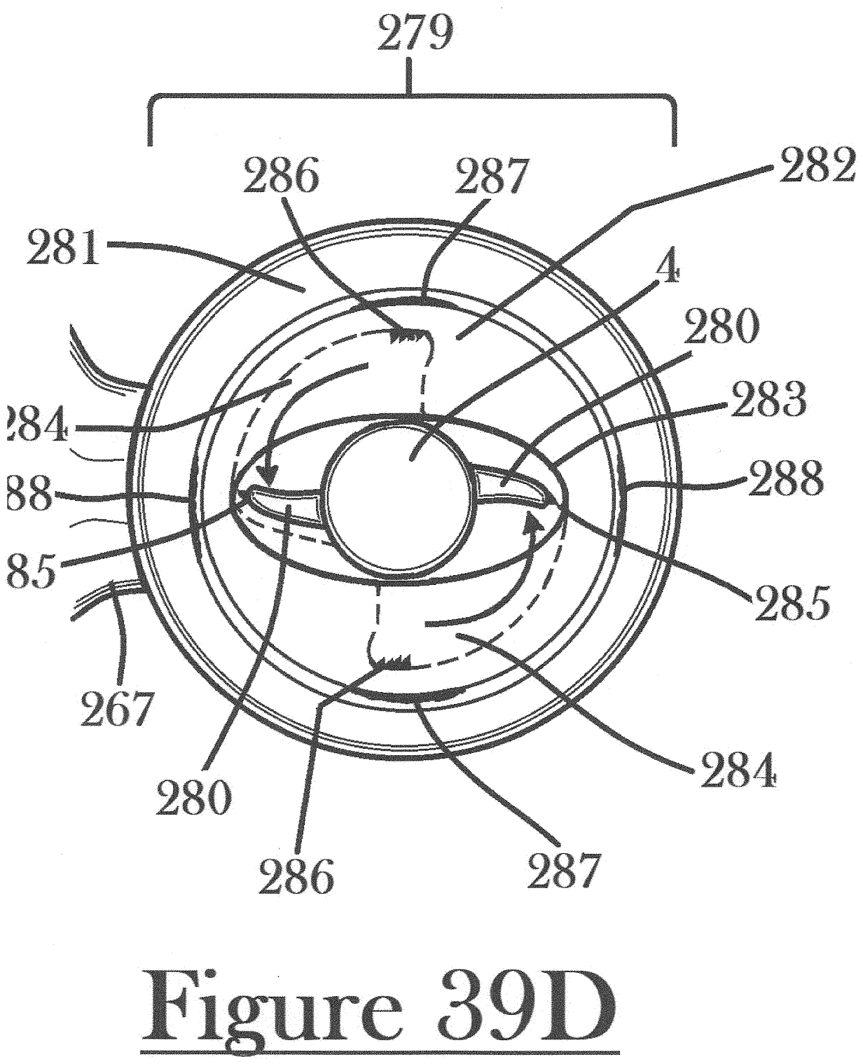

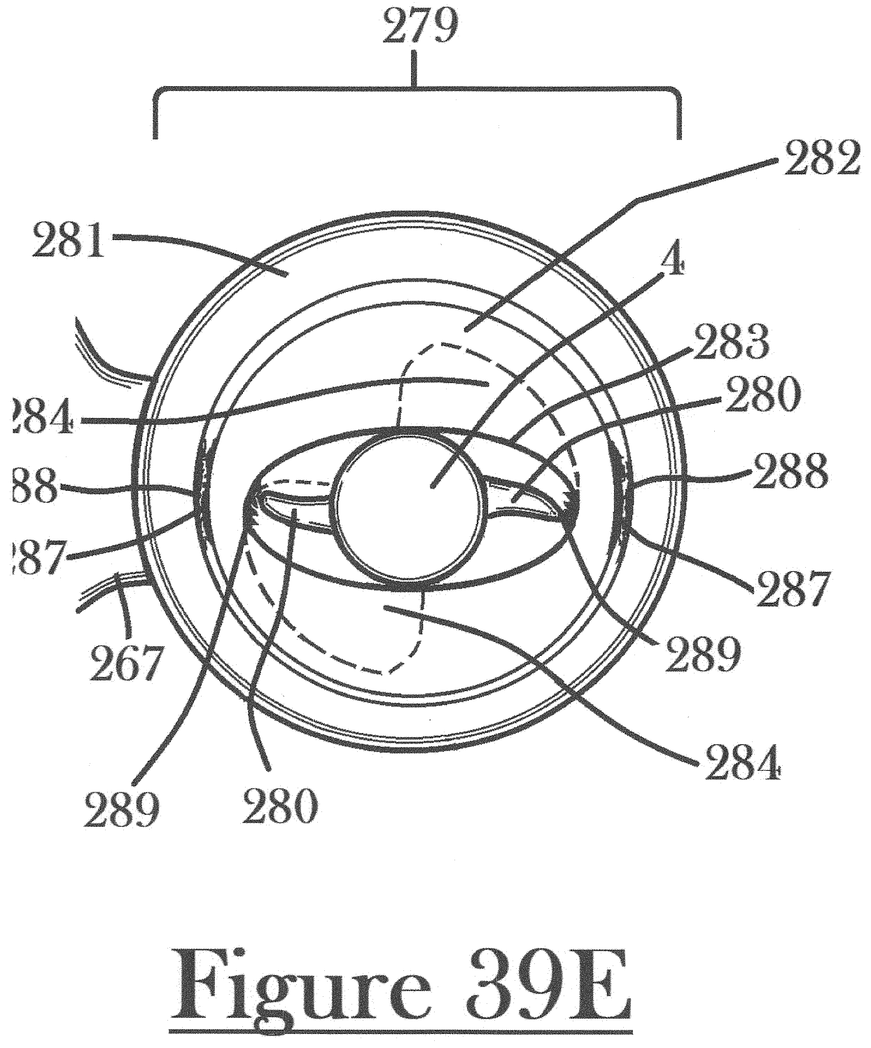

To that end, the preferred embodiment utilizes spherical receiving cradles on both the spinal and lateral anchors which in turn couple with the shaft-like ends of the elevating stabilizer. An alternative, which actually may be preferable in some settings, is to provide the elevating stabilizer with a spherical embodiment on both ends, as seen in FIG. 39A. These would be positioned within rounded tulips or cradles provided to the anchors (insert). The stabilizer 4 demonstrated herein is comprised of a spinous component 256 and a lateral component 257, which are each comprised of a spherical trailing end 258, 259 and shaft-like leading ends 261, 262 which fit into a cold-weld coupler 260. As the spinolaminar arch is elevated, the change of the angles of the stabilizer-anchor coupling is accommodated by the spherical ends 256, 257, while the potential change in the length of the stabilizer 4 is accommodated by cold-weld design, which allows the leading ends 261, 262 of the stabilizer components 256, 257 to be repositioned as necessary. Once the final position is reached, the cold-weld coupler 260 is rotated, achieving the cold-weld and locking the construct.

The insert in FIG. 39A reviews the configuration of the spherical ends within spherical cradles. In this insert, the spinous spherical end 258 is exemplified and is accommodated within a spherical cradle 263, the inner diameter of which is just slightly larger than the outer diameter of sphere 258. Furthermore, a window 264 is provided to the cradle, this window being large enough to permit the stabilizer to be repositioned as anticipated; however, this window is sufficiently small to constrain the sphere 258 within the cradle 263. Once the final position has been achieved, locking screw 265 retains the position of the sphere 258 within the cradle 263.

Other alternative embodiments of the stabilizer can be anticipated; several additional such embodiments are illustrated in FIGS. 39B-E. In one example, shown in an elevational perspective in FIG. 39B, a construct similar to the embodiment described in FIG. 39A, wherein a spherical cradle 266 is provided to the end of a stem-like extension 267 which arises from the anchor. The cradle 266 encases a sphere 268 (depicted by the interrupted circle) which is rotatable within the cradle 266, as indicated by the solid arrows, that being the mechanism accommodating the change in angle inherent to the elevation of the spinolaminar arch. The sphere is provided with a tubular extension 270, provided with a channel 271 configured to accommodate the elevating stabilizer. The relationship between the extension 270 and a window 269 in the cradle 266 creates the range of motion of the sphere 268. The channel 271 of the tubular extension 270 continues through the rotatable sphere 268, indicated by the interrupted straight lines. This accommodates the change in length of the elevating stabilizers seen with elevation of the spinolaminar arch, as indicated by the bidirectional open arrow. Once the final position has been achieved, the elevating stabilizer is secured in place by the securing screw 272.

It is likely that when the spinolaminar arch is elevated, the only angle that actually changes is the slope of the elevating stabilizer as it relates to the cradle; rotational and other angles are unlikely to occur. Therefore, it is arguable that rather than a spherical cradle, a disc-shaped cradle to accommodate the elevating stabilizer may be more efficient. Such a cradle is portrayed in the elevational perspective depicted in FIG. 39C, where it can be seen that again a stem-like extension 267 from the anchor supports the disc-shaped cradle mechanism 273. This is comprised of anterior 274 and posterior 275 non-mobile rims which are repositionably coupled with anterior 276 and posterior 277 rotating discs; these are in turn irreversibly and immovably coupled with each other by a central rod accommodating cradle 278, which is obviously configured to accommodate the elevating stabilizer therethrough. As the angle of the elevating stabilizer changes in association with repositioning the spinolaminar arch, the rotating disc-rod cradle complex can repositionably accommodate this change—as indicated by the curved arrows. Once the final positions are determined, the elevating stabilizer is locked into position with a securing screw (not shown) which is disposed through a tract 279 which is provided to the mechanism.

Another alternative embodiment seen in FIG. 39D combines features provided to both the leading end of the elevating stabilizer 4 and a cradle mechanism 279. As has been previously described, the mechanism 279 is attached to the anchor by a stem 267, as seen in this direct frontal perspective providing a cross-sectional image of the components; herein it can be seen that an outermost, immobile spherical shell 281 is, in the preferred embodiment, continuous and monolithic with the stem 267 arising from the anchor. Closely relating to the outer shell 281 is an inner sphere 282 which is freely rotatable, thereby accommodating any adjustment in the angle of the stabilizer 4. The inner sphere 282 is configured to accommodate the leading end of the stabilizer 4, which is provided with a plurality of locking fins 280 that arise orthogonally from the stabilizer 4 and ideally, are positioned diametrically opposed from each other, as shown. This configuration dictates the need for the ovoid aperture 283 provided to the inner sphere 282, which is continuous with an irregular shaped chamber 284, that being configured to engage with the locking fins 280. This is accomplished by providing the free ends of the fins 280 with ratchet prongs 285 which are designed to engage ratcheting mechanisms 286 provided to the inner surface of the chamber 284. After the stabilizer 4 has been brought into its final position in association with repositioning the spinolaminar arch, the inner sphere is rotated, as indicated by the curved arrows in FIG. 39D. This rotation will not only lock the fins 280 with the ratchet mechanism of the chamber 286, but also actuate a cold weld mechanism provided which has been provided to the interface between the outer surface 287 of the inner mobile sphere 282 and the inner surface 288 of the immobile, spherical shell 281. Rotation of the inner, mobile sphere 282 can be accomplished by any number of fashions, the result of which is seen in FIG. 39E, where it can be seen that the rotation of the mobile sphere 282 has effected rotation of the chamber 284 locking the fin 280/chamber 284 mechanism 289, as well as actuating the cold weld surfaces 287, 288 causing the mobile sphere 282 to be locked within the immobile shell 281. Multiple other mechanisms to lock the elevating stabilizer in place can be anticipated, particularly by those familiar with the art; all such embodiments are incorporated within the spirit and scope of this invention.

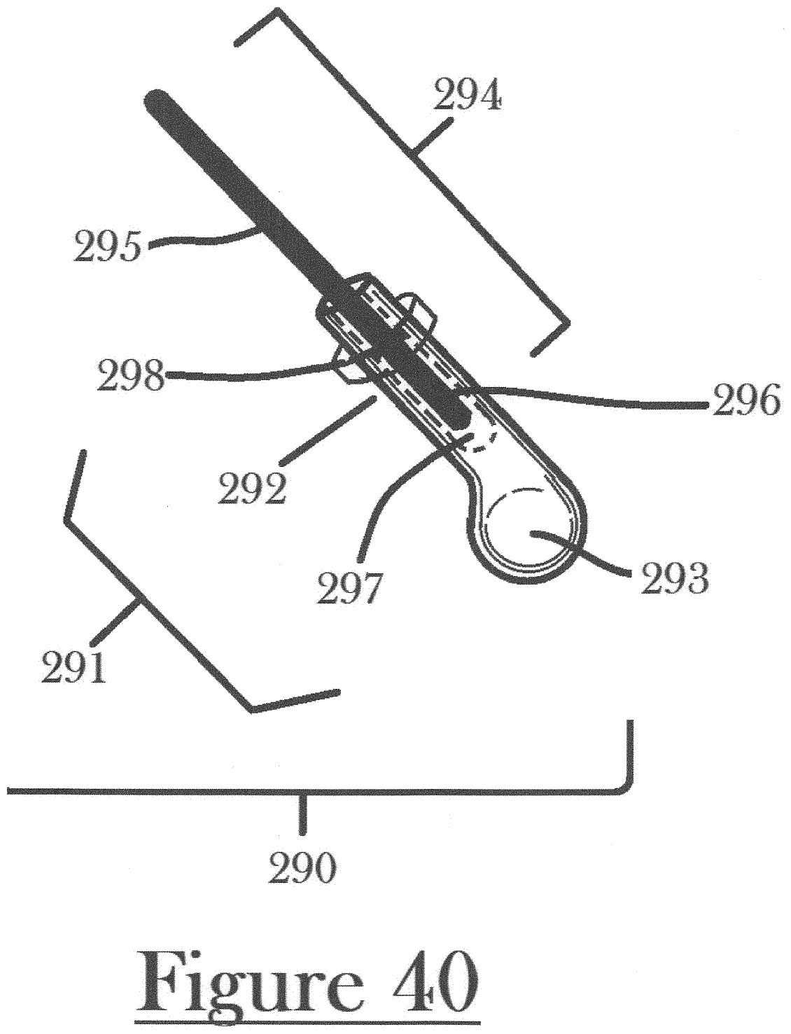

A further variation is shown in the elevated view in FIG. 40, wherein one notes the stabilizer 290 is comprised of two elements, a mobile element 294 and a base element 291, each of which, in turn, are comprised of leading 292, 295 and trailing 293, 296 ends. The trailing end 293 of the base element 291 is provided with a spherical configuration that couples with an anchor; the leading end 292 thereof is provided with a chamber 297 which accommodates the trailing end 296 of the mobile element 294. The [free] leading end 295 of the mobile element 294 is configured to couple with one or more of the anchor mechanisms disclosed in this specification. The mobile element 294 can be telescopically repositionable therein through the chamber 297 such that once the final configuration of the construct has been achieved, the mobile 294 and base 291 elements are locked together by a cold weld mechanism 298, or any other locking mechanism known or acceptable to the art.

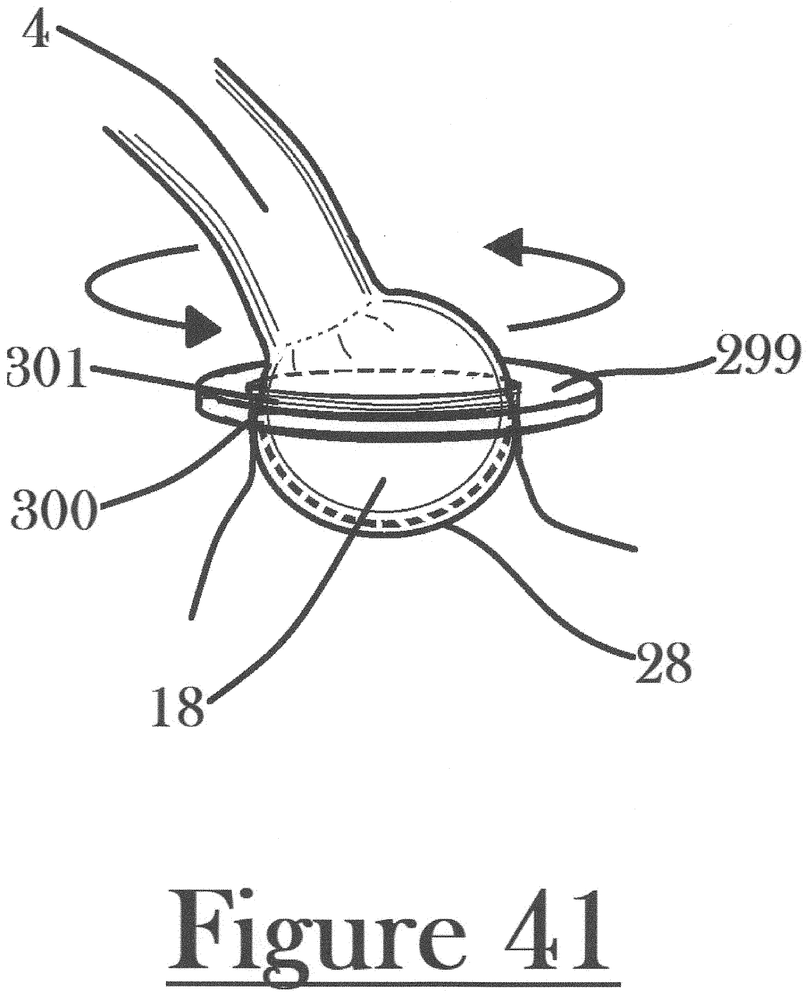

In another aspect of the invention, alternative embodiments of the coupling of the trailing end of the elevating stabilizer with the lateral anchor can be anticipated. One such embodiment is portrayed in FIG. 41, a frontal elevational view showing the spherical trailing end 18 of the stabilizer 4 resting within the socket 28 provided to the anchor. In the preferred embodiment previously disclosed, a securing screw is utilized to stabilize the construct; in this embodiment, threading is provided to the free end 300 of the socket 28. Furthermore, a tightening ring 299 is positioned once the spherical embodiment 18 is within the socket 28. After the final configuration of the construct is assumed, the tightening ring, which has also been provided with threading (not shown), is actuated (arrows) securing the sphere 18 against the inside of the socket 28.

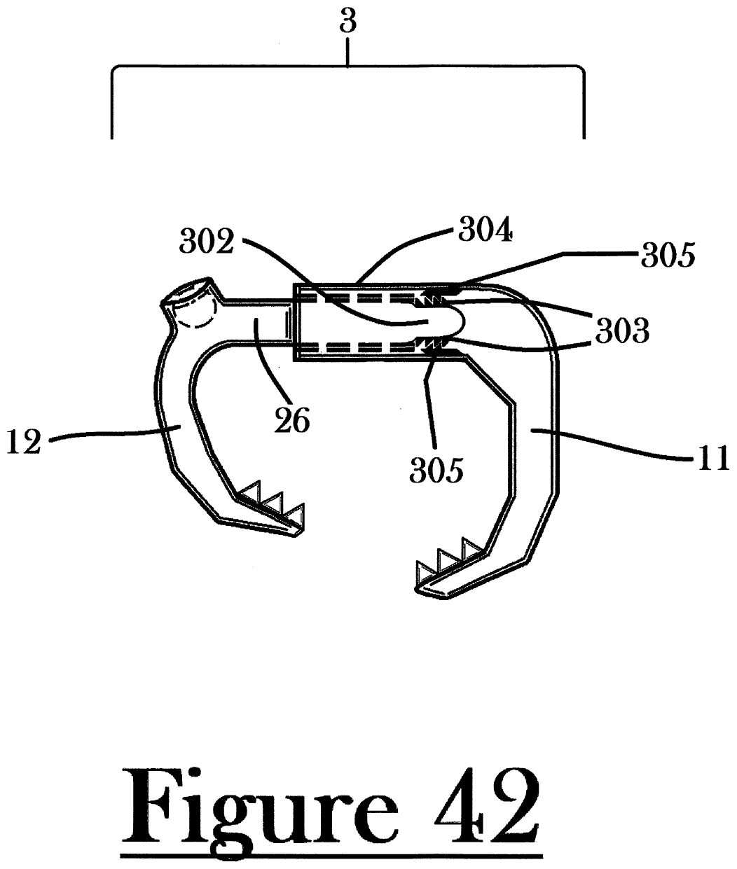

In one such embodiment, depicted in a frontal view in FIG. 42, the anchor is similar to the preferred embodiment insomuch that it is comprised of a lateral 11 and a medial 12 element, but differs in the manner by which these couple and are secured together. In this embodiment, the medial element 12 is provided with a horizontal segment 26 that is disposed into the chamber at the leading end 304 of the lateral element 11. The leading end 302 of the medial element 12 is provided a ratcheting mechanism 303 which is designed to interface with a ratcheting mechanism 305 provided to the leading end 304 of the lateral element 11. These ratcheting mechanisms are actuated as the two elements are compressed together, thus maintaining the lateral anchor in position.

FIG. 43 reveals a frontal perspective of yet another embodiment in which the lateral surface the horizontal segment 306 of the medial element 12 is provided with a cold weld surface 310 and which is disposed into a chamber 308 within the horizontal segment 307 of the lateral element 11. Once the anchor 3 is securely brought against the target bony surface, a rotatable cold weld locking mechanism 310 is rotated to appose the cold weld surfaces 310, 311 against each other, as indicated by the arrows, locking the configuration in position. Other embodiments such as a spring-loaded locking pin, a worm-gear modulated mechanism, and others, all of which are incorporated within the spirit and scope of this invention.

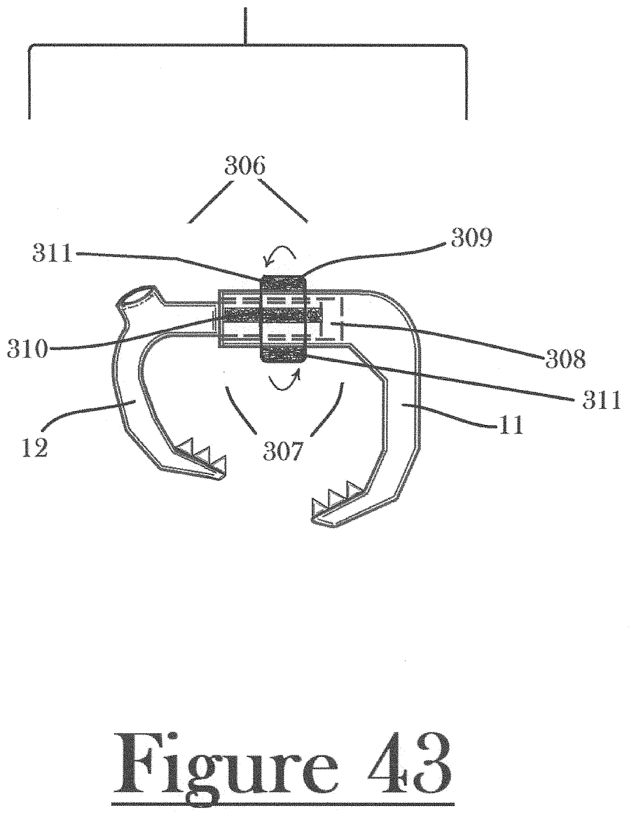

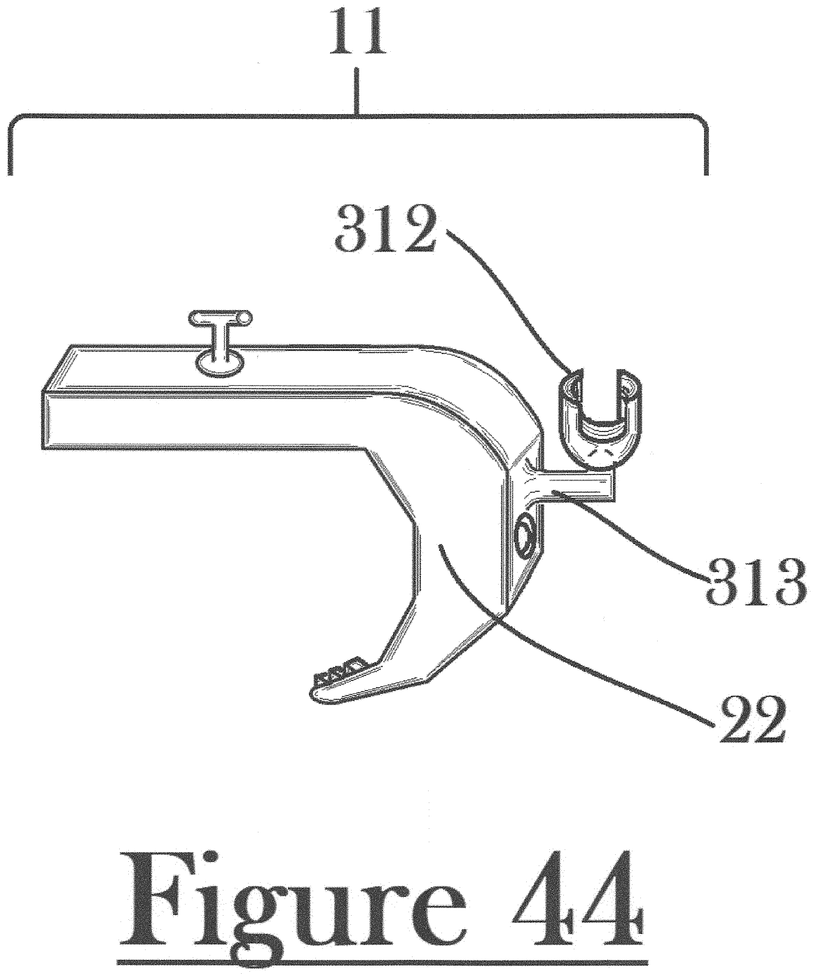

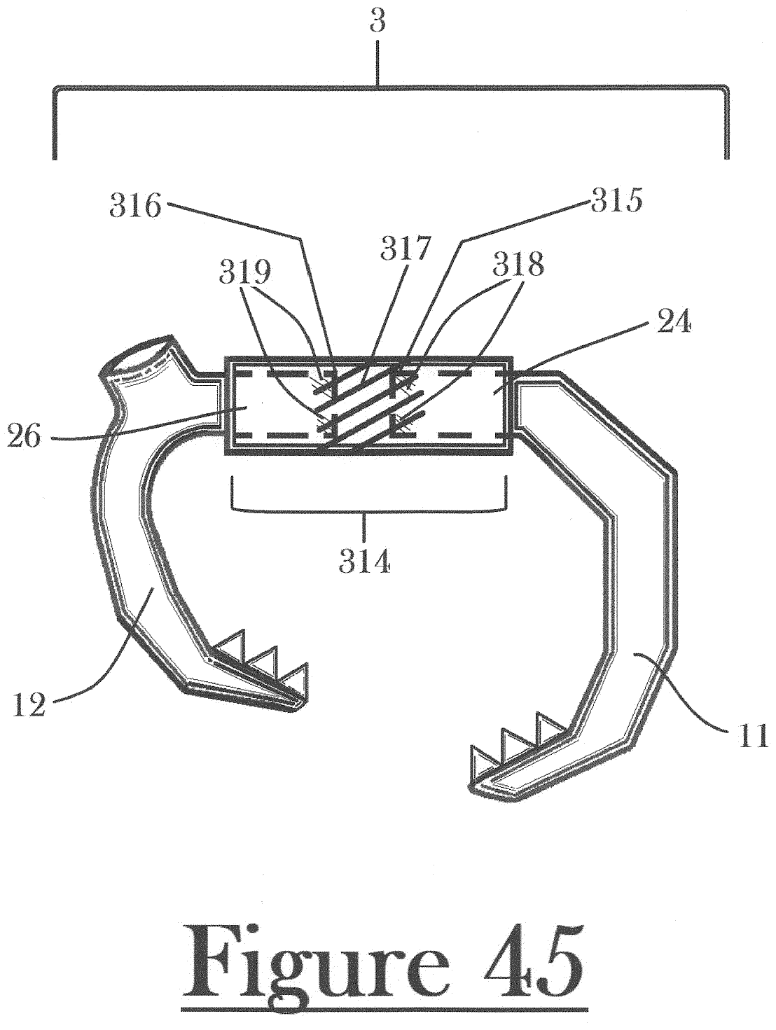

Another element of the lateral anchor which can be anticipated to assume multiple alternative is the position of the cradle which accepts the rod element connecting multiple levels. One alternative embodiment of the lateral element 11 is seen in FIG. 44, a frontal elevational view showing that the cradle 312 has been moved from the horizontal segment such that the supporting arm 313 is seen extending from the vertical segment 22, with the cradle 312 now positioned in the lateral gutter. This would be considered by some to be advantageous in that it would provide an extremely “low profile,” to the construct.

Still another embodiment which can be anticipated is one in which the lateral 11 and medial 12 elements are completely independent and rather than coupling to each other, the free ends 315, 316 of the horizontal segments 24, 26 are each coupled to a third, independent coupling element 314. After the anchor 3 is securely brought against the target bony areas, the coupling element 314 is rotated such that a cold weld surface 317 which has been provided to the inner surface of the coupling element 314 is brought against cold weld surfaces of the free ends 315, 316. Actuation of this coupling element 314 results in locking the entire anchor 3 in position. Of course, other mechanisms can be conceived by which the anchor can be locked into final position.

An alternative embodiment of the device which achieves the lateral osteotomies is portrayed in FIG. 46. This is a lateral perspective of a device which shall be hereinafter referred to as a laminotome 320, which is comprised of a vertically oriented handle 321 which would be guided by surgeon, and can be optionally provided with finger grips 322, as portrayed herein. Clearly, other configurations of the handle 321 can also be anticipated and are incorporated within the spirit and scope of the invention. Regardless, this handle 321 gives rise to an extension, the drill stabilizing extension 325, which is directed substantially orthogonally from the handle 321. Furthermore, the handle 321 then continues in a vertical orientation until it is curved at the base, whereupon it is continuous and monolithic with an attenuated, curvilinear extension, the dural guard 323, which extends substantially parallel to the stabilizing extension 325. Orthogonal to these extensions and parallel to the handle 321 is a vertically oriented monolithic element, the laminar drill 326. This is comprised of a trailing end 327 which serves as the handle by which the surgeon rotates the element 326 (as indicated by the curved arrows), thus actuating the drill 326. The handle 327 then is continuous with a shaft 328, which is disposed through a channel 331 in the drill stabilizing extension 325. The channel 331 then extends through a cone-shaped vertical downward process of the drill stabilizing extension 325, this process being known as the drill introducer 332, which further stabilizes the leading end 329, a side-cutting drill, as it achieves the desired osteotomy. A drill base 330 is provided to the dorsal surface of the dural guard 323 which is configured to receive the side-cutting drill 329 so that the free end of the drill is not freely spinning.

The dural guard 323 is designed such that its leading edge 324, which comes to a tapered point, caoduced under (anterior to) the lamina which is to be osteotomized and can be insinuated into the plane between the lamina and the yellow ligament. The curvilinear configuration then deflects the yellow ligament and dura away the laminotome 320 as the osteotomy advances, thus preventing any inadvertent dural lacerations.

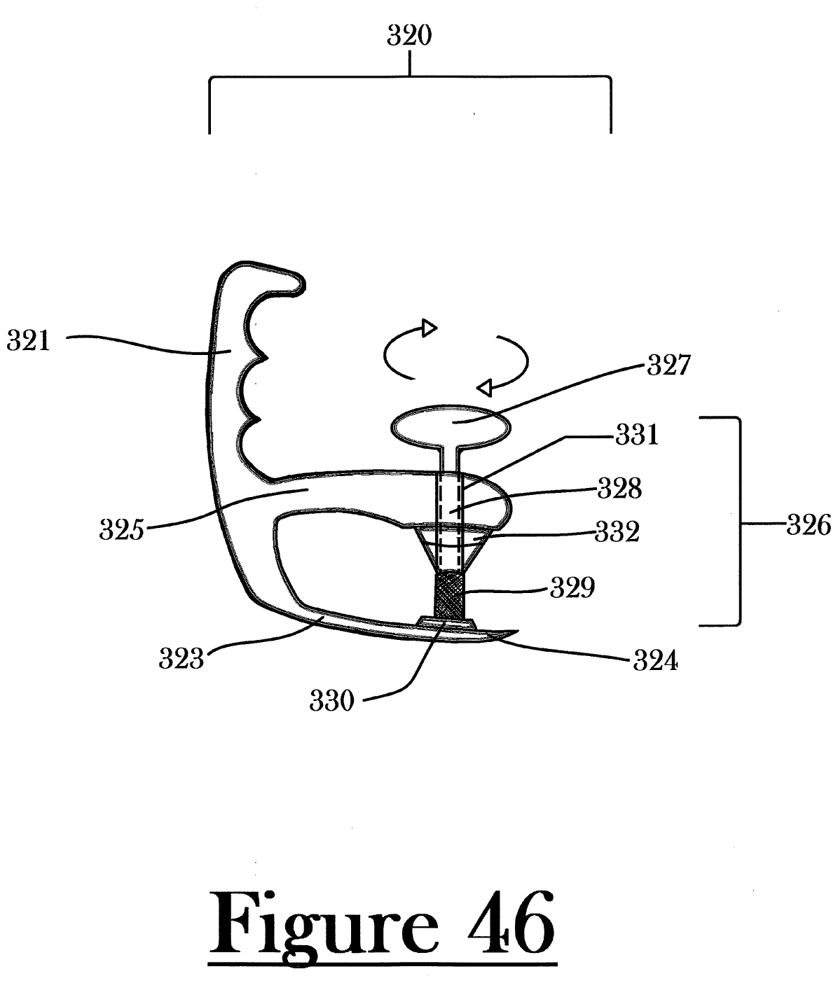

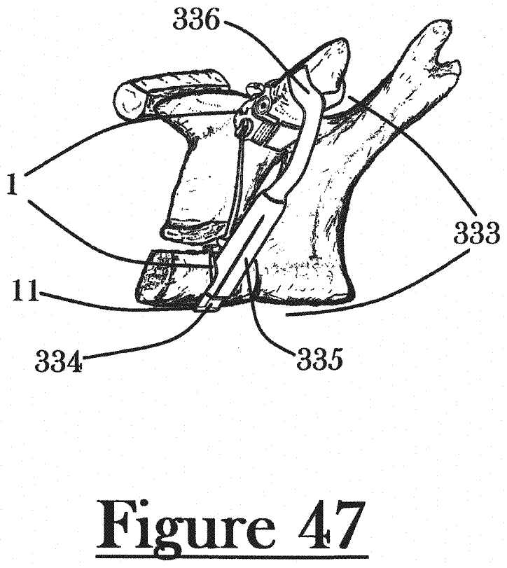

As illustrated in FIG. 47, a lateral elevational view of a spinal segment in which a CMIL 1 has been secured to the more cranial vertebra, but the spinolaminar arch has not yet been elevated into the final position. A device known as the Lateral Arch Elevator 333 is reversibly coupled to the lateral element 11 of the lateral anchor, as well as the tip of the spinous, and is comprised of two elements, the stabilizing base 334 which reversibly couples to the lateral element 11, and the elevating element 335, which is slidably, repositionably coupled to the stabilizing base 334. The elevating element 335 is monolithic and continuous with an arm, the spinous anchor coupler 336, which reversibly couples with the spinous process such that actuation of elevating element 335 results in elevation of the spinolaminar arch, achieving the objective of the surgery.

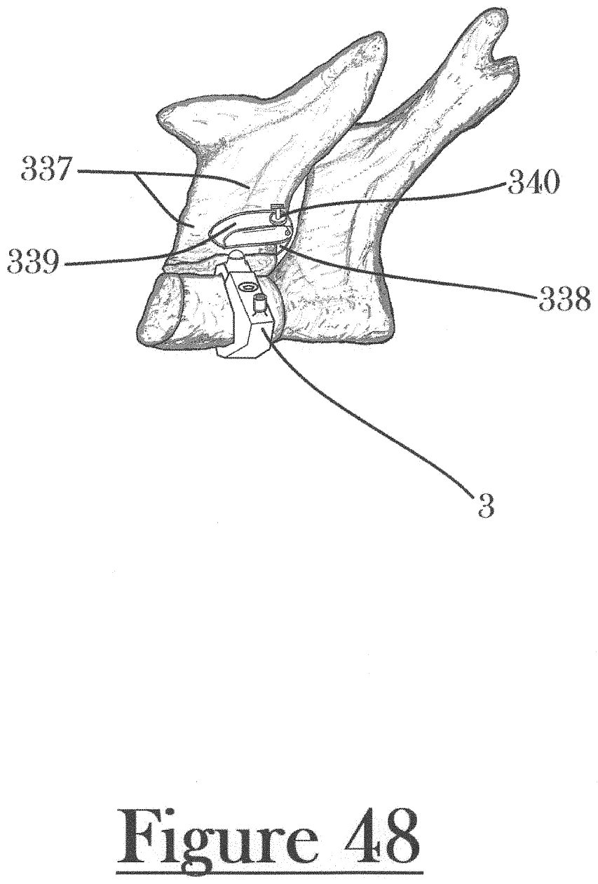

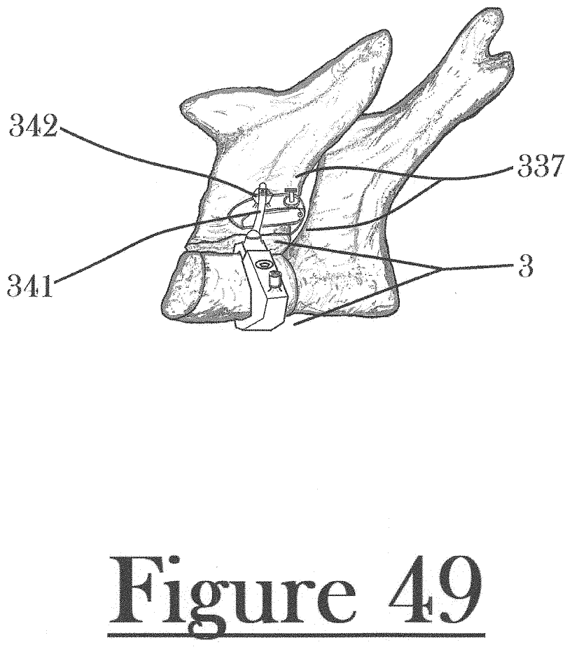

In FIG. 48, an alternate embodiment of the system is demonstrated in the lateral elevational view wherein a lateral anchor 3 as seen in the preferred embodiment is again in position, but rather than a spinous anchor, one notes a laminar anchor 337 which is comprised of a pair of jaws which are passed around the caudal edge of the lamina. In furtherance of this description, a sublaminar jaw 338, of which only the caudalmost aspect is seen, is passed under the lamina while the dorsal laminar jaw 339 is brought against the dorsal surface of the lamina. A securing screw 340 is then actuated, compelling the two jaws 338, 339 towards each other and achieving excellent fixation against the lamina. In FIG. 49, a rod coupling mechanism 342 has been provided to the laminar anchor 337, and a connecting rod 341 has coupled the two anchors together after elevating the spinolaminar arch. This embodiment has the advantage of preserving the central portion of the spinolaminar arch; hence, the construct can be implanted through two limited lateral incisions.

A system of devices to provide novel surgical methods for use for establishing a decompressive laminoplasty at one or more levels of the cervicothoracic spine comprising a first device for achieving precisely placed bilateral laminotomies; laminar anchors which can be placed on the lateralmost aspects of the laminae which have undergone laminotomy bilaterally, and in that way, creating a spinolaminar arch; anchors which can be placed on the lateral masses which have been divided from the laminae bilaterally; elements which couple each pair of laminar anchors and lateral mass anchors in a rotatable, slidable manner; and a system for changing the positions of the laminar anchors with respect to the lateral mass anchors resulting in elevation of the spinolaminar arch created by the laminotomies, this elevation resulting in decompression of the neural elements contained within the spinal canal; a device for locking the anchors in position with respect to one another once the desired decompression has been achieved.

In particular the system includes means for precisely placed laminotomies using a substantially flattened, plate-like component, which is configured to be placed against the posterior surface of a target lateral mass, which can be monolithic and continuous with a curved portion which is configured to be brought against the lateral aspect of the lateral mass. It may have calibrations visible on the dorsal surface of the device.

As part of the system there is included a tube-like cylindrical drill guide with a leading end and a trailing end, the leading end being slidably couple able the plate-like component of the Device to achieve precisely positioned laminotomies by one or more arc-like couplings The tube like guide can be cylindrical drill guide can be repositioned in the mediolateral axis so that the leading end of the tubular drill guide can be positioned in accordance with coordinates dictated by the system for evaluating preoperative data. The plate-like component of the Device for achieving precisely positioned laminotomies in claim 2, which serves as a mediolateral guide for positioning the tube-like cylindrical drill guide.

In certain forms the system includes an arc system, which couples the plate-like component of the device for achieving precisely positioned laminotomies with the tube-like cylindrical drill guide to confer angular adjustability on the tube-like cylindrical drill guide such that the precisely planned angle as dictated by the system for evaluating preoperative data so that the desired position of the laminotomies is achieved.

The system also includes a rotatable drill configured to be disposed through the tubular drill guide. The guide can be adjusted to the prescribed mediolateral and angular positions. This guided drill is used to creates a laminotomy which extends from the inferior edge of a target lamina to the superior end of the lamina at the junction of the lateral edge of the lamina with the medial edge of the lateral mass.

The rotatable drill is substantially elongated and configured to be disposed through the tubular drill, and is monolithic and provided with a leading end, a shaft extending from the leading end to a trailing end. The leading end has a circumferentially roughened surface which is of sufficient configuration to achieve an osteotomy of approximately the width the drill. The drill shaft has a leading end and the trailing end where the trailing has a rotatable handle, by which the surgeon can actuate the leading end of the rotatable drill and in that fashion.

The system also include a laminar anchor made of a sublaminar jaw, a dorsal laminar jaw, coupled by a transverse axis, and a screw which is positioned to actuate the anchor such that rotation of the screw compels the jaws to approach each other and form a grip around said target lamina and in that way create a secure clamp against the inferior edge of the target lamina.

More specifically, the sublaminar jaw is a thin plate with a leading end, a central body, and a trailing end, and is configured to be insinuated along the anterior surface of the target lamina. The leading may be provided with small teeth-like ridges or corrugations, these corrugations configured to create additional friction against the cortical surface of the lamina without violating that cortical surface. The body of the sublaminar jaw is configured to be positioned against a substantial area of the sublaminar cortical surface of the target lamina. The trailing end of the sublaminar jaw is configured to couple with the dorsal laminar jaw.

The trailing end is provided with apertures on its lateral aspects which are configured to accommodate an axle or axis, disposed through these apertures as well as apertures in the dorsal laminar jaw. The trailing end is also provided with a channel which is oriented in the posterior-anterior axis. The axle or axis orthogonally couples the sublaminar jaw with the dorsal laminar jaw. The interior surface of both sublaminar jaw and dorsal laminar jaw are designed to conform to the target areas of the lamina, and they go from narrow at their tips to broader near the joining axis. The jaws can be rotated towards or away from each other through the axle or axis.

The dorsal laminar jaw has a leading end, a central body, and a trailing end. The leading end may have tooth-like projections or ridges or roughened surfaces configured to create additional friction against the bony cortical surface but are specifically configured so as to not penetrate the cortical surface or bone proper. Whereas the central body of the dorsal laminar jaw is configured to be positioned against a substantial area of the dorsal laminar cortical surface of the target lamina. The trailing end of the dorsal laminar jaw is connected to the sublaminar jaw with a screw means or other adjustable mechanism. A threaded channel in the trailing end of the dorsal laminar jaw into the channel in the trailing end of the sublaminar jaw.

The system also includes a dedicated implantation instrument having an outer cannulated element and an inner rotatable element. The outer cannulated element of the implantation instrument can be utilized to direct and stabilize the leading end of the instrument, an elongated central shaft and a leading end which is provided with a mechanism to reversibly couple with the laminar anchor is order to position the anchor against the inferior/caudal edge of the lamina, and release the anchor once the anchor is secured against the lamina. A central rotatable screwdriver is positioned within the cannulated element which has a leading end, that leading configured to reversibly couple with a locking nut which is configured to be secured against the trailing end of the screw. Further adjustable means are provided in the instrument to preventing unintended reverse rotation and backout of the screw.

The dorsal laminar jaw of the laminar anchor, the dorsal surface of which is provided with a housing unit which is configured to accept a sphere-like leading end of a coupling element which couples the laminar anchor with the lateral mass anchor and a means of locking the leading end of the coupling element in place once the final position of the spinolaminar arch has been determined. The coupling element has a spherical leading end, an elongated central shaft and a spherical trailing end, with the leading and trailing ends configured to be housed within housing units provided to the laminar and lateral mass anchors.

The lateral mass anchor has a medial element and a lateral element, those elements being slidably coupled to each other in a fashion so that they may be secured against the lateral mass of a target vertebra. The medial of the lateral mass anchor is configured to be insinuated through the laminotomy positioned between the lateral aspect of the lamina and the medial aspect of the lateral mass, and is substantially a flattened, plate-like structure with a leading end, a central body and a trailing end. It may be substantially “C-shaped,” as seen in the frontal view. The medial element of the lateral mass anchor may be configured with an inclination towards the anteriormost aspect of the medial surface of the lateral mass and may have ridges, corrugations, or tooth-like projections to increase the friction against the bony surface of the lateral mass and is shaped to be brought against the medial surface of the lateral mass.

The trailing end of the medial element is continuous with the central body through an approximate 90-degree bend such that the trailing end is oriented orthogonal to the central body and is configures to slidably couple with the lateral element. It is also substantially “C-shaped,” in configuration, and furthermore, is substantially a “mirror image,” of the medial element. The lateral element is also provided with a leading end which may be provided with a slight incline inwards towards the most anterior aspect of the lateral mass, this leading end which also may be provided with ridges, corrugations, or tooth-like projections that are specifically configured to increase the friction against the cortical surface of the lateral mass.

The lateral element has a central body, which may be somewhat expanded and flattened to be brought against the lateral surface of the lateral mass. The trailing end which is monolithic and continuous with the central body through an approximately 90 degree angulation, such that the trailing end is essentially orthogonal to the central body; furthermore, the trailing end is provided with a configuration through which the lateral element can be slidably coupled with the medial element. Means for locking the lateral and medial elements to each other once an appropriate position of the lateral mass anchor has been achieved are provided.

The system may also include a housing unit configured to house the lateral end of the connecting element to the lateral mass anchor with the laminar anchor. The housing unit creates a cradle for the trailing end of the medial element of the lateral mass anchor. The cradle has calibrations which relate to preoperative data and determine the amount of elevation of the spinolaminar arch to achieve the target decompression.

The system may also include as part of the lateral element of the lateral mass anchor in hay have a cradle to house a coupling rod, said coupling rod being positioned within the cradles of two or more contiguous lateral mass anchors and in that way, this coupling element serves to stabilize one or more target motion segments. An alternative embodiment, in which there is no laminar anchor, this element having been replaced by a central spinous process anchor, this anchor having been provided with a mechanism to ensure secure fixation against the target spinous process.

What is claimed is:

1. An apparatus or system of devices to provide novel surgical method for use for establishing a decompressive laminoplasty at one or more levels of the cervicothoracic spine comprising: at least two nonintrusive laminar anchors which can be placed nonintrusive on the lateralmost aspects of a laminae which have undergone laminotomy bilaterally creating a spinolaminar arch; nonintrusive lateral mass anchors which can be placed on the lateral masses which have been divided from the laminae bilaterally; at least two connecting elements which couple each pair of laminar anchors and said lateral mass anchors in a rotatable, slidable manner; wherein changing the positions of the laminar anchors with respect to the lateral mass anchors resulting in elevation of the spinolaminar arch created by the laminotomies where the elevation resulting in decompression of the neural elements contained within the spinal canal; means for locking said lateral mass laminar anchors and said anchors in position with respect to one another once the desired decompression has been achieved.

2. The system of claim 1 where the device for precisely placed laminotomies comprises a substantially flattened, plate-like component, which is configured to be placed against the posterior surface of a target lateral mass.

3. The device for achieving precisely placed laminotomies in claims 2, which is monolithic and continuous with a curved portion which is configured to be brought against the lateral aspect of the lateral mass.

4. The device for achieving precisely placed laminotomies in claim 3, which is provided with calibrations visible on the dorsal surface of the device.

5. The system of devices of claim 4 further including a device for achieving precisely positioned laminotomies further comprising a tube-like cylindrical drill guide.

6. The tube-like/tubular cylindrical drill guide in claim 5, which is provided with a leading end and a trailing end, the leading end being slidably coupled to the plate-like component of the Device to achieve precisely positioned laminotomies by one or more arc-like couplings.

7. A laminar anchor comprising a sublaminar jaw, a dorsal laminar jaw, wherein those jaws then being coupled by a transverse axis, and a screw which is positioned to actuate the anchor such that rotation of the screw compels the jaws to approach each other and form a grip around said target lamina and in that way create a secure clamp against the inferior edge of the target lamina.

8. The sublaminar jaw in claim 7 of the laminar anchor, which is a thin plate which is itself provided with a leading end, a central body, and a trailing end, and is configured to be insinuated along the anterior surface of the target lamina.

9. The leading end of the sublaminar jaw in claim 8, wherein said leading end is provided with small teeth-like corrugations configured to create additional friction when placed against a chosen target area of the cortical surface of the target lamina without violating that cortical surface.

10. The central body in claim 8, which is configured to be positioned against a substantial area of the sublaminar cortical surface of the target lamina.

11. The trailing end in claim 8 of said sublaminar jaw which is configured to couple with the dorsal laminar jaw.

12. The trailing end in claim 7, which is also provided with a channel which is oriented in the posterior-anterior axis.

13. The axis in claim 7 which is positioned orthogonal to the long axis of the jaws of the laminar anchor.

14. The dorsal laminar jaw in claim 7, which is provided with a leading end, a central body, and a trailing end.

15. The leading end in claim 14 of the dorsal laminar jaw, which is provided with tooth-like projections or ridges which are configured to create additional friction against the bony cortical surface but are specifically configured so as to not penetrate cortical surface of bone proper.

16. The central body of the dorsal laminar jaw in claim 14, which is configured to be positioned against a substantial area of the dorsal laminar cortical surface of the target lamina.

17. The trailing end of the dorsal laminar jaw in claim 14, which is provided with apertures on its lateral aspects which are configured to accommodate an axis disposed through these apertures as well as apertures in the sublaminar jaw that aperture then continuous with a channel which extends through the trailing end of the dorsal laminar jaw and is continuous with the channel provided to the trailing end of the sublaminar jaw.

18. The trailing end in claim 14 of the screw in claim 1 which is expanded and configured to interact with the trailing ends of the sublaminar and dorsal laminar jaws in a manner such that advancing the screw in claim 1 through the apertures compels the jaws towards each other to form a substantially closed clamp around the target lamina.

19. The dorsal laminar jaw in claim 18 of the laminar anchor in claim 1, the dorsal surface of which is provided with a housing unit which is configured to accept a sphere-like leading end of a coupling which couples the laminar anchor with the lateral mass anchor and a means of locking the leading end of the coupling element in place once the final position of a spinolaminar arch has been determined.

20. The lateral mass anchor in claim 1, wherein said lateral mass anchor is provided with a medial element and a lateral element, those elements being slidably coupled to each other in a fashion so that they may be secured against the lateral mass of a target vertebra.

21. The medial element in claim 20 of the lateral mass anchor which is configured to be insinuated through the laminotomy positioned between the lateral aspect of the lamina and the medial aspect of the lateral mass, and is substantially a flattened, plate-like structure with a leading end, a central body and a trailing end.

22. The medial element in claim 21 of the lateral mass anchor which is C-shaped.

23. The leading end of the medial element in claim 22 of the lateral mass which is provided with an inclination towards the anteriormost aspect of the medial surface of the lateral mass.

24. The trailing end in claim 21 of the medial element of the lateral mass anchor which is configured to slidably couple with the lateral element.

25. The lateral element in claim 20 which is also provided with a leading end which may be provided with a slight incline inwards towards the most anterior aspect of the lateral mass, this leading end which also may be provided with ridges, corrugations, or tooth-like projections that are specifically configured to increase the friction against the cortical surface of the lateral mass.

26. The lateral element in claim 20 which is also provided with a central body, which may be somewhat expanded and flattened to be brought against the lateral surface of the lateral mass.

27. The lateral element in claim 20 which is provided with a trailing end which is monolithic and continuous with the central body through an approximately 90 degree angulation, such that the trailing end is essentially orthogonal to the central body; furthermore, the trailing end is provided with a configuration through which the lateral element can be slidably coupled with the medial element.
